SEQUENCE LISTING (58 sequences)

SEQ ID NO: 1 - protein, 5 aa, synthetic construct
GYSMN

SEQ ID NO: 2 - protein, 17 aa, synthetic construct
YISSYESTIYYADSVKG

SEQ ID NO: 3 - protein, 15 aa, synthetic construct
SRITLVRGVPRYFDL

SEQ ID NO: 4 - protein, 11 aa, synthetic construct
RASQSVSSYLA

SEQ ID NO: 5 - protein, 7 aa, synthetic construct
DASNRAT

SEQ ID NO: 6 - protein, 8 aa, synthetic construct
QQRSNWWT

SEQ ID NO: 7 - protein, 8 aa, synthetic construct
GFTFIGYS

SEQ ID NO: 8 - protein, 8 aa, synthetic construct
ISSYESTI

SEQ ID NO: 9 - protein, 17 aa, synthetic construct
ARSRITLVRGVPRYFDL

SEQ ID NO: 10 - protein, 6 aa, synthetic construct
QSVSSY

SEQ ID NO: 11 - skipped

SEQ ID NO: 12 - protein, 8 aa, synthetic construct
QQRSNWWT

SEQ ID NO: 13 - protein, 124 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSS

SEQ ID NO: 14 - protein, 106 aa, synthetic construct
EIVLTQSPGTLSLSPGERATLSCRASQSVSSYLAWYQQKPGQAPRLLIYDASNRATGIPARFSGSGSGTDFTLTISSLQPEDFAVYYCQQRSNWWTFGQGTKVEIK

SEQ ID NO: 15 - protein, 453 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 16 - protein, 213 aa, synthetic construct
EIVLTQSPGTLSLSPGERATLSCRASQSVSSYLAWYQQKPGQAPRLLIYDASNRATGIPARFSGSGSGTDFTLTISSLQPEDFAVYYCQQRSNWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 5 aa, synthetic construct
SRGMS

SEQ ID NO: 18 - protein, 17 aa, synthetic construct
FISSSGTYVYYADSVKG

SEQ ID NO: 19 - protein, 13 aa, synthetic construct
DLPRTGVYFGMDV

SEQ ID NO: 20 - protein, 11 aa, synthetic construct
RASQSIFNYLN

SEQ ID NO: 21 - protein, 7 aa, synthetic construct
GASTLQS

SEQ ID NO: 22 - protein, 9 aa, synthetic construct
QESYSTPYT

SEQ ID NO: 23 - protein, 8 aa, synthetic construct
GFTFSSRG

SEQ ID NO: 24 - protein, 8 aa, synthetic construct
ISSSGTYV

SEQ ID NO: 25 - protein, 15 aa, synthetic construct
ARDLPRTGVYFGMDV

SEQ ID NO: 26 - protein, 6 aa, synthetic construct
QSIFNY

SEQ ID NO: 27 - skipped

SEQ ID NO: 28 - protein, 9 aa, synthetic construct
QESYSTPYT

SEQ ID NO: 29 - protein, 122 aa, synthetic construct
EVQLLESGGGLVQPGGSLRLSCAASGFTFSSRGMSWVRQAPGKGLEWVSFISSSGTYVYYADSVKGRFTISRDNSKNTLYLQMNSLRAEDTAVYYCARDLPRTGVYFGMDVWGQGTTVTVSS

SEQ ID NO: 30 - protein, 107 aa, synthetic construct
DIQMTQSPSSLSASVGDRVTITCRASQSIFNYLNWYQQKPGKAPKLLIYGASTLQSGVPSRFSGSGSGTDFTLTISSLQPEDFATYYCQESYSTPYTFGQGTKVEIK

SEQ ID NO: 31 - protein, 122 aa, synthetic construct
EVQLLESGGGLVQPGGSLRLSCAASGFTFSSRGMSWVRQAPGKCLEWVSFISSSGTYVYYADSVKGRFTISRDNSKNTLYLQMNSLRAEDTAVYYCARDLPRTGVYFGMDVWGQGTTVTVSS

SEQ ID NO: 32 - protein, 107 aa, synthetic construct
DIQMTQSPSSLSASVGDRVTITCRASQSIFNYLNWYQQKPGKAPKLLIYGASTLQSGVPSRFSGSGSGTDFTLTISSLQPEDFATYYCQESYSTPYTFGCGTKVEIK

SEQ ID NO: 33 - protein, 249 aa, synthetic construct
EVQLLESGGGLVQPGGSLRLSCAASGFTFSSRGMSWVRQAPGKCLEWVSFISSSGTYVYYADSVKGRFTISRDNSKNTLYLQMNSLRAEDTAVYYCARDLPRTGVYFGMDVWGQGTTVTVSSGGGGSGGGGSGGGGSGGGGSDIQMTQSPSSLSASVGDRVTITCRASQSIFNYLNWYQQKPGKAPKLLIYGASTLQSGVPSRFSGSGSGTDFTLTISSLQPEDFATYYCQESYSTPYTFGCGTKVEIK

SEQ ID NO: 34 - protein, 720 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGSGGGGSGGGGSGGGGSEVQLLESGGGLVQPGGSLRLSCAASGFTFSSRGMSWVRQAPGKCLEWVSFISSSGTYVYYADSVKGRFTISRDNSKNTLYLQMNSLRAEDTAVYYCARDLPRTGVYFGMDVWGQGTTVTVSSGGGGSGGGGSGGGGSGGGGSDIQMTQSPSSLSASVGDRVTITCRASQSIFNYLNWYQQKPGKAPKLLIYGASTLQSGVPSRFSGSGSGTDFTLTISSLQPEDFATYYCQESYSTPYTFGCGTKVEIK

SEQ ID NO: 35 - protein, 18 aa, synthetic construct
GGSGGGGSGGGGSGGGGS

SEQ ID NO: 36 - protein, 257 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 37 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 38 - protein, 671 aa, synthetic construct
SGGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 39 - protein, 226 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 679 aa, synthetic construct
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 443 aa, synthetic construct
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 42 - protein, 219 aa, synthetic construct
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 452 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSP

SEQ ID NO: 44 - protein, 18 aa, synthetic construct
EIVLTQSPGTLSLSPGER

SEQ ID NO: 45 - protein, 18 aa, synthetic construct
  MOD_RES 1: note = pyroglutamate
XIVLTQSPGTLSLSPGER

SEQ ID NO: 46 - protein, 19 aa, synthetic construct
EVQLVESGGGLVQPGGSLR

SEQ ID NO: 47 - protein, 19 aa, synthetic construct
  MOD_RES 1: note = pyroglutamate
XVQLVESGGGLVQPGGSLR

SEQ ID NO: 48 - protein, 7 aa, synthetic construct
SLSLSPG

SEQ ID NO: 49 - protein, 6 aa, synthetic construct
SLSLSP

SEQ ID NO: 50 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 51 - protein, 231 aa, synthetic construct
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 52 - protein, 231 aa, synthetic construct
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 53 - protein, 222 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 54 - protein, 20 aa, synthetic construct
GGGGSGGGGSGGGGSGGGGS

SEQ ID NO: 55 - protein, 230 aa, synthetic construct
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSP

SEQ ID NO: 56 - protein, 230 aa, synthetic construct
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSP

SEQ ID NO: 57 - protein, 107 aa, synthetic construct [sequence omitted]

SEQ ID NO: 58 - protein, 719 aa, synthetic construct
EVQLVESGGGLVQPGGSLRLSCAASGFTFIGYSMNWVRQAPGKGLEWVSYISSYESTIYYADSVKGRFTISRDNAKNSLYLQMNSLRAEDTAVYYCARSRITLVRGVPRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGSGGGGSGGGGSGGGGSEVQLLESGGGLVQPGGSLRLSCAASGFTFSSRGMSWVRQAPGKCLEWVSFISSSGTYVYYADSVKGRFTISRDNSKNTLYLQMNSLRAEDTAVYYCARDLPRTGVYFGMDVWGQGTTVTVSSGGGGSGGGGSGGGGSGGGGSDIQMTQSPSSLSASVGDRVTITCRASQSIFNYLNWYQQKPGKAPKLLIYGASTLQSGVPSRFSGSGSGTDFTLTISSLQPEDFATYYCQESYSTPYTFGCGTKVEIK

(19) United States
(12) Patent Application Publication
(10) Pub. No.: US 2025/0376536 A1
(43) Pub. Date: Dec. 11, 2025

(54) MULTISPECIFIC ANTI-CD40 / ANTI-FAP ANTIBODIES AND USES THEREOF

(71) Applicant: Bristol-Myers Squibb Company, Princeton, NJ (US)

(72) Inventors: Haralambos Antonis HADJIVASSILIOU, San Diego, CA (US); Tessie Man Wai NG, San Francisco, CA (US); Christopher Mark HILL, Seattle, WA (US); Funminiyi Samuel BAMIDELE, San Diego, CA (US); Justine Kamilah WALKER, San Diego, CA (US); Neelu Hariram YADAV, Williamsville, NY (US); Xiaogang HAN, Lexington, MA (US); Mayu MIYAMURA, Fort Lee, NJ (US); Brian Forrest KIESEL, Summit, NJ (US)

(73) Assignee: Bristol-Myers Squibb Company, Princeton, NJ (US)

(21) Appl. No.: 19/229,815

(22) Filed: Jun. 5, 2025

Related U.S. Application Data
(60) Provisional application No. 63/656,769, filed on Jun. 6, 2024.
(60) Provisional application No. 63/676,045, filed on Jul. 26, 2024.

(51) Int. Cl. C07K 16/28; A61P 35/04; C07K 16/40

(52) U.S. Cl. CPC C07K 16/2878; A61P 35/04; C07K 16/40; C07K 2317/31; C07K 2317/51; C07K 2317/515; C07K 2317/522; C07K 2317/55; C07K 2317/565; C07K 2317/622

(57) ABSTRACT

The present application relates to antibodies (e.g., multispecific antibodies) that bind to CD40 and/or FAP, as well as nucleic acids encoding the antibodies, vectors and host cells comprising the nucleic acids, and methods of producing and using the antibodies.

### CROSS-REFERENCE TO RELATED APPLICATIONS

This application claims priority from U.S. provisional application No. 63/656,769, filed Jun. 6, 2024, entitled “MULTISPECIFIC ANTI-CD40/ANTI-FAP ANTIBODIES AND USES THEREOF” and U.S. provisional application No. 63/676,045, filed Jul. 26, 2024, entitled “MULTISPECIFIC ANTI-CD40/ANTI-FAP ANTIBODIES AND USES THEREOF”, the contents of each of which are incorporated by reference in their entireties.

### INCORPORATION BY REFERENCE OF SEQUENCE LISTING

The present application is being filed along with a Sequence Listing in electronic format. The contents of the electronic sequence listing (217372000800SEQLIST.xml; Size: 58,597 bytes; and Date of Creation: Jun. 4, 2025) is herein incorporated by reference in its entirety.

### FIELD

The present application relates to multispecific antibodies that bind CD40 and FAP, nucleic acids encoding the antibodies, vectors and host cells comprising the nucleic acids, and methods of making and using the antibodies. The present application also relates to antibodies that bind CD40, nucleic acids encoding the antibodies, vectors and host cells comprising the nucleic acids, and methods of making and using the antibodies.

### BACKGROUND

CD40 is a costimulatory receptor that is expressed on DCs and macrophages among other cell types. Binding to its endogenous ligand, CD40L, triggers the TRAF family of adaptor proteins to activate NFκB, MAPK, and other intracellular signalling cascades to upregulate pro-inflammatory transcriptional programs (Elgueta et al. Immunol Rev, 2009; 229:152-72). Unique to DCs, CD40 activation “licenses” them to present tumor antigens to CD8 T cells that, in turn, initiates CD8 T-cell cytotoxic capabilities (Bennett et al. Nature, 1998; 393:478-80; Schoenberger et al. Nature, 1998; 393:480-3; Ridge et al. Nature, 1998; 393:474-8). Additionally, CD40 agonism reprograms suppressive macrophages to further augment the anti-tumor immunity cycle through recruitment, priming, and activation of CD4 T cells (Byrne et al. Cell Rep, 2016; 15:2719-3; Huffman et al. JCI Insight, 2020; 5: e137263; Patterson et al. Cell Rep, 2023; 42:112732). Taken together, CD40 serves as an important node to drive innate and adaptive immune functions toward tumor growth inhibition.

FAP is expressed at low levels in healthy adult tissue with the exception of wound healing due to its primary role in tissue remodelling (Xin et al. Front Oncol, 2021; 11:648187). In tumor stroma, FAP is upregulated on cancer-associated fibroblasts across multiple solid tumor indications including pancreatic, gastric, lung, colorectal, and others. FAP enrichment in solid tumors has also been demonstrated using FAP positron-emitting radiopharmaceutical tracers in cancer patients (Sharma P et al. Cell, 2017; 168 (4): 707-723; Schadendorf et al. J Clin Oncol, 2017; 35 (34): 3807-3814).

Immune-modulating therapies have demonstrated improved outcomes for patients with various types of advanced solid malignancies, with agents targeting the programmed cell death 1 (PD-1) and cytotoxic T lymphocyte-associated antigen 4 (CTLA-4) pathways being extensively studied. However, despite the clinical success of these agents, many patients either do not respond or experience a limited duration of response.

There exists a need for safe and effective agents and methods for treating, preventing and managing cancer, including for cancers that are refractory to standard treatments, while reducing or avoiding the toxicities and/or side effects associated with some existing therapies.

### SUMMARY

The present disclosure relates to multispecific antibodies that bind CD40 and FAP, nucleic acids encoding the antibodies, vectors and host cells comprising the nucleic acids, and methods of both making and using the antibodies. The present disclosure also relates to antibodies that bind CD40, nucleic acids encoding the antibodies, vectors and host cells comprising the nucleic acids, and methods of both making and using the antibodies. For example, embodiments of the disclosure include the following:

Embodiment 1. A multispecific antibody comprising a first antigen binding domain that binds CD40, a second antigen binding domain that binds CD40, and a third antigen binding domain that binds fibroblast activation protein alpha (FAP), wherein the third antigen binding domain is a single chain variable region (scFv).

Embodiment 2. The multispecific antibody of embodiment 1, wherein the first antigen binding domain comprises a first heavy chain variable region and a first light chain variable region, the second antigen binding domain comprises a second heavy chain variable region and a second light chain variable region, and the scFv comprises a third heavy chain variable region and a third light chain variable region.

Embodiment 3. The multispecific antibody of embodiment 2, wherein the multispecific antibody comprises:
a) a first heavy chain comprising the first heavy chain variable region fused to a first heavy chain constant region;
b) a first light chain comprising the first light chain variable region fused to a first light chain constant region;
c) a second heavy chain comprising the second heavy chain variable region fused to a second heavy chain constant region; and
d) a second light chain comprising the second light chain variable region fused to a second light chain constant region;
   wherein the first heavy chain constant region and the second heavy chain constant region are the same or different.

Embodiment 4. The multispecific antibody of embodiment 3, wherein the scFv is fused to the C-terminus of the first heavy chain constant region.

Embodiment 5. The multispecific antibody of embodiment 3 or embodiment 4, wherein the first heavy chain constant region and the second heavy chain constant region are different.

Embodiment 6. The multispecific antibody of embodiment 3 or embodiment 4, wherein
each of the first heavy chain constant region and the second heavy chain constant region comprises a heavy chain constant region 1 (CH1) and a Fc polypeptide chain, and
   wherein the first binding domain and second binding domain each comprise a Fab, wherein each Fab comprises:
      (i) a Fab heavy chain comprising the first binding domain or second variable heavy chain and the CH1, and
      (ii) the first or second light chain, wherein each Fab is the same.
   Embodiment 7. The multispecific antibody of embodiment 6, wherein each CH1 is an IgG1 CH1.

Embodiment 8. The multispecific antibody of embodiment 6 or embodiment 7, wherein each CH1 comprises the amino acid sequence set forth in SEQ ID NO: 50.

Embodiment 9. The multispecific antibody of any one of embodiments 6-8, wherein the Fc polypeptide chain of the first and second heavy chain constant region are different.

Embodiment 10. The multispecific antibody of any one of embodiments 3-9, wherein the first heavy chain constant region and the second heavy chain constant region form a heterodimer.

Embodiment 11. The multispecific antibody of embodiment 5 or embodiment 6, wherein the Fc polypeptide chain of the first heavy chain constant region comprises at least one first heterodimerization mutation and the Fc polypeptide chain of the second heavy chain constant region comprises at least one second heterodimerization mutation.

Embodiment 12. The multispecific antibody of any one of embodiments 3-11, wherein the first heavy chain constant region comprises at least one first heterodimerization mutation and the second heavy chain constant region comprises at least one second heterodimerization mutation.

Embodiment 13. The multispecific antibody of embodiment 11 or embodiment 12, wherein the at least one first heterodimerization mutation and at least one second heterodimerization mutation are with reference to an Fc polypeptide chain of an IgG1 constant region.

Embodiment 14. The multispecific antibody of any one of embodiments 11-13, wherein at least one first heterodimerization mutation comprises T366W and at least one second heterodimerization mutation comprises one or more of T366S, L368A, and/or Y407V, or wherein at least one first heterodimerization mutation comprises one or more of T366S, L368A, and/or Y407V and at least one second heterodimerization mutation comprises T366W, wherein mutation positions are according to Kabat.

Embodiment 15. The multispecific antibody of any one of embodiments 11-13, wherein at least one first heterodimerization mutation comprises T366W and at least one second heterodimerization mutation comprises T366S, L368A, and Y407V, or wherein at least one first heterodimerization mutation comprises T366S, L368A, and Y407V and at least one second heterodimerization mutation comprises T366W, wherein mutation positions are according to Kabat.

Embodiment 16. The multispecific antibody of any one of embodiments 11-13, wherein at least one first heterodimerization mutation comprises one or more of T350V, L351Y, S400E, F405A, and/or Y407V and at least one second heterodimerization mutation comprises one or more of T350V, T366L, N390R, K392M, K392L, and/or T394W, or wherein at least one first heterodimerization mutation comprises one or more of T350V, T366L, N390R, K392M, K392L, and/or T394W and at least one second heterodimerization mutation comprises one or more of T350V, L351Y, S400E, F405A, and/or Y407V, wherein mutation positions are according to Kabat.

Embodiment 17. The multispecific antibody of any one of embodiments 11-13, wherein at least one first heterodimerization mutation comprises T350V, L351Y, F405A, and Y407V and at least one second heterodimerization mutation comprises T350V, T366L, K392L, and T394W, or wherein at least one first heterodimerization mutation comprises one or more of T350V, T366L, K392L, and T394W and at least one second heterodimerization mutation comprises one or more of T350V, L351Y, F405A, and Y407V, wherein mutation positions are according to Kabat.

Embodiment 18. The multispecific antibody of any one of embodiments 3-17, wherein the first heavy chain constant region and the second heavy chain constant region are IgG1 constant regions.

Embodiment 19. The multispecific antibody of any one of embodiments 3-18, wherein the first heavy chain constant region and the second heavy chain constant region do not bind FcγR or have reduced binding to FcγR compared to wild-type constant regions of the same isotype.

Embodiment 20. The multispecific antibody of any one of embodiments 6-19, wherein each Fc polypeptide chain is effectorless.

Embodiment 21. The multispecific antibody of any one of embodiments 6-20, wherein each Fc polypeptide chain comprises L234A, L235A, and/or D265S substitutions, wherein substitution positions are according to Kabat.

Embodiment 22. The multispecific antibody of any one of embodiments 6-21, wherein the Fc polypeptide chain of the first heavy chain constant region comprises an amino acid sequence at least 85%, at least 90%, or at least 95% identical to the amino acid sequence of SEQ ID NO: 51 or SEQ ID NO: 52, and wherein the Fc polypeptide chain of the second heavy chain constant region comprises an amino acid sequence at least 85%, at least 90%, or at least 95% identical to the amino acid sequence of the other of SEQ ID NO: 51 or SEQ ID NO: 52.

Embodiment 23. The multispecific antibody of any one of embodiments 6-22, wherein the Fc polypeptide chain of the first heavy chain constant region comprises the amino acid sequence of SEQ ID NO: 51 or SEQ ID NO: 52, and wherein the Fc polypeptide chain of the second heavy chain constant region comprises the amino acid sequence of the other of SEQ ID NO: 51 or SEQ ID NO: 52.

Embodiment 24. The multispecific antibody of any one of embodiments 6-22, wherein the Fc polypeptide chain of the first heavy chain constant region comprises the amino acid sequence of SEQ ID NO: 55 or SEQ ID NO: 56, and wherein the Fc polypeptide chain of the second heavy chain constant region comprises the amino acid sequence of the other of SEQ ID NO: 55 or SEQ ID NO: 56.

Embodiment 25. The multispecific antibody of any one of embodiments 1-24, wherein the first antigen binding domain comprises a first heavy chain variable region (VH) and a first light chain variable region (VL), wherein:
a) the first heavy chain variable region comprises a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and
b) the first light chain variable region comprises a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

Embodiment 26. The multispecific antibody of embodiment 25, wherein the first heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the first light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

Embodiment 27. The multispecific antibody of embodiment 25, wherein the first heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the first light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

Embodiment 28. The multispecific antibody of any one of embodiments 25-27, wherein the first heavy chain variable region comprises an amino acid sequence at least 85%, at least 90%, or at least 95% identical to the amino acid sequence of SEQ ID NO: 13; and the first light chain variable region comprises an amino acid sequence at least 85%, at least 90%, or at least 95% identical to the amino acid sequence of SEQ ID NO: 14.

Embodiment 29. The multispecific antibody of any one of embodiments 1-28, wherein the first antigen binding domain comprises a first heavy chain variable region comprising the amino acid sequence of SEQ ID NO: 13, and a first light chain variable region comprising the amino acid sequence of SEQ ID NO: 14.

Embodiment 30. The multispecific antibody of any one of embodiments 1-29, wherein the second antigen binding domain comprises a second heavy chain variable region and a second light chain variable region, wherein:
a) the second heavy chain variable region comprises a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and
b) the second light chain variable region comprises a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

Embodiment 31. The multispecific antibody of embodiment 30, wherein the second heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the second light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

Embodiment 32. The multispecific antibody of embodiment 30, wherein the second heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the second light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

Embodiment 33. The multispecific antibody of any one of embodiments 30-32, wherein the second heavy chain variable region comprises an amino acid sequence at least 85%, at least 90%, or at least 95% identical to the amino acid sequence of SEQ ID NO: 13; and the second light chain variable region comprises an amino acid sequence at least 85%, at least 90%, or at least 95% identical to the amino acid sequence of SEQ ID NO: 14.

Embodiment 34. The multispecific antibody of any one of embodiments 1-33, wherein the second antigen binding domain comprises a second heavy chain variable region comprising the amino acid sequence of SEQ ID NO: 13, and a second light chain variable region comprising the amino acid sequence of SEQ ID NO: 14.

Embodiment 35. The multispecific antibody of any one of embodiments 6-34, wherein each Fab heavy chain comprises an amino acid sequence at least 85%, at least 90%, or at least 95% identical to the amino acid sequence of SEQ ID NO: 53.

Embodiment 36. The multispecific antibody of any one of embodiments 6-35, wherein each Fab heavy chain comprises the amino acid sequence of SEQ ID NO: 53.

Embodiment 37. The multispecific antibody of any one of embodiments 1-36, wherein the scFv comprises a third heavy chain variable region and a third light chain variable region, wherein:
a) the third heavy chain variable region comprises a heavy chain complementarity determining region (HCDR) 1 comprising the amino acid sequence of SEQ ID NO: 17 or 23, a HCDR2 comprising the amino acid sequence of SEQ ID NO: 18 or 24, and a HCDR3 comprising the amino acid sequence of SEQ ID NO: 19 or 25; and
b) the third light chain variable region comprises a LCDR1 comprising the amino acid sequence of SEQ ID NO: 20 or 26, a LCDR2 comprising the amino acid sequence of SEQ ID NO: 21 or 27, and a LCDR3 comprising the amino acid sequence of SEQ ID NO: 22 or 28.

Embodiment 38. The multispecific antibody of embodiment 37, wherein the third heavy chain variable region comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19; and the third light chain variable region comprises a LCDR1 comprising the amino acid sequence of SEQ ID NO: 20, a LCDR2 comprising the amino acid sequence of SEQ ID NO: 21, and a LCDR3 comprising the amino acid sequence of SEQ ID NO: 22.

Embodiment 39. The multispecific antibody of embodiment 37, wherein the third heavy chain variable region comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25; and the third light chain variable region comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

Embodiment 40. The multispecific antibody of any one of embodiments 37-39, wherein the third heavy chain variable region comprises an amino acid sequence at least 85%, at least 90%, or at least 95% identical to the amino acid sequence of SEQ ID NO: 29 or 31; and the third light chain variable region comprises an amino acid sequence at least 85%, at least 90%, or at least 95% identical to the amino acid sequence of SEQ ID NO: 30 or 32.

Embodiment 41. The multispecific antibody of any one of embodiments 1-40, wherein the third antigen binding domain comprises a third heavy chain variable region comprising the amino acid sequence of SEQ ID NO: 29 or 31; and a third light chain variable region comprising the amino acid sequence of SEQ ID NO: 30 or 32.

Embodiment 42. The multispecific antibody of any one of embodiments 1-41, wherein the third antigen binding domain comprises a third heavy chain variable region comprising the amino acid sequence of SEQ ID NO: 29; and a third light chain variable region comprising the amino acid sequence of SEQ ID NO: 30.

Embodiment 43. The multispecific antibody of any one of embodiments 1-41, wherein the third antigen binding domain comprises a third heavy chain variable region comprises the amino acid sequence of SEQ ID NO: 31; and a third light chain variable region comprising the amino acid sequence of SEQ ID NO: 32.

Embodiment 44. The multispecific antibody of any one of embodiments 1-43, wherein the third antigen binding domain is an scFv comprising the amino acid sequence of SEQ ID NO: 33.

Embodiment 45. The multispecific antibody of any one of embodiments 6-44, wherein the multispecific antibody comprises:
a) the first and second antigen binding domains that binds CD40, and each comprises the Fab; and
b) a heterodimeric Fc region comprising the Fc polypeptide chain of the first heavy chain constant region and the Fc polypeptide chain of the second heavy chain constant region, where the Fc polypeptide chain of the first heavy chain constant region and Fc polypeptide chain of the second heavy chain constant region are each linked to the C-terminus of one of the Fab heavy chains and are linked to different Fab heavy chains; and
b) the scFv of the third binding domain that is fused to the C-terminus of the Fc polypeptide chain of the first heavy chain constant region.

Embodiment 46. The multispecific antibody of any one of embodiments 6-45, wherein the Fab comprises the Fab heavy chain comprising the amino acid sequence of SEQ ID NO: 53 and the first light chain comprising the amino acid sequence of SEQ ID NO: 16.

Embodiment 47. The multispecific antibody of any one of embodiments 6-46, wherein the multispecific antibody comprises a first polypeptide, a second polypeptide, and a third polypeptide, wherein:
a) the first polypeptide comprises, from N-terminus to C-terminus: the Fab heavy chain of the first binding domain, the Fc polypeptide chain of the first heavy chain constant region, and the scFv of the third binding domain;
b) the second polypeptide comprises, from N-terminus to C-terminus: the Fab heavy chain of the second binding domain and the Fc polypeptide region of the second heavy chain constant region; and
c) the third polypeptide comprises the first light chain.

Embodiment 48. The multispecific antibody of any one of embodiments 6-47, wherein the multispecific antibody comprises a first polypeptide, a second polypeptide, and a third polypeptide, wherein:
a) the first polypeptide comprises, from N-terminus to C-terminus: the Fab heavy chain of the first binding domain comprising the amino acid sequence of SEQ ID NO: 53, the Fc polypeptide chain of the first heavy chain constant region comprising the amino acid sequence of SEQ ID NO: 52, and the scFv of the third binding domain comprising the amino acid sequence of SEQ ID NO: 33;
b) the second polypeptide comprises, from N-terminus to C-terminus: the Fab heavy chain of the second binding domain comprising the amino acid sequence of SEQ ID NO: 53 and the Fc polypeptide chain of the second heavy chain constant region comprising the amino acid sequence of SEQ ID NO: 51; and
c) the third polypeptide comprises the first light chain comprising the amino acid sequence of SEQ ID NO: 16.

Embodiment 49. The multispecific antibody of any one of embodiments 6-47, wherein the multispecific antibody comprises a first polypeptide, a second polypeptide, and a third polypeptide, wherein:
a) the first polypeptide comprises, from N-terminus to C-terminus: the Fab heavy chain of the first binding domain comprising the amino acid sequence of SEQ ID NO: 53, the Fc polypeptide chain of the first heavy chain constant region comprising the amino acid sequence of SEQ ID NO: 56, and the scFv of the third binding domain comprising the amino acid sequence of SEQ ID NO: 33;
b) the second polypeptide comprises, from N-terminus to C-terminus: the Fab heavy chain of the second binding domain comprising the amino acid sequence of SEQ ID NO: 53 and the Fc polypeptide chain of the second heavy chain constant region comprising the amino acid sequence of SEQ ID NO: 55;
c) the third polypeptide comprises the first light chain comprising the amino acid sequence of SEQ ID NO: 16.

Embodiment 50. The multispecific antibody of any one of embodiments 1-49, wherein the multispecific antibody comprises a first polypeptide, a second polypeptide, and a third polypeptide, wherein:
a) the first polypeptide comprises, from N-terminus to C-terminus: a first heavy chain variable region, a first heavy chain constant region and an scFv comprising a third heavy chain variable region and a third light chain variable region;
b) the second polypeptide comprises, from N-terminus to C-terminus: a second heavy chain variable region and a second heavy chain constant region; and
c) the third polypeptide comprises a first light chain variable region and a first light chain constant region;
   wherein the ratio of the first polypeptide to the second polypeptide to the third polypeptide is 1:1:2.

Embodiment 51. The multispecific antibody of any one of embodiments 47-50, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Embodiment 52. The multispecific antibody of any one of embodiments 47-51, comprising at least one post-translational modification of the first polypeptide, second polypeptide, and/or third polypeptide.

Embodiment 53. The multispecific antibody of any one of embodiments 47-50 and 52, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

Embodiment 54. The multispecific antibody of any one of embodiments 47-50 and 52, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

Embodiment 55. The multispecific antibody of any one of embodiments 47-50 and 52, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

Embodiment 56. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Embodiment 57. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

Embodiment 58. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

Embodiment 59. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

Embodiment 60. The multispecific antibody of any one of embodiments 1-59, wherein the multispecific antibody is a CD40 agonist in the presence of FAP-expressing cells.

Embodiment 61. The multispecific antibody of any one of embodiments 1-60, wherein the multispecific antibody activates dendritic cells and macrophages in the presence of FAP-expressing cells.

Embodiment 62. The multispecific antibody of any one of embodiments 1-61, wherein the multispecific antibody does not compete with CD40L for binding to CD40.

Embodiment 63. The multispecific antibody of any one of embodiments 1-61, wherein the multispecific antibody binds CD40 with a K_{D} between 20 and 200 nM, or 50 and 200 nM, or 50 and 150 nM, as determined by surface plasmon resonance.

Embodiment 64. The multispecific antibody of any one of embodiments 1-61, wherein the multispecific antibody binds CD40 expressed on the surface of cells with an EC50 of 1-50 nM, or 1-25 nM, or 3-20 nM, or 3-15 nM.

Embodiment 65. The multispecific antibody of any one of embodiments 1-64, wherein the multispecific antibody binds FAP with a K_{D} between 0.5 and 10 nM, or 0.5 and 7 nM, or 1 and 5 nM, as determined by surface plasmon resonance.

Embodiment 66. The multispecific antibody of any one of embodiments 1-64, wherein the multispecific antibody binds FAP expressed on the surface of cells with an EC50 of between 0.5 and 10 nM, or 0.5 and 7 nM, or 1 and 5 nM.

Embodiment 67. A pharmaceutical composition comprising the multispecific antibody of any one of embodiments 1-66 and a pharmaceutically acceptable carrier.

Embodiment 68. An isolated nucleic acid that encodes the multispecific antibody of any one of embodiments 1-66.

Embodiment 69. The isolated nucleic acid of embodiment 68, which is an expression vector.

Embodiment 70. An isolated nucleic acid that encodes the first polypeptide, the second polypeptide, and/or the third polypeptide of the multispecific antibody of any one of embodiments 47-66.

Embodiment 71. The isolated nucleic acid of embodiment 70, which is an expression vector.

Embodiment 72. A host cell that expresses the multispecific antibody of any one of embodiments 1-66.

Embodiment 73. A host cell comprising the nucleic acid of embodiment 68 or embodiment 69.

Embodiment 74. A host cell comprising the nucleic acid of embodiment 70 or embodiment 71.

Embodiment 75. A host cell comprising a first polynucleotide sequence that encodes the first polypeptide, a second polynucleotide sequence that encodes the second polypeptide, and a third nucleic acid sequence that encodes the third polypeptide, of the multispecific antibody of any one of embodiments 47-66.

Embodiment 76. A method of producing a multispecific antibody comprising culturing the host cell of any one of embodiments 72-75 under conditions suitable for expressing the multispecific antibody.

Embodiment 77. The method of embodiment 76, further comprising isolating the multispecific antibody.

Embodiment 78. An antibody or antigen-binding fragment thereof comprising an antigen binding domain that binds CD40, wherein the antigen binding domain comprises a heavy chain variable region comprising a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and a light chain variable region comprising a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

Embodiment 79. The antibody or antigen-binding fragment thereof of embodiment 78, wherein the heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

Embodiment 80. The antibody or antigen-binding fragment thereof of embodiment 78, wherein the heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

Embodiment 81. The antibody or antigen-binding fragment thereof of any one of embodiments 78-80, wherein the heavy chain variable region comprises an amino acid sequence at least 85%, at least 90%, or at least 95% identical to the amino acid sequence of SEQ ID NO: 13; and the light chain variable region comprises an amino acid sequence at least 85%, at least 90%, or at least 95% identical to the amino acid sequence of SEQ ID NO: 14.

Embodiment 82. The antibody or antigen-binding fragment thereof of any one of embodiments 78-81, wherein the heavy chain variable region comprises the amino acid sequence of SEQ ID NO: 13, and the light chain variable region comprises the amino acid sequence of SEQ ID NO: 14.

Embodiment 83. The antibody or antigen-binding fragment thereof of any one of embodiments 78-82, wherein the antibody or antigen-binding fragment thereof is a bispecific antibody.

Embodiment 84. The antibody or antigen-binding fragment thereof of embodiment 83, wherein the antibody or antigen-binding fragment thereof further comprises a second antigen binding domain that binds FAP.

Embodiment 85. The antibody or antigen-binding fragment thereof of embodiment 84, wherein the second antigen binding domain comprises a second heavy chain variable region comprising a heavy chain complementarity determining region (HCDR) 1 comprising the amino acid sequence of SEQ ID NO: 17 or 23, a HCDR2 comprising the amino acid sequence of SEQ ID NO: 18 or 24, and a HCDR3 comprising the amino acid sequence of SEQ ID NO: 19 or 25, and a second light chain variable region comprising a LCDR1 comprising the amino acid sequence of SEQ ID NO: 20 or 26, a LCDR2 comprising the amino acid sequence of SEQ ID NO: 21 or 27, and a LCDR3 comprising the amino acid sequence of SEQ ID NO: 22 or 28.

Embodiment 86. The antibody or antigen-binding fragment thereof of embodiment 84, wherein the second heavy chain variable region comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19; and the second light chain variable region comprises a LCDR1 comprising the amino acid sequence of SEQ ID NO: 20, a LCDR2 comprising the amino acid sequence of SEQ ID NO: 21, and a LCDR3 comprising the amino acid sequence of SEQ ID NO: 22.

Embodiment 87. The antibody or antigen-binding fragment thereof of embodiment 84, wherein the second heavy chain variable region comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25; and the second light chain variable region comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

Embodiment 88. The antibody or antigen-binding fragment thereof of any one of embodiments 85-87, wherein the second heavy chain variable region comprises an amino acid sequence at least 85%, at least 90%, or at least 95% identical to the amino acid sequence of SEQ ID NO: 29 or 31; and the second light chain variable region comprises an amino acid sequence at least 85%, at least 90%, or at least 95% identical to the amino acid sequence of SEQ ID NO: 30 or 32.

Embodiment 89. The antibody or antigen-binding fragment thereof of any one of embodiments 84-88, wherein the second antigen binding domain comprises a second heavy chain variable region comprising the amino acid sequence of SEQ ID NO: 29 or 31; and a second light chain variable region comprising the amino acid sequence of SEQ ID NO: 30 or 32.

Embodiment 90. The antibody or antigen-binding fragment thereof of any one of embodiments 84-88, wherein the second antigen binding domain comprises a second heavy chain variable region comprising the amino acid sequence of SEQ ID NO: 29; and a second light chain variable region comprising the amino acid sequence of SEQ ID NO: 30.

Embodiment 91. The antibody or antigen-binding fragment thereof of any one of embodiments 84-88, wherein the second antigen binding domain comprises a second heavy chain variable region comprises the amino acid sequence of SEQ ID NO: 31; and a second light chain variable region comprising the amino acid sequence of SEQ ID NO: 32.

Embodiment 92. The antibody or antigen-binding fragment thereof of any one of embodiments 84-91, wherein the second antigen binding domain is a Fab, Fab′, F(ab′)₂, Fd, Fv, single-chain Fv (scFv) or disulfide-linked Fv (sdFv).

Embodiment 93. The antibody or antigen-binding fragment thereof of any one of embodiments 84-92, wherein the second antigen binding domain is a scFv.

Embodiment 94. The antibody or antigen-binding fragment thereof of embodiment 92 or embodiment 93, wherein the scFv comprised the amino acid sequence of SEQ ID NO: 33.

Embodiment 95. The antibody or antigen-binding fragment thereof of any one of embodiments 78-94, wherein the antibody comprises a third antigen binding domain that binds CD40.

Embodiment 96. The antibody or antigen-binding fragment thereof of embodiment 95, wherein the first antigen binding domain and the third antigen binding domain are the same or different.

Embodiment 97. A pharmaceutical composition comprising the antibody or antigen-binding fragment thereof of any one of embodiments 78-96 and a pharmaceutically acceptable carrier.

Embodiment 98. An isolated nucleic acid that encodes the antibody or antigen-binding fragment thereof of any one of embodiments 78-96.

Embodiment 99. The isolated nucleic acid of embodiment 98, which is an expression vector.

Embodiment 100. A host cell that expresses the antibody of any one of embodiments 78-96.

Embodiment 101. A host cell comprising the nucleic acid of embodiment 98 or embodiment 99.

Embodiment 102. A method of producing an antibody or antigen-binding fragment thereof comprising culturing the host cell of embodiment 100 or embodiment 101 under conditions suitable for expressing the antibody.

Embodiment 103. The method of embodiment 102, further comprising isolating the antibody or antigen-binding fragment thereof.

Embodiment 104. A method of treating cancer comprising administering to a subject in need thereof the multispecific antibody of any one of embodiments 1-66, the antibody or antigen-binding fragment thereof of any one of embodiments 78-96, or the pharmaceutical composition of embodiment 67 or embodiment 97.

Embodiment 105. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Embodiment 106. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

Embodiment 107. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

Embodiment 108. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

Embodiment 109. The method of any one of embodiments 104-108, wherein the cancer is a solid tumor.

Embodiment 110. The method of any one of embodiments 104-109, wherein the cancer is gastric cancer or pancreatic cancer.

Embodiment 111. The method of embodiment 110 wherein the pancreatic cancer is pancreatic ductal adenocarcinoma.

Embodiment 112. The method of any one of embodiments 104-110, wherein the cancer is selected from non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN).

Embodiment 113. The method of any one of embodiments 104-112, wherein the cancer is advanced unresectable cancer.

Embodiment 114. The method of any one of embodiments 104-113, wherein the cancer is metastatic cancer.

Embodiment 115. The method of any one of embodiments 104-114, wherein the cancer is recurrent cancer.

Embodiment 116. The method of any one of embodiments 104-115, comprising administering to a subject about 10 mg to about 1000 mg of the multispecific antibody.

Embodiment 117. The method of embodiment 116, comprising administering to a subject about 10 mg of the multispecific antibody.

Embodiment 118. The method of embodiment 116, comprising administering to a subject about 30 mg of the multispecific antibody.

Embodiment 119. The method of embodiment 116, comprising administering to a subject about 90 mg of the multispecific antibody.

Embodiment 120. The method of embodiment 116, comprising administering to a subject about 250 mg of the multispecific antibody.

Embodiment 121. The method of embodiment 116, comprising administering to a subject about 500 mg of the multispecific antibody.

Embodiment 122. The method of embodiment 116, comprising administering to a subject about 1000 mg of the multispecific antibody.

Embodiment 123. The method of any one of embodiments 104-122, wherein the multispecific antibody is administered in a cycling regimen of one or more 21-day or 28-day cycles, wherein Day 1 is the first day of each cycle.

Embodiment 124. The method of any one of embodiments 104-123, wherein the multispecific antibody is administered to the subject about once every week, about once every two weeks, about once every three weeks, or about once every four weeks.

Embodiment 125. The method of embodiment 124, wherein the multispecific antibody is administered to the subject about once every two weeks.

Embodiment 126. The method of embodiment 124, wherein the multispecific antibody is administered to the subject about once every three weeks.

Embodiment 127. The method of any one of embodiments 104-126, wherein the multispecific antibody is administered to the subject by intravenous administration.

Embodiment 128. The method of any one of embodiments 104-126, wherein the multispecific antibody is administered to the subject by subcutaneous administration.

Embodiment 129. The method of any one of embodiments 104-128, wherein the method further comprises administering at least one PD-1 therapy.

Embodiment 130. The method of embodiment 129, wherein the PD-1 therapy is an antibody or antigen-binding fragment thereof that binds to PD-1, or an antibody or antigen-binding fragment thereof that binds to PD-L1.

Embodiment 131. The method of embodiment 129 or embodiment 130, wherein the multispecific antibody and the PD-1 therapy are administered separately.

Embodiment 132. The method of embodiment 131, wherein the multispecific antibody and PD-1 therapy are administered sequentially.

Embodiment 133. The method of embodiment 132, wherein the multispecific antibody is administered after the PD-1 therapy.

Embodiment 134. The method of embodiment 132, wherein the PD-1 therapy is administered after the multispecific antibody.

Embodiment 135. The method of embodiment 131, wherein the multispecific antibody and PD-1 therapy are co-administered, wherein the multispecific antibody is in a first bag and the PD-1 therapy is in a second bag, and wherein the multispecific antibody and the PD-1 therapy are administered simultaneously.

Embodiment 136. The method of any one of embodiments 129-135, wherein the PD-1 therapy is administered to the subject by intravenous administration.

Embodiment 137. The method of any one of embodiments 129-136, wherein the PD-1 therapy is administered in a cycling regimen of one or more 21-day or 28-day cycles, wherein Day 1 is the first day of each cycle.

Embodiment 138. The method of any one of embodiments 129-137, wherein the PD-1 therapy is selected from nivolumab, pembrolizumab, cemiplimab, dostarlimab, retifanlimab, toripalimab, atezolizumab, avelumab, and durvalumab.

Embodiment 139. The method of any one of embodiments 129-138, wherein the PD-1 therapy is nivolumab.

Embodiment 140. The method of embodiment 138 or embodiment 139, wherein nivolumab is administered to the subject about once every two weeks at a dose of 240 mg, about once every three weeks at a dose of 360 mg, or about once every four weeks at a dose of 480 mg.

Embodiment 141. The method of embodiment 138 or embodiment 139, wherein nivolumab is administered to the subject about once every two weeks at a dose of 240 mg.

Embodiment 142. The method of embodiment 138 or embodiment 139, wherein nivolumab is administered to the subject about once every two weeks at a dose of 600 mg.

Embodiment 143. The method of embodiment 138 or embodiment 139, wherein nivolumab is administered to the subject about once every two weeks at a dose of 720 mg.

Embodiment 144. The method of embodiment 138 or embodiment 139, wherein nivolumab is administered to the subject about once every two weeks at a dose of 960 mg.

Embodiment 145. The method of embodiment 138 or embodiment 139, wherein nivolumab is administered to the subject about once every two weeks at a dose of 1200 mg.

Embodiment 146. The method of embodiment 138 or embodiment 139, wherein nivolumab is administered to the subject about once every three weeks at a dose of 360 mg.

Embodiment 147. The method of embodiment 138 or embodiment 139, wherein nivolumab is administered to the subject about once every three weeks at a dose of 720 mg.

Embodiment 148. The method of embodiment 138 or embodiment 139, wherein nivolumab is administered to the subject about once every three weeks at a dose of 900 mg.

Embodiment 149. The method of embodiment 138 or embodiment 139, wherein nivolumab is administered to the subject about once every three weeks at a dose of 960 mg.

Embodiment 150. The method of embodiment 138 or embodiment 139, wherein nivolumab is administered to the subject about once every three weeks at a dose of 1200 mg.

Embodiment 151. The method of embodiment 138 or embodiment 139, wherein nivolumab is administered to the subject about once every four weeks at a dose of 480 mg.

Embodiment 152. The method of embodiment 138 or embodiment 139, wherein nivolumab is administered to the subject about once every four weeks at a dose of 720 mg.

Embodiment 153. The method of embodiment 138 or embodiment 139, wherein nivolumab is administered to the subject about once every four weeks at a dose of 960 mg.

Embodiment 154. The method of embodiment 138 or embodiment 139, wherein nivolumab is administered to the subject about once every four weeks at a dose of 1200 mg.

Embodiment 155. The method of any one of embodiments 138-154, wherein nivolumab is administered intravenously.

Embodiment 156. The method of any one of embodiments 138-154, wherein nivolumab is administered subcutaneously.

Embodiment 157. The method of any one of embodiments 138-154 and 156, wherein nivolumab is co-formulated with hyaluronidase.

Embodiment 158. The method of embodiment 157, wherein hyaluronidase is at a dose of about 10,000 units to about 20,000 units, inclusive.

Embodiment 159. The method of embodiment 157 or embodiment 158, wherein hyaluronidase is at a dose of about 10,000 units; about 12,000 units; 15,000 units; about 16,000 units; or about 20,000 units.

Embodiment 160. The method of any one of embodiments 157-159, wherein nivolumab co-formulated with hyaluronidase is administered to the subject about once every two weeks at a dose of 600 mg nivolumab and a dose of about 10,000 units hyaluronidase.

Embodiment 161. The method of any one of embodiments 157-159, wherein nivolumab co-formulated with hyaluronidase is administered to the subject about once every three weeks at a dose of 900 mg nivolumab and a dose of about 15,000 units hyaluronidase.

Embodiment 162. The method of any one of embodiments 157-159, wherein nivolumab co-formulated with hyaluronidase is administered to the subject about once every four weeks at a dose of 1200 mg nivolumab and about 20,000 units hyaluronidase.

Embodiment 163. The method of any one of embodiments 157-159, wherein nivolumab co-formulated with hyaluronidase is administered to the subject about once every two to four weeks at a dose of 720 mg nivolumab and about 20,000 units hyaluronidase.

Embodiment 164. The method of any one of embodiments 157-159, wherein nivolumab co-formulated with hyaluronidase is administered to the subject about once every two to four weeks at a dose of 960 mg nivolumab and about 20,000 units hyaluronidase.

Embodiment 165. The method of any one of embodiments 157-159, wherein nivolumab co-formulated with hyaluronidase is administered to the subject about once every two to four weeks at a dose of 1200 mg nivolumab and about 20,000 units hyaluronidase.

Embodiment 166. The method of any one of embodiments 157-159, wherein nivolumab co-formulated with hyaluronidase is administered to the subject about once every two to four weeks at a dose of 720 mg nivolumab and about 12,000 units hyaluronidase.

Embodiment 167. The method of any one of embodiments 157-159, wherein nivolumab co-formulated with hyaluronidase is administered to the subject about once every two to four weeks at a dose of 960 mg nivolumab and about 16,000 units hyaluronidase.

Embodiment 168. The method of any one of embodiments 157-159, wherein the nivolumab co-formulated with hyaluronidase is administered to the subject about once every two to four weeks at a dose of 1200 mg nivolumab and about 20,000 units hyaluronidase.

Embodiment 169. The method of any one of embodiments 160-168, wherein the once every two to four weeks is once every two weeks.

Embodiment 170. The method of any one of embodiments 160-168, wherein the once every two to four weeks is once every three weeks.

Embodiment 171. The method of any one of embodiments 160-168, wherein the once every two to four weeks is once every four weeks.

Embodiment 172. The method of any one of embodiments 129-138, wherein the PD-1 therapy is pembrolizumab, and wherein pembrolizumab is administered to the subject about once every three weeks at a dose of 200 mg or about once every six weeks at a dose of 400 mg.

Embodiment 173. The method of any one of embodiments 129-138, wherein the PD-1 therapy is cemiplimab, and wherein cemiplimab is administered to the subject about once every three weeks at a dose of 350 mg.

Embodiment 174. The method of any one of embodiments 129-138, wherein the PD-1 therapy is dostarlimab, and wherein dostarlimab is administered to the subject about once every three weeks at a dose of 500 mg for dose 1 through dose 4 and about once every six weeks at a dose of 1000 mg for dose 5 onwards.

Embodiment 175. The method of any one of embodiments 129-138, wherein the PD-1 therapy is retifanlimab, and wherein retifanlimab is administered to the subject about once every four weeks at a dose of 500 mg.

Embodiment 176. The method of any one of embodiments 129-138, wherein the PD-1 therapy is toripalimab, and wherein toripalimab is administered to the subject about once every two weeks at a dose of 3 mg/kg.

Embodiment 177. The method of any one of embodiments 129-138, wherein the PD-1 therapy is atezolizumab, and wherein atezolizumab is administered to the subject about once every two weeks at a dose of 840 mg, once every three weeks at a dose of 1200 mg, or once every four weeks at a dose of 1680 mg.

Embodiment 178. The method of any one of embodiments 129-138, wherein the PD-1 therapy is avelumab, and wherein avelumab is administered to the subject about once every two weeks at a dose of 800 mg.

Embodiment 179. The method of any one of embodiments 129-138, wherein the PD-1 therapy is durvalumab, and wherein durvalumab is administered to the subject about once every two weeks at a dose of 10 mg/kg.

Embodiment 180. The method of any one of embodiments 104-179, wherein the method further comprises administering a chemotherapy.

Embodiment 181. The method of embodiment 180, wherein the chemotherapy comprises capecitabine and oxaliplatin administered in a cycling regimen of one or more 21-day cycles, wherein Day 1 is the first day of each cycle.

Embodiment 182. The method of embodiment 180 or embodiment 181, wherein capecitabine is administered to the subject about twice daily (BID) on Days 1 to 14 of each 21-day cycle at a dose of about 500 to about 1000 mg/m² and oxaliplatin is administered to the subject on Day 1 of each 21-day cycle at a dose of about 100 to about 150 mg/m².

Embodiment 183. The method of any one of embodiments 180-182, wherein capecitabine is administered to the subject about twice daily (BID) on Days 1 to 14 of each 21-day cycle at a dose of about 500 mg/m², about 750 mg/m², about 850 mg/m², or about 1000 mg/m² and oxaliplatin is administered to the subject on Day 1 of each 21-day cycle at a dose of about 130 mg/m².

Embodiment 184. The method of any one of embodiments 180-182, wherein capecitabine is administered to the subject about twice daily (BID) on Days 1 to 14 of each 21-day cycle at a dose of about 850 to about 1000 mg/m² and oxaliplatin is administered to the subject on Day 1 of each 21-day cycle at a dose of about 130 mg/m².

Embodiment 185. The method of any one of embodiments 180-184, wherein capecitabine is administered to the subject about twice daily (BID) on Days 1 to 14 of each 21-day cycle at a dose of about 1000 mg/m² and oxaliplatin is administered to the subject on Day 1 of each 21-day cycle at a dose of about 130 mg/m².

Embodiment 186. The method of embodiment 180, wherein the chemotherapy comprises oxaliplatin, folinic acid or a therapeutic equivalent thereof, and fluorouracil administered in a cycling regimen of one of more 28-day cycles, wherein Day 1 is the first day of each cycle.

Embodiment 187. The method of embodiment 186, wherein oxaliplatin is administered to the subject on Days 1 and 15 of each 28-day cycle at a dose of about 50 mg/m² to about 90 mg/m²; folinic acid or a therapeutic equivalent thereof is administered to the subject on Days 1 and 15 of each 28-day cycle at a dose of about 200 mg/m² to about 450 mg/m²; and fluorouracil is administered to the subject as a bolus on Days 1 and 15 of each 28-day cycle at a dose of about 200 mg/m² to about 450 mg/m² and as a continuous infusion starting on Days 1 and 15 of each 28-day cycle at a dose of about of about 1600 mg/m²/48 hours to about 2500 mg/m²/48 hours.

Embodiment 188. The method of embodiment 186 or embodiment 187, wherein oxaliplatin is administered to the subject on Days 1 and 15 of each 28-day cycle at a dose of about 50 mg/m², about 70 mg/m², or about 85 mg/m²; folinic acid or a therapeutic equivalent thereof is administered to the subject on Days 1 and 15 of each 28-day cycle at a dose of about 200 mg/m², about 300 mg/m², or about 400 mg/m²; and fluorouracil is administered to the subject as a bolus on Days 1 and 15 of each 28-day cycle at a dose of about 200 mg/m², about 300 mg/m², or about 400 mg/m² and as a continuous infusion starting on Days 1 and 15 of each 28-day cycle at a dose of about 1600 mg/m²/48 hours, about 2000 mg/m²/48 hours or about 2400 mg/m²/48 hours.

Embodiment 189. The method of any one of embodiments 186-188, wherein oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours.

Embodiment 190. The method of any one of embodiments 104-140, wherein the multispecific antibody is administered about once every two weeks at a dose of 30 mg in combination with nivolumab, wherein nivolumab is administered to the subject about once every two weeks at a dose of 240 mg.

Embodiment 191. The method of any one of embodiments 104-140, wherein the multispecific antibody is administered about once every two weeks at a dose of 30 mg in combination with nivolumab, wherein nivolumab is administered to the subject about once every three weeks at a dose of 360 mg.

Embodiment 192. The method of any one of embodiments 104-140, wherein the multispecific antibody is administered about once every two weeks at a dose of 30 mg in combination with nivolumab, wherein nivolumab is administered to the subject about once every four weeks at a dose of 480 mg.

Embodiment 193. The method of any one of embodiments 104-140, wherein the multispecific antibody is administered about once every two weeks at a dose of 90 mg in combination with nivolumab, wherein nivolumab is administered to the subject about once every two weeks at a dose of 240 mg.

Embodiment 194. The method of any one of embodiments 104-140, wherein the multispecific antibody is administered about once every two weeks at a dose of 90 mg in combination with nivolumab, wherein nivolumab is administered to the subject about once every three weeks at a dose of 360 mg.

Embodiment 195. The method of any one of embodiments 104-140, wherein the multispecific antibody is administered about once every two weeks at a dose of 90 mg in combination with nivolumab, wherein nivolumab is administered to the subject about once every four weeks at a dose of 480 mg.

Embodiment 196. The method of any one of embodiments 104-140, wherein the multispecific antibody is administered about once every two weeks at a dose of 250 mg in combination with nivolumab, wherein nivolumab is administered to the subject about once every two weeks at a dose of 240 mg.

Embodiment 197. The method of any one of embodiments 104-140, wherein the multispecific antibody is administered about once every two weeks at a dose of 250 mg in combination with nivolumab, wherein nivolumab is administered to the subject about once every three weeks at a dose of 360 mg.

Embodiment 198. The method of any one of embodiments 104-140, wherein the multispecific antibody is administered about once every two weeks at a dose of 250 mg in combination with nivolumab, wherein nivolumab is administered to the subject about once every four weeks at a dose of 480 mg.

Embodiment 199. The method of any one of embodiments 104-140, wherein the multispecific antibody is administered about once every two weeks at a dose of 500 mg in combination with nivolumab, wherein nivolumab is administered to the subject about once every two weeks at a dose of 240 mg.

Embodiment 200. The method of any one of embodiments 104-140, wherein the multispecific antibody is administered about once every two weeks at a dose of 500 mg in combination with nivolumab, wherein nivolumab is administered to the subject about once every three weeks at a dose of 360 mg.

Embodiment 201. The method of any one of embodiments 104-140, wherein the multispecific antibody is administered about once every two weeks at a dose of 500 mg in combination with nivolumab, wherein nivolumab is administered to the subject about once every four weeks at a dose of 480 mg.

Embodiment 202. The method of any one of embodiments 104-140, wherein the multispecific antibody is administered about once every two weeks at a dose of 1000 mg in combination with nivolumab, wherein nivolumab is administered to the subject about once every two weeks at a dose of 240 mg.

Embodiment 203. The method of any one of embodiments 104-140, wherein the multispecific antibody is administered about once every two weeks at a dose of 1000 mg in combination with nivolumab, wherein nivolumab is administered to the subject about once every three weeks at a dose of 360 mg.

Embodiment 204. The method of any one of embodiments 104-140, wherein the multispecific antibody is administered about once every two weeks at a dose of 1000 mg in combination with nivolumab, wherein nivolumab is administered to the subject about once every four weeks at a dose of 480 mg.

Embodiment 205. The method of any one of embodiments 104-140, wherein the multispecific antibody is administered in combination with nivolumab and a chemotherapy comprising capecitabine and oxaliplatin in a cycling regimen of one or more 21-day cycles, wherein Day 1 is the first day of each cycle, wherein the multispecific antibody is administered to the subject on Day 1 of each 21-day cycle at a dose of about 10 mg to about 1000 mg; wherein nivolumab is administered to the subject on Day 1 of each 21-day cycle at a dose of about 360 mg; wherein capecitabine is administered to the subject about twice daily (BID) on Days 1 to 14 of each 21-day cycle at a dose of about 1000 mg/m²; and oxaliplatin is administered to the subject on Day 1 of each 21-day cycle at a dose of about 130 mg/m².

Embodiment 206. The method of any one of embodiments 104-140, wherein the multispecific antibody is administered in combination with nivolumab and a chemotherapy comprising oxaliplatin, folinic acid or a therapeutic equivalent thereof, and fluorouracil in a cycling regimen of one or more 28-day cycles, wherein Day 1 is the first day of each cycle, wherein the multispecific antibody is administered to the subject on Days 1 and 15 of each 28-day cycle at a dose of about 10 mg to about 1000 mg; wherein nivolumab is administered to the subject on Days 1 and 15 of each 28-day cycle at a dose of about 240 mg; wherein oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours.

Embodiment 207. The method of embodiment 205 or embodiment 206, wherein the multispecific antibody is administered to the subject at a dose of about 10 mg.

Embodiment 208. The method of embodiment 205 or embodiment 206, wherein the multispecific antibody is administered to the subject at a dose of about 30 mg.

Embodiment 209. The method of embodiment 205 or embodiment 206, wherein the multispecific antibody is administered to the subject at a dose of about 90 mg.

Embodiment 210. The method of embodiment 205 or embodiment 206, wherein the multispecific antibody is administered to the subject at a dose of about 250 mg.

Embodiment 211. The method of embodiment 205 or embodiment 206, wherein the multispecific antibody is administered to the subject at a dose of about 500 mg.

Embodiment 212. The method of embodiment 205 or embodiment 206, wherein the multispecific antibody is administered to the subject at a dose of about 1000 mg.

Embodiment 213. The method of any one of embodiments 123-212, wherein each cycle of the cycling regimen is the same.

Embodiment 214. The method of any one of embodiments 104-213, wherein the cancer is NSCLC and the subject has not yet received treatment.

Embodiment 215. The method of any one of embodiments 104-213, wherein the cancer is NSCLC and the subject:
a) previously received platinum doublet-based chemotherapy and then progressed; or
b) was intolerant to platinum doublet-based chemotherapy.

Embodiment 216. The method of any one of embodiments 104-213 and 215, wherein the cancer is NSCLC and the subject:
a) previously received at least two prior lines of systemic therapy for advanced or metastatic disease and then progressed; or
b) was intolerant to at least two prior lines of systemic therapy for advanced or metastatic disease.

Embodiment 217. The method of any one of embodiments 104-213, 215, and 216, wherein the cancer is NSCLC and the subject has recurrent or progressive disease after completing platinum-based chemotherapy for local disease.

Embodiment 218. The method of any one of embodiments 104-213 and 215-217, wherein the cancer is NSCLC and the subject:
a) is administered the multispecific antibody in combination with nivolumab, and
b) has received previous treatment with a PD-1 therapy.

Embodiment 219. The method of any one of embodiments 104-213 and 215-218, wherein the cancer is NSCLC and the subject:
a) has one or more mutations in a protein selected from EGFR, ALK, ROS1, and RET, and
b) has received and progressed on, has been intolerant to, or was not a candidate for therapy with a tyrosine kinase inhibitor.

Embodiment 220. The method of any one of embodiments 104-213, wherein the cancer is SCCHN and the SCCHN is of the oral cavity, pharynx, or larynx.

Embodiment 221. The method of any one of embodiments 104-213 and 220, wherein the cancer is SCCHN of the oral cavity, pharynx, or larynx and the subject has not yet received treatment.

Embodiment 222. The method of any one of embodiments 104-213 and 220, wherein the cancer is SCCHN of the oral cavity, pharynx, or larynx and the subject:
a) previously received a platinum-containing regimen and then progressed, or
b) was intolerant to a platinum-containing regimen.

Embodiment 223. The method of any one of embodiments 104-213, 220, and 222, wherein the cancer is SCCHN of the oral cavity, pharynx, or larynx and the subject:
a) is administered the multispecific antibody in combination with nivolumab, and
b) has received previous treatment with a PD-1 therapy.

Embodiment 224. The method of any one of embodiments 104-213, wherein the cancer is PDAC and the subject has not yet received treatment.

Embodiment 225. The method of any one of embodiments 104-213, wherein the cancer is PDAC and the subject:
a) previously received at least one prior chemotherapy and then progressed; or
b) was intolerant to at least one prior chemotherapy.

Embodiment 226. The method of any one of embodiments 104-213, wherein the cancer is G/GEJC and the subject has not yet received treatment.

Embodiment 227. The method of any one of embodiments 104-213, wherein the cancer is G/GEJC and the subject:
a) previously received at least one prior standard treatment regimen in the advanced or metastatic setting and then progressed;
b) was intolerant to at least one prior standard treatment regimen in the advanced or metastatic setting; or
c) has progressed within 6 months of adjuvant therapy.

Embodiment 228. The method of any one of embodiments 104-213 and 227, wherein the cancer is G/GEJC and the subject:
a) is administered the multispecific antibody in combination with nivolumab, and
b) has received previous treatment with a PD-1 therapy.

Embodiment 229. The method of any one of embodiments 104-213, 227, and 228, wherein the G/GEJC is human epidermal growth factor receptor 2 (HER2)-positive G/GEJC, and wherein the subject has received prior treatment with a HER2 inhibitor.

Embodiment 230. The method of embodiment 229, wherein the HER2 inhibitor is trastuzumab.

Embodiment 231. The method of any one of embodiments 104-213, wherein the cancer is MSS CRC and the subject has not yet received treatment.

Embodiment 232. The method of any one of embodiments 104-213, wherein the cancer is MSS CRC and the subject:
a) previously received at least one standard systemic therapy for metastatic and/or unresectable disease and then progressed;
b) was intolerant to one standard systemic therapy for metastatic and/or unresectable disease; or
c) has progressed within 6 months of adjuvant therapy.

Embodiment 233. The method of any one of embodiments 104-213 and 232, wherein the cancer is MSS CRC and the subject has received prior treatment with fluoropyrimidine, oxaliplatin, and irinotecan given as a single regimen or over multiple regimens.

Embodiment 234. The method of any one of embodiments 104-213, 232, and 233, wherein the cancer is MSS CRC and the subject has proficient mismatch repair (MMR).

Embodiment 235. The method of any one of embodiments 104-213 and 232-234, wherein the cancer is MSS CRC and the subject has wild-type RAS and was previously treated with an anti-EGFR therapy.

Embodiment 236. The method of embodiment 235, wherein the anti-EGFR therapy is cetuximab or panitumumab.

Embodiment 237. The method of any one of embodiments 104-236, wherein treatment of the subject in need thereof is continued for about 1 month to about 24 months.

Embodiment 238. The method of any one of embodiments 104-237, wherein treatment of the subject in need thereof is continued for at least 1, 2, 3, 4, 5, or 6 months.

Embodiment 239. The method of any one of embodiments 104-238, wherein treatment of the subject in need thereof is continued until the subject achieves a complete response.

Embodiment 240. The method of embodiment 127, wherein the intravenous administration is completed over 30 minutes.

Embodiment 241. A multispecific antibody for use in treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Embodiment 242. A multispecific antibody for use in treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

Embodiment 243. A multispecific antibody for use in treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

Embodiment 244. A multispecific antibody for use in treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

Embodiment 245. Use of a multispecific antibody in the manufacture of a medicament for treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Embodiment 246. Use of a multispecific antibody in the manufacture of a medicament for treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

Embodiment 247. Use of a multispecific antibody in the manufacture of a medicament for treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

Embodiment 248. Use of a multispecific antibody in the manufacture of a medicament for treating a subject according to the method of any one of embodiments 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

It is to be understood that both the foregoing general description and the following detailed description are exemplary and explanatory only and are not restrictive of the claims. All references cited herein are incorporated in their entirety by reference.

### BRIEF DESCRIPTION OF THE DRAWINGS

FIG. 1 shows binding of the CD40.9H3×FAP.LP62 bispecific antibody (CD40.9H3×FAP.LP62) to various cell types from human blood. Each data point represents a single donor for each cell population (n=4 donors shown). The percentage of each cell type to which CD40.9H3×FAP.LP62 bound was quantified by flow cytometry.

FIGS. 2A-2C show antibody-dependent cellular cytotoxicity (ADCC) (FIG. 2A), complement-dependent cytotoxicity (CDC) (FIG. 2B), and antibody-dependent cellular phagocytosis (ADCP) (FIG. 2C) activity of CD40.9H3×FAP.LP62 on CD40-positive Raji cells in the presence of human NK cells, complement, and macrophages from healthy human donors (representative data from n=3 donors across 3 independent experiments, n=3 independent experiments, and n=2 donors across 2 independent experiments, respectively). Anti-CD20 antibody was included as a positive control and isotype control antibody was used as a negative control.

FIGS. 3A-3B show activation of human dendritic cells (DCS) (FIG. 3A) and macrophages (FIG. 3B). FIG. 3A shows IL12p40 secretion in response to increasing amounts of CD40.9H3×FAP.LP62 in a co-culture assay of DCs with HFF1 or HFF1 FAP knockout (KO) cells (representative data from n=8 donors across 4 independent experiments). FIG. 3B shows TNFα secretion in response to increasing amounts of CD40.9H3×FAP.LP62 in a co-culture assay of macrophages with HFF1 or HFF1 FAP KO cells (representative data from n=6 donors across 3 independent experiments).

FIGS. 4A-4B show CCL22 (FIG. 4A) and IL12p40 (FIG. 4B) secretion in response to increasing amounts of mCD40×FAP.LP62 in a co-culture assay of murine bone marrow-derived macrophages (BMDMs) with MC38-FAP or MC38-FAP KO cells (representative data from 4 pooled mice across 2 independent experiments).

FIG. 5 shows cumulative KPCY tumor growth curves upon treatment (QDx1) with 3 or 10 mg/kg mCD40×FAP.LP62 compared to mCD40×HEL (hen egg lysozyme) isotype control. Data shown are representative of 10 mice per group across 2 independent experiments. Statistical significance was determined by 2-way Anova (repeat measure). **P<0.01, ***P<0.001; ****P<0.0001.

FIGS. 6A-6E shows immune profiling of KPCY tumor-bearing mice dosed with 10 mg/kg mCD40×FAP.LP62 or isotype control. Tumor cross-presenting dendritic cell (cDC1) CD86 expression (FIG. 6A), % migratory cDC1 cells (FIG. 6B), and CD86 expression of cDC1 cells that migrated to tumor-draining lymph nodes (TDLN) (FIG. 6C) are all increased at 48 hours. Resident cDC1 cells in TDLNs showed only modest (p<0.01), if any, increase, in CD86 activation (FIG. 6D). The ratio of CD8+ T cells to Tregs (CD8:Treg) in tumors was increased at 240 hours (FIG. 6E). Error bars indicate mean±SD. Statistical analyses were performed by using unpaired parametric Student's t-test with Welch's correction. *P<0.05, **P<0.01, ***P<0.001, ****P<0.0001. Data shown are representative of 10 mice per group across 2 independent experiments.

FIG. 7 shows cytokine levels in KPCY tumors 240 hours post-treatment with mCD40×FAP.LP62 or mCD40×HEL isotype control (10 mg/kg, QDx1). Statistical significance was determined by Student's T test (nonparametric, Mann-Whitney). **P<0.01, ****P<0.0001. Data are representative of 10 mice per group across 2 independent experiments for the isotype control and 3 and 10 mice per group for mCD40×FAP.LP62 across two independent experiments.

FIG. 8A shows MC38-FAP tumor volumes upon treatment (QDx1) with 8.6 mg/kg anti-CD40, equimolar 10 mg/kg mCD40×FAP.LP62, or 30 mg/kg mCD40×HEL (hen egg lysozyme) isotype control. Data shown represent 10 mice per group within 1 experiment. Statistical significance was determined by 2-way Anova (repeat measure). **P<0.01, ***P<0.001; ****P<0.0001. FIG. 8B and FIG. 8C show alanine aminotransferase (ALT; serum, 4-5 mice per group) and cytokine (scrum, 5 mice per group; tumor 2-5 mice per group) levels in MC38-FAP tumor-bearing mice at 24 hours post-treatment (QDx1) with 10.3 mg/kg anti-CD40, equimolar 12 mg/kg mCD40×FAP.LP62, or 12 mg/kg mCD40×HEL (hen egg lysozyme) isotype control. Data shown represent 1 experiment.

FIG. 9 shows cytokine release (CCL22, MIP-1B, CCL17, MIP-1α, IL6, IL12p40) from dissociated human patient tumors treated with 5 nM (unless otherwise noted) CD40.9H3×FAP.LP62, an analog of RO7300490, or an anti-HEL×FAP isotype control antibody for 24 hours. Data shown represent 5 different tumor samples: 2 pancreatic, 2 non-small cell lung (1 treated at 0.05 nM), and 1 squamous lung.

FIG. 10A shows internalization of CD40.9H3×FAP.LP62 and an analog of RO7300490 upon binding to FAP expressed on HFF1 cells compared to HFF1 FAP KO control cell lines. FIG. 10B shows internalization of CD40.9H3×FAP.LP62 and the analog of RO7300490 upon binding to CD40 expressed on Raji cells as compared to anti-HEL×FAP isotype control antibody. Data shown represent 2 independent experiments.

FIG. 11 shows IL12p40 cytokine release from human dendritic cells in response to increasing amounts of CD40.9H3×FAP.LP62 or an analog of RO7300490 in the presence of absence of 120 ng/mL and 480 ng/mL soluble FAP in a co-culture assay of DCs with HFF1 or HFF1 FAP KO cells (representative data from n=2 donors within 1 experiment).

FIG. 12 shows IL2 cytokine release from CD4 T cells co-cultured with macrophages and HFF1 cells in response to increasing amounts of CD40.9H3×FAP.LP62 or an analog of MP0317. Anti-HEL×FAP was included as an isotype control (representative data from n=3 donors across 2 independent experiments).

FIG. 13 shows combination effect of CD40.9H3×FAP.LP62 and anti-PD1 antibody to enhance IFNγ secretion in tri-culture assay of DCs, HFF1 cells and T cells (n=3 DC and T cell donor pairs across 2 independent experiments).

FIG. 14 shows cumulative KPCY tumor growth curves upon treatment with mCD40×FAP.LP62 (3 mg/kg, QDx1) and/or anti mPD1 antibody (10 mg/kg, QW×3) compared to mCD40×HEL (10 mg/kg, QDx1)+anti-KLH (10 mg/kg, QW×3) isotype control antibodies. Data shown are representative of 10 mice per group across 2 independent experiments. Statistical significance was determined by 2-way Anova (repeat measure). **P<0.01, ***P<0.001; ****P<0.0001.

FIG. 15 shows the study design for the Phase 1/1b clinical trial of CD40.9H3×FAP.LP62 as a monotherapy or in combination with an anti-PD-1 antibody nivolumab for the treatment of NSCLC, PDAC, MSS CRC, SCCHN, and G/GEJC. The study includes a Part I escalation study and a Part II expansion study. Abbreviations: DL, dose level; G/GEJC, gastric/gastroesophageal junction adenocarcinoma; IV, intravenous; MAD, maximum administered dose; MSS CRC, microsatellite stable colorectal cancer; MTD, maximum tolerated dose; NSCLC, non-small cell lung cancer; PDAC, pancreatic ductal adenocarcinoma; Q2W/Q4W, every 2/4 weeks; SCCHN, squamous cell carcinoma of the head and neck; TBD, to be determined.

FIG. 16 shows the study design for the Phase 1/1b clinical trial of CD40.9H3×FAP.LP62 as a monotherapy or in combination with an anti-PD-1 antibody nivolumab and/or chemotherapy for the treatment of NSCLC, PDAC, MSS CRC, SCCHN, and G/GEJC. The study includes a Part I escalation study and a Part II expansion study. Abbreviations: DL, dose level; G/GEJC, gastric/gastroesophageal junction adenocarcinoma; IV, intravenous; MSS CRC, microsatellite stable colorectal cancer; NSCLC, non-small cell lung cancer; PDAC, pancreatic ductal adenocarcinoma; Q2W/Q3W/Q4W, every 2/3/4 weeks; SC, subcutaneous; SCCHN, squamous cell carcinoma of the head and neck; TBD, to be determined.

FIG. 17 shows an illustration of a CD40.9H3×FAP.LP62 bispecific antibody (CD40.9H3×FAP.LP62).

### DETAILED DESCRIPTION OF CERTAIN EMBODIMENTS

Targeting the CD40 pathway has shown promise in eliciting clinical responses in patients with advanced solid tumors. Activation of the costimulatory receptor, CD40, on dendritic cells (DCs) and macrophages drives transcriptional programs that remodel and inflame the tumor microenvironment as well as enhance T-cell priming. Because CD40 serves such an important role in both innate and adaptive immunity, targeting this pathway holds the potential for improved and durable patient benefit both as monotherapy, as well as in combination with standard of care therapies, including chemotherapy and T-cell checkpoint inhibitors. However, approaches thus far have largely activated CD40 systemically, resulting in a limited therapeutic window. In some embodiments, CD40.9H3×FAP.LP62 potently agonizes the CD40 pathway selectively within the fibroblast activation protein (FAP)-enriched tumor stroma of multiple solid tumor indications, including but not limited to, pancreatic, gastric, lung, colorectal, and others, thereby augmenting the anti-tumor immune response locally to drive clinical benefit in patients with advanced solid malignancies, while concurrently ameliorating systemic toxicities.

### I. DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et al. Molecular Cloning: A Laboratory Manual (2nd ed., Cold Spring Harbor Laboratory Press, Cold Spring Harbor, N.Y. (1989)), among other places.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term about may include numerical values that are rounded to the nearest significant figure.

The term “polypeptide” refers to a polymer of amino acid residues, and is not limited to a minimum length. A “protein” may comprise one or more polypeptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” or “protein” refers to a polypeptide or protein, respectively, which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts that produce the proteins or errors due to PCR amplification. A protein may comprise two or more polypeptides.

“CD40” or “cluster of differentiation 40” or “tumor necrosis factor receptor superfamily member 5” or “TNFRSF5” as used herein, refers to human CD40 (UniProt ID: P25942.1; NP_001241.1), unless expressly noted otherwise (i.e., murine CD40, cynomolgus CD40, or the like). An exemplary mature human CD40 amino acid sequence is shown in SEQ ID NO: 36.

“FAP” or “fibroblast activation protein alpha” as used herein, refers to human FAP (UniProt ID: Q12884.1; NP_004451.2), unless expressly noted otherwise (i.e., murine FAP, cynomolgus FAP, or the like). An exemplary human FAP amino acid sequences is shown in SEQ ID NO: 37. FAP is expressed in some tumor tissues, for example, in the tumor microenvironment (TME), including during development of malignant tumors. For example, cancer-associated fibroblasts (CAFs) in the tumor stroma can have high levels of FAP expression, where it plays a role in promoting tumor growth, invasion, metastasis, and immunosuppression.

The term “antibody” herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired target-specific binding activity.

An “isolated” antibody is one that has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antigen” refers to the target of an antibody, i.e., the molecule to which the antibody specifically binds. The term “epitope” denotes the site on an antigen, either proteinaceous or non-proteinaccous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e., by the tertiary folding of a proteinaccous antigen. Linear epitopes are typically still bound by an antibody after exposure of the proteinaceous antigen to denaturing agents, whereas conformational epitopes are typically destroyed upon treatment with denaturing agents.

An “anti-CD40 antibody” or a “CD40-antibody” or an “antibody that specifically binds to CD40” or an “antibody that binds to CD40” and similar phrases refer to an antibody that specifically binds to CD40.

An “anti-FAP antibody” or a “FAP-antibody” or an “antibody that specifically binds to FAP” or an “antibody that binds to FAP” and similar phrases refer to an antibody that specifically binds to FAP.

A “multispecific anti-CD40/FAP antibody” or a “bispecific anti-CD40/FAP antibody” or a “multivalent bispecific anti-CD40/FAP antibody” or a “CD40×FAP-antibody” or an “anti-CD40 anti-FAP-antibody” or an “antibody that specifically binds to CD40 and FAP” or an “antibody that binds to CD40 and FAP” and similar phrases refer to an antibody that comprises at least one antigen binding domain that specifically binds to CD40 and at least one antigen binding domain that specifically binds to FAP. A multispecific or bispecific anti-CD40/FAP antibody may comprise more than one antigen binding domain that binds to CD40, or more than one antigen binding domain that binds to FAP, so long as the antibody comprises at least one antigen binding domain that specifically binds to CD40 and at least one antigen binding domain that specifically binds to FAP. The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “complementarity determining regions” (“CDRs”) as used herein refers to each of the regions of an antibody variable region which are hypervariable in sequence and which determine antigen binding specificity. Generally, antibodies comprise six CDRs: three in the VH (CDR-H1 or heavy chain CDR1, CDR-H2, CDR-H3), and three in the VL (CDR-L1, CDR-L2, CDR-L3). Unless otherwise indicated, the CDRs are determined according to the sequence table herein. In some embodiments, CDRs are determined according to Kabat definitions. In some embodiments, CDRs are determined according to IMGT.

“Framework” or “framework region” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). Sec, e.g., Kindt et al. Kuby Immunology, 6^{th} ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. Sec, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, MD, 1991.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell, NK cell, and macrophage activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” or “antigen-binding fragment” or “antigen-binding portion” refers to a fragment or portion of an antibody other than an intact antibody that binds the antigen (e.g., CD40 or FAP) to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein above.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” as used herein refers to antibodies produced from human immunoglobulin sequences, such as antibodies produced in non-human animals that comprise human immunoglobulin genes (such as XenoMouse® and VelocImmune® micc), and antibodies selected using in vitro methods, such as phage display, wherein the antibody repertoire is based on a human immunoglobulin sequence.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method.

A “multispecific” antibody is one that binds specifically to more than one target antigen, while a “bispecific” antibody is one that binds specifically to two antigens. An “antibody conjugate” is an antibody conjugated to one or more heterologous molecule(s), including but not limited to a therapeutic agent or a label.

Antibodies may be modified as part of the production process in certain host cells or through metabolism in vivo. An antibody or antibody region amino acid sequence herein is intended to encompass not only the specific amino acid sequence, but also that sequence as post-translationally modified, for instance, including side chain modifications and cleavages. Such a post-translational modification can occur, for instance, as a result of production of the antibody in a host cell and/or as a result of post-translational modification in vivo in an animal (e.g., a human).

In some embodiments, an antibody disclosed herein comprises a post-translational modification (e.g., one or more post-translational modifications). Post-translational modifications can include, e.g., ubiquitination, phosphorylation, acetylation, hydroxylation, methylation, glycosylation, AMPylation, prenylation, deamidation, citrullination, and carbamoylation. In some embodiments, the antibody is not post-translationally modified.

As noted above, antibodies can undergo post-translational cleavage of one or more, particularly one or two, amino acids from the C-terminus of the heavy chain, often a Gly-Lys. This cleavage can occur, for instance, as a result of the process of production of the antibody in a host cell. An antibody produced by expression of a specific nucleic acid molecule encoding a full-length heavy chain can include the full-length heavy chain, or it can include a cleaved variant of the full-length heavy chain, such as a heavy chain lacking a C-terminal Lys or a C-terminal Gly-Lys.

Other types of post-translational modifications can occur during production of antibodies, or otherwise in vivo, such as the modification of an amino acid side chain. For instance, an N-terminal Glu or Gln residue on an antibody chain can be post-translationally modified to an N-terminal pyroglutamate (also known as pyrrolidine carboxylate; abbreviated pE).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “signal sequence” or “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Nonlimiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The term “nucleic acid molecule” or “polynucleotide” includes any compound and/or substance that comprises a polymer of nucleotides. Each nucleotide is composed of a base, specifically a purine- or pyrimidine base (i.e., cytosine I, guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA, circular RNA) vectors, can be unmodified or modified.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD40 antibody” refers to one or more nucleic acid molecules encoding anti-CD40 antibody heavy and light chains (or fragments thereof), including such nucleic acid molecule(s) in a single vector or separate vectors, and such nucleic acid molecule(s) present at one or more locations in a host cell.

“Isolated nucleic acid encoding an anti-FAP antibody” refers to one or more nucleic acid molecules encoding anti-FAP antibody heavy and light chains (or fragments thereof), including such nucleic acid molecule(s) in a single vector or separate vectors, and such nucleic acid molecule(s) present at one or more locations in a host cell.

“Isolated nucleic acid encoding a multispecific [or bispecific] anti-CD40/FAP antibody” refers to one or more nucleic acid molecules encoding anti-CD40 and/or anti-FAP antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells.

In this disclosure, “binds” or “binding” or “specific binding” and similar terms, when referring to a protein and its ligand or an antibody and its antigen target for example, or some other binding pair, means that the binding affinity between the members of the binding pair is sufficiently strong that the interaction cannot be due to random molecular associations (i.e. “nonspecific binding”). Such binding typically requires a dissociation constant (K_{D}) of 1 μM or less, and may often involve a K_{D} of 100 nM or less.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). Affinity can generally be represented by the dissociation constant (K_{D}). Affinity of an antibody for an antigen can be measured by common methods known in the art, such as biolayer interferometry or surface plasmon resonance (SPR), for instance. Unless otherwise indicated, K_{D} values provided herein were determined using biolayer interferometry.

The terms “reduce” or “inhibit” more generally refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Treatment” or “treating” as used herein, covers any administration or application of a therapeutic for disease in a human, and includes inhibiting the disease or progression of the disease or one or more disease symptoms, inhibiting or slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless expressly indicated otherwise (i.e., a murine subject or the like).

A “PD-1 therapy” as used herein encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapy. PD-1 therapies include, but are not limited to, nivolumab, pembrolizumab, cemiplimab, dostarlimab, retifanlimab, toripalimab, atezolizumab, avelumab, and durvalumab.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells.

As used herein, “solid cancer” or “solid tumor” refers to abnormally high levels of proliferation and growth in one or more solid organ and/or tissue, including but not limited to the stomach, colon, pancreas, lungs, breast, head and neck, and mesothelium. The solid tumor has an associated “tumor microenvironment” (or “TME”), which may comprise, for example, cells (e.g., immune cells and stromal cells), molecules and metabolites, blood vessels, and an extracellular matrix (ECM) that surrounds the solid tumor and supports cancer cell survival, local invasion, and/or metastatic dissemination. In some instances, an antibody binds an antigen expressed in the tumor microenvironment. In some instances, an antibody binds FAP expressed in the tumor microenvironment.

Nonlimiting exemplary solid cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

As used herein, the term “tumor,” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The term “microsatellite instability” or “MSI” as used herein refers to a change occurring in certain cells, e.g., cancer cells, in which the number of repeated DNA bases in a microsatellite (i.e., a short, repeated sequence of DNA) is different from what it was when the microsatellite was inherited. MSI status can be determined by a molecular test, for example, polymerase chain reaction (PCR) and immunohistochemistry (IHC). In some embodiments, MSI testing by PCR is considered the standard method for MSI detection, wherein common microsatellite markers are examined and quantified. In some embodiments, MSI detection includes analysis of 5 markers (loci): BAT-25, BAT-26, NR21, NR24 and MONO-27/NR27. In some embodiments, genetic instability (e.g., an expansion or reduction in the length of the microsatellites) in 2 or more of the 5 markers indicates MSI-high cancer. This list of microsatellite markers is not exclusive, as other markers can be used for the purposes of determining MSI status (e.g., dinucleotide repeats D2S123, D5S346, and D17S250 of the Bethesda panel). MSI status can be high (MSI-H), low (MSI-L), or stable (MSS). MSI-L tumor is characterized by genetic instability in 1 of the 5 tested markers (loci). MSS refers to a tumor comprising genetic instability (e.g., an expansion or reduction in the length of the microsatellites) in none of the 5 markers (loci).

The terms “microsatellite instability positive” and “MSI-positive” refer to tumors that are MSI-high or MSI-low. A tumor is also considered to be MSI-positive if one or more mismatch repair (MMR) proteins selected from MLH1, MSH2, PMS2, and MSH6 are absent by immunohistochemistry (IHC).

The term “mismatch repair deficiency,” “MMR deficiency,” “deficient MMR,” or “dMMR” as used herein refers to tumors having one or more mutations in one or more DNA mismatch repair genes. MMR status (i.e., deficiency or proficiency) can be determined by a molecular test, for example, immunohistochemistry (IHC), genetic testing by sequencing, and/or deletion-duplication analysis. In some embodiments, IHC for loss of expression of one or more MMR proteins (e.g., MLH1, MSH2, MSH6, and PMS2) may be used. This list of MMR proteins is not exclusive, as other MMR proteins can be used for the purposes of determining MMR status.

The term “proficient MMR,” “pMMR,” or “MMR proficiency” as used herein refers to tumors that are microsatellite stable (MSS) and MSI-low (MSI-L) or tumors with intact MMR protein expression.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

A “biological sample” as used herein refers to a sample taken from a subject or from an animal. Examples of biological samples include tissue samples and liquid biological samples, such as whole blood, serum, plasma, blood supernatant, or synovial fluid. A biological sample may be taken directly from a subject or may be first chemically or physically modified in some fashion prior to use, for example, in order to assist in analysis of the sample.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### II. EXEMPLARY ANTI-CD40 ANTIBODIES

In some embodiments, antibodies comprising at least one antigen binding domain that specifically binds CD40, such as human CD40, are provided. Such antibodies include, but are not limited to, monoclonal antibodies, multispecific antibodies, humanized antibodies, chimeric antibodies, mouse antibodies, human antibodies, and antibodies comprising the heavy chain and/or light chain CDRs discussed herein. In some embodiments, an isolated antibody comprising at least one antigen binding domain that binds to CD40 is provided. In some embodiments, a monoclonal antibody comprising at least one antigen binding domain that binds to CD40 is provided. In some embodiments, the antibody binds to human CD40. In some embodiments, the antibody binds to human CD40 comprising the amino acid sequence of SEQ ID NO: 36.

In some embodiments, an antigen binding domain that binds CD40 comprises a heavy chain variable region and a light chain variable region. In some embodiments, an antibody comprising at least one antigen binding domain that binds CD40 comprises at least one heavy chain comprising a heavy chain variable region and at least a portion of a heavy chain constant region, and at least one light chain comprising a light chain variable region and at least a portion of a light chain constant region. In some embodiments, an antibody comprising at least one antigen binding domain that binds CD40 comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain variable region and a light chain variable region. In some embodiments, the heavy chain variable region is the region of the anti-CD40 antibody that comprises the three heavy chain CDRs. In some embodiments, the light chain variable region is the region of the anti-CD40 antibody that comprises the three light chain CDRs.

In some embodiments, an antigen binding domain that binds CD40 comprises at least one, two, three, four, five, or six CDRs selected from a heavy chain complementarity determining region 1 (HCDR1) comprising an amino acid sequence selected from SEQ ID NO: 1 or 7; a heavy chain complementarity determining region 2 (HCDR2) comprising an amino acid sequence selected from SEQ ID NO: 2 or 8; a heavy chain complementarity determining region 3 (HCDR3) comprising the amino acid sequence of SEQ ID NO: 3 or 9; a light chain complementarity determining region 1 (LCDR1) comprising an amino acid sequence selected from SEQ ID NO: 4 or 10; a light chain complementarity determining region 2 (LCDR2) comprising an amino acid sequence selected from SEQ ID NO: 5 or 11; and a light chain complementarity determining region 3 (LCDR3) comprising the amino acid sequence of SEQ ID NO: 6 or 12.

In some embodiments, the antigen binding domain that binds CD40 comprises six CDRs including a HCDR1 comprising an amino acid sequence selected from SEQ ID NO: 1 or 7; a HCDR2 comprising an amino acid sequence selected from SEQ ID NO: 2 or 8; a HCDR3 comprising the amino acid sequence of SEQ ID NO: 3 or 9; a LCDR1 comprising an amino acid sequence selected from SEQ ID NO: 4 or 10; a LCDR2 comprising an amino acid sequence selected from SEQ ID NO: 5 or 11; and a LCDR3 comprising the amino acid sequence of SEQ ID NO: 6 or 12.

In some embodiments, an antigen binding domain that binds CD40 comprises at least one, two, three, four, five, or six CDRs selected from a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; a LCDR1 comprising an amino acid sequence selected from SEQ ID NO: 4; a LCDR2 comprising an amino acid sequence selected from SEQ ID NO: 5; and a LCDR3 comprising the amino acid sequence of SEQ ID NO: 6.

In some embodiments, an antigen binding domain that binds CD40 comprises at least one, two, three, four, five, or six CDRs selected from a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; a LCDR1 comprising an amino acid sequence selected from SEQ ID NO: 10; a LCDR2 comprising an amino acid sequence selected from SEQ ID NO: 11; and a LCDR3 comprising the amino acid sequence of SEQ ID NO: 12.

In some embodiments, the antigen binding domain that binds CD40 comprises six CDRs including a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; a LCDR1 comprising an amino acid sequence selected from SEQ ID NO: 4; a LCDR2 comprising an amino acid sequence selected from SEQ ID NO: 5; and a LCDR3 comprising the amino acid sequence of SEQ ID NO: 6.

In some embodiments, the antigen binding domain that binds CD40 comprises six CDRs including a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; a LCDR1 comprising an amino acid sequence selected from SEQ ID NO: 10; a LCDR2 comprising an amino acid sequence selected from SEQ ID NO: 11; and a LCDR3 comprising the amino acid sequence of SEQ ID NO: 12.

In some embodiments, any of the six anti-CD40 CDRs as defined by Kabat can be combined as subparts with any of the anti-CD40 CDRs as defined by IMGT, for a total of six CDRs in a construct. Thus, in some embodiments, two CDRs defined by Kabat (for example, HCDR1 and HCDR2) can be combined with four CDRs as defined by IMGT (HCDR3, LCDR1, LCDR2, and LCDR3). In some embodiments, two or fewer residues in one or more of the CDRs can be replaced to obtain a variant thereof. In some embodiments, two or fewer residues can be replaced in 1, 2, 3, 4, 5, or 6 of the CDRs.

In some embodiments, the antibody comprising at least one antigen binding domain that binds CD40 comprises (I) a heavy chain variable region (VH) comprising at least one, at least two, or all three VH CDR sequences selected from (i) an HCDR1 comprising an amino acid sequence selected from SEQ ID NO: 1 or 7; (ii) an HCDR2 comprising an amino acid sequence selected from SEQ ID NO: 2 or 8; (iii) an HCDR3 comprising the amino acid sequence of SEQ ID NO: 3 or 9; and (II) a VL domain comprising at least one, at least two, or all three VL CDR sequences selected from (iv) an LCDR1 comprising an amino acid sequence selected from SEQ ID NO: 4 or 10; (v) an LCDR2 comprising an amino acid sequence selected from SEQ ID NO: 5 or 11; and (vi) an LCDR3 comprising the amino acid sequence of SEQ ID NO: 6 or 12; wherein the VH comprises a sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 13, and the VL domain comprises a sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 14.

In some embodiments, the antibody comprising at least one antigen binding domain that binds CD40 comprises a heavy chain variable region (VH) sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 13. In some embodiments, a VH sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but the antigen binding domain comprising that sequence retains the ability to bind CD40. In some embodiments, a total of 1 to 10 amino acids have been substituted, inserted and/or deleted in SEQ ID NO: 13. In some embodiments, substitutions, insertions, or deletions occur in regions outside the CDRs (that is, in the framework regions (FRs)). In some embodiments, the antigen binding domain that binds CD40 comprises the VH sequence of SEQ ID NO: 13. In some embodiments, the antigen binding domain that binds CD40 comprises the VH sequence of SEQ ID NO: 13, including post-translational modifications of that sequence.

In some embodiments, an antibody comprising at least one antigen binding domain that binds CD40 is provided, wherein the antibody comprises a light chain variable region (VL) having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 14. In some embodiments, a VL sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain comprising that sequence retains the ability to bind to CD40. In some embodiments, a total of 1 to 10 amino acids have been substituted, inserted and/or deleted in SEQ ID NO:14. In some embodiments, the substitutions, insertions, or deletions occur in regions outside the CDRs (that is, in the FRs). In some embodiments, the antigen binding domain that binds CD40 comprises the VL sequence of SEQ ID NO: 14. In some embodiments, the antigen binding domain that binds CD40 comprises the VL sequence of SEQ ID NO: 14, including post-translational modifications of that sequence.

In some embodiments, an antibody comprising at least one antigen binding domain that binds CD40 comprises a heavy chain variable region (VH) sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 13 and a light chain variable domain (VL) having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 14. In some embodiments, a VH sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, and a VL sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain comprising that sequence retains the ability to bind to CD40. In some embodiments, a total of 1 to 10 amino acids have been substituted, inserted and/or deleted in SEQ ID NO: 13. In some embodiments, a total of 1 to 10 amino acids have been substituted, inserted and/or deleted in SEQ ID NO: 14. In some embodiments, substitutions, insertions, or deletions occur in regions outside the CDRs (that is, in the FRs). In some embodiments, the antigen binding domain that binds CD40 comprises the VH sequence of SEQ ID NO: 13 and the VL sequence of SEQ ID NO: 14. In some embodiments, the antigen binding domain that binds CD40 comprises the VH sequence of SEQ ID NO: 13 and the VL sequence of SEQ ID NO: 14, including post-translational modifications of one or both sequences.

In various embodiments, the antibody is a monoclonal antibody. In various embodiments, the antibody is a multispecific antibody.

In various embodiments, an antibody comprising at least one antigen binding domain that binds CD40 may comprise any of the following properties, singly or in combination. In some embodiments, the antibody binds to human CD40 with an affinity (K_{D}) between 20 and 200 nM, or 50 and 200 nM, or 50 and 150 nM, as determined by surface plasmon resonance. In some embodiments, the antibody binds to cynomolgus monkey CD40 with a K_{D} between 20 and 200 nM, or 50 and 200 nM, or 50 and 150 nM, as determined by surface plasmon resonance. In some embodiments, the antibody binds human CD40 expressed on the surface of cells with an EC50 of 1-50 nM, or 1-25 nM, or 3-20 nM, or 3-15 nM.

In some embodiments, the antibody binds human CD40. In some embodiments, the human CD40 comprises the amino acid sequence of SEQ ID NO: 36.

### III. EXEMPLARY MULTISPECIFIC ANTI-CD40/FAP ANTIBODIES

In certain embodiments, an antibody provided herein is a multispecific antibody, for example, a bispecific antibody. Multispecific antibodies are monoclonal antibodies that have binding specificities for at least two different target antigens. In certain embodiments, at least one antigen binding domain of a multispecific antibody binds to CD40 and at least one antigen binding domain of a multispecific antibody binds to FAP. In some embodiments, a multispecific antibody is a trivalent bispecific antibody. A trivalent bispecific antibody comprises three antigen binding domains, wherein two antigen binding domains bind to a first antigen, such as CD40, and one antigen binding domain binds to a second antigen, such as FAP. Multispecific antibodies can be prepared as full-length antibodies or antibody fragments, or may comprise a full-length antibody fused to an antibody fragment. In some embodiments, a multispecific antibody comprises a full-length antibody and a scFv, wherein the scFv is fused to one heavy chain constant region of the full-length antibody.

In some embodiments, a multispecific antibody provided herein is a bispecific antibody comprising a first antigen binding domain that binds CD40 and a second antigen binding domain that binds a second antigen. In some embodiments, the second antigen is overexpressed in cancer cells. In some embodiments, the second antigen is highly expressed in the tumor microenvironment. In some embodiments, the second antigen is FAP. In various embodiments, the first antigen binding domain is an antigen binding domain that binds CD40 described herein. In some embodiments, the multispecific antibody is an immune cell engager. In some embodiments, the first antigen binding domain binds an antigen on the surface of, for example, dendritic cells (DCs), macrophages, and/or B cells. In some embodiments, the second antigen binding domain binds an antigen on the surface of cancer cells in the tumor microenvironment. In some embodiments, the second antigen binding domain of the bispecific antibody binds FAP. In some such embodiments, the multispecific antibody is a CD40 agonist in the presence of FAP-expressing cells. In some embodiments, the multispecific antibody does not compete with CD40L for binding to CD40.

In some embodiments, a multispecific antibody is provided, wherein the multispecific antibody comprises a first antigen binding domain that binds CD40 and a second antigen binding domain that binds CD40. In some embodiments, the multispecific antibody comprises a third antigen binding domain that binds FAP, wherein the first and second antigen binding domains are the same or different. In some embodiments, the third antigen binding domain that binds FAP is a scFv.

Also provided herein is a multispecific antibody comprising a first antigen binding domain that binds CD40, a second antigen binding domain that binds CD40, and a third antigen binding domain that binds fibroblast activation protein alpha (FAP), wherein the third antigen binding domain is a single-chain variable region (scFv). In some embodiments, the multispecific antibody comprises a full-length antibody comprising a first antigen binding domain that binds CD40 and a second antigen binding domain that binds CD40. In some embodiments, the multispecific antibody further comprises an scFv fused to the C-terminus of a first heavy chain constant region of the full-length antibody.

In some embodiments, the multispecific antibody is a 2+1 bispecific antibody. In some embodiments, the 2+1 bispecific antibody comprises a first antigen binding domain that binds CD40, a second antigen binding domain that binds CD40, and a third antigen binding domain that binds fibroblast activation protein alpha (FAP). In some embodiments, the 2+1 format provides a trivalent bispecific antibody that includes three antigen binding sites, namely two antigen binding sites for CD40 and one antigen binding site for FAP. In some embodiments, each antigen binding domain that binds to CD40 is monovalent and together provide for bivalent binding to CD40. In some embodiments, the antigen binding domain that binds to CD40 is a Fab antigen binding fragment. In some embodiments, the third antigen binding domain is a single-chain variable region (scFv). In some embodiments, the provided multispecific antibody is bivalent binding to CD40 and monovalent for binding to FAP.

A multispecific antibody comprising at least one antigen binding domain that binds CD40 may comprise any of the antigen binding domains that bind CD40 provided herein. In some embodiments, a multispecific antibody comprises two antigen binding domains that bind CD40, which may be the same or different. In some embodiments, a multispecific antibody comprises two antigen binding domains that bind CD40, which are the same.

A multispecific antibody comprising at least one antigen binding domain that binds CD40 and at least one binding domain that binds FAP may comprise any of the antigen binding domains that bind FAP provided herein. In some embodiments, a multispecific antibody comprises two antigen binding domains that bind CD40. In some embodiments, each antigen binding domain that binds CD40 is a Fab. In some embodiments, the two Fab antigen binding domains are the same. In some embodiments, a multispecific antibody comprises one antigen binding domain that binds FAP. In some embodiments, the antigen binding domain that binds FAP is a scFv.

In some embodiments, the multispecific antibody provided herein comprises two Fab antigen binding fragments of an antibody that binds to CD40, one scFv antigen binding domain that binds to FAP, and an Fc domain. In some embodiments, the heavy chain (CH1) of each of the Fabs that bind to CD40 are linked via their C-termini to the hinge region of the Fc domain. As such, the heavy chains of the multispecific binding domain includes the heavy chain variable region and a heavy chain constant region, in which the heavy chain constant region includes the CH1 and the Fc comprising a hinge domain, CH2 domain and CH3 domain. In particular embodiments, the Fc is a heterodimeric Fc comprising a different first Fc polypeptide chain and second Fc polypeptide chain, each comprising a hinge-CH2-CH3, in which the interface of one of the CH3 domains is modified or altered to promote the formation of the trivalent bispecific antibody. In some embodiments, the scFv that binds to FAP is linked with its N-terminus to the C-terminus of the CH3 of one of the Fc polypeptide chains. In some embodiments, the linkage is via a peptide linker. In some embodiments, the amino acid sequence of the peptide linker comprises or consists of (GGGGS)n. In some such embodiments, the spacer has between 5 and 30 amino acids. In some embodiments, n is between 1 and 6 (inclusive). In some embodiments, n=4 and the linker is set forth as GGGGSGGGGSGGGGSGGGGS (SEQ ID NO:54). In some embodiments, the peptide linker is set forth as GGSGGGGSGGGGSGGGGS (SEQ ID NO: 35).

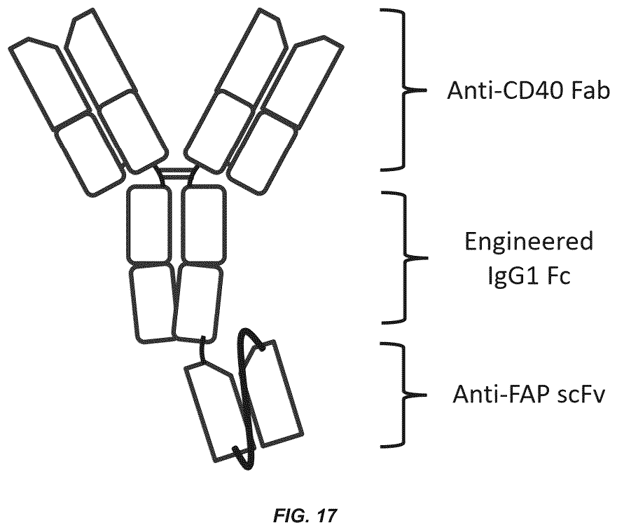

FIG. 17 provides an exemplary depiction of provided multispecific antibodies.

In some embodiments, the multispecific antibody is a CD40 agonist in the presence of FAP-expressing cells. In some embodiments, the multispecific antibody activates dendritic cells and macrophages in the presence of FAP-expressing cells.

In some embodiments, the multispecific antibody does not compete with CD40L for binding to CD40.

In some embodiments, the multispecific antibody comprises a post-translational modification. In some embodiments, the post-translational modifications may include amino acid modifications as a result of glycosylation, oxidation, deamidation, isomerization, glycation, formation of pyroglutamate, and deletion of C-terminal lysine or other amino acids, or a combination of the foregoing. Certain amino acids of a protein can be modified post-transcriptionally and the amino acid sequences provided herein include amino acids that contain a post-translational modification, e.g., glycosylation, oxidation, deamidation, isomerization, glycation, formation of pyroglutamate, and deletion of C-terminal lysine or other amino acids, or a combination of the foregoing.

In some embodiments, post-translational modifications can include glycosylation, oxidation of Met and Trp amino acids, deamidation of Asn amino acids, isomerization of Asp amino acids to isoaspartic acid (isoAsp), glycation, and presence of free thiols. In some embodiments, the multispecific antibody comprises at least one of the post-translational modifications listed in Example 19.

In some embodiments, the post-translational modification includes oxidation. In some embodiments, the post-translational modification includes oxidation of the heavy or light chains in accord with description in Example 19. In some embodiments, the post-translational modification includes oxidation on any of the amino acids of the first polypeptide, second polypeptide, or third polypeptide according to Table 14. In some embodiments, the oxidation is of methionine (Met) residues to form methionine sulfoxide or methionine sulfone. In some embodiments, the oxidation is of a tryptophan residue. In some embodiments, an oxidation product of tryptophan may include oxindolcalone (Oia) (4, 5, 6, or 7)-hydroxytryptophan, dioxindolcalanine (DiOia), kynurenine (Kyn) or N-formylkynurenine. In some embodiments, provided are compositions comprising proteins in which less than 10%, less than 9%, less than 8%, less than 7%, less than 6%, 5%, less than 4%, or less than 3% have oxidation of a methionine or tryptophan. In some embodiments, provided are compositions comprising proteins in which there less than 2% at any individual methionine residue has oxidation of the methionine. In some embodiments, provided are compositions comprising proteins in which less than 0.5%, such as about 0.1%, at any individual tryptophan residue has oxidation of the tryptophan (e.g., Trp94).

In some embodiments, the post-translational modification includes deamidation. In some embodiments, the post-translational modification includes deamidation of the heavy or light chains in accord with description in Example 19. In some embodiments, the post-translational modification includes deamidation on any of the amino acids of the first polypeptide, second polypeptide, or third polypeptide according to Table 15. In some embodiments, provided are compositions comprising proteins in which less than 10%, less than 9%, less than 8%, less than 7%, less than 6%, 5%, less than 4%, or less than 3% of the of the proteins have deamidation of a asparagine (Asn). In some embodiments, provided are compositions comprising proteins in which deamidation of a asparagine (Asn) represents less than 3% of the proteins in the composition.

In some embodiments, the post-translational modification includes isomerization and/or cyclic imide formation. In some embodiments, the post-translational modification includes isomerization and/or cyclic amide formation of the heavy or light chains in accord with description in Example 19. In some embodiments, the post-translational modification includes isomerization and/or cyclic imide formation on the first or second polypeptide according to Table 16. In some embodiments, provided are compositions comprising proteins in which less than 5%, less than 4%, less than 3%, less than 2%, or less than 1% have isomerization of an Asp.

In some embodiments, the post-translational modification includes the formation of free thiols (unpaired cysteines). In some embodiments, the post-translational modification includes formation of free thiols in accord with description in Example 19. In some embodiments, provided are compositions comprising proteins in which less than 5%, less than 4%, less than 3%, less than 2%, or less than 1% of proteins in the composition have a free thiol.

In some embodiments, the post-translational modification includes glycation of a lysine residue. In some embodiments, the post-translational modification includes glycation of the heavy or light chains in accord with description in Example 19. In some embodiments, provided are compositions comprising proteins in which the presence of a glycated lysine represents less than 20%, less than 17%, less than 15%, less than 13% or less than 10% of lysine residues of proteins in the composition.

In some embodiments, the multispecific antibody comprises a post-translational modification that is a post-translational modification of one or more amino acids from the N-terminus of the light chain and/or heavy chain. For heavy or light chains or their VH or VL domains disclosed herein that have an N-terminal glutamine (Q) or glutamic acid/glutamate (E), the N-terminal Q or E can be replaced by a pyro-glutamate. Accordingly, any VH or VL amino acid sequence disclosed herein having a Q or an E as N-terminal amino acid sequence should be understood to encompass those in which the Q or E is replaced by a pyro-glutamate. In some embodiments, provided are compositions comprising proteins in which at least about 90%, 91%, 92%, 93%, 94%, 95%, 96%, 97%, 98% or 99% of the proteins in the composition comprise an N-terminal VH or VL having an N-terminal E, and the remainder of the proteins comprise an N-terminal VH or VL having a pyroglutamate. In some of any embodiments, the N-terminus of the light chain (e.g., the first or second light chain) comprising the amino acid sequence set forth in SEQ ID NO: 44 undergoes a post-translational modification to become the amino acid sequence set forth in SEQ ID NO: 45. In some embodiments, the N-terminus of the heavy chain (e.g., the first or second polypeptide comprising the heavy chain) comprising the amino acid sequence set forth in SEQ ID NO: 46 undergoes a post-translational modification to become the amino acid sequence set forth in SEQ ID NO: 47.

In some embodiments, the multispecific antibody comprises a post-translational modification that is post-translational cleavage of one or more amino acids from the C-terminus of the heavy chain. In some embodiments, the post-translational modification is the cleavage of one or two amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. In some of any embodiments, the C-terminus of the heavy chain comprising the amino acid sequence set forth in SEQ ID NO:48 undergoes a post-translational cleavage to become the amino acid sequence set forth in SEQ ID NO:49. In some embodiments, a polypeptide or heavy chain comprising the amino acid sequence set forth in SEQ ID NO: 15 can undergo a post-translational cleavage at the C-terminus of the heavy chain to become the amino acid sequence set forth in SEQ ID NO: 43.

It is understood that any of the variations and modifications described above for polypeptides, such as heavy or light chains, of the present invention may be included in antibodies of the present invention.

### A. CD40

In some embodiments, the at least one antigen binding domain that binds CD40 in the provided multispecific antibodies is any described in Section II.

Provided herein is a multispecific antibody comprising at least one antigen binding domain that binds CD40, wherein at least one antigen binding domain comprises a heavy chain variable region (VH) comprising a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and a light chain variable region (VL) comprising a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

In some embodiments, the at least one antigen binding domain that binds CD40 of a provided multispecific antibody is one in which the heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

In some embodiments, the at least one antigen binding domain that binds CD40 of a provided multispecific antibody is one in which the heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

In some embodiments, an antigen binding domain that binds CD40 comprises a heavy chain variable region (VH) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 13; and a light chain variable region (VL) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 14. In some embodiments, a VH sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, and a VL sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain comprising that sequence retains the ability to bind to CD40. In some embodiments, a total of 1 to 10 amino acids have been substituted, inserted and/or deleted in SEQ ID NO: 13. In some embodiments, a total of 1 to 10 amino acids have been substituted, inserted and/or deleted in SEQ ID NO: 14. In some embodiments, substitutions, insertions, or deletions occur in regions outside the CDRs (that is, in the FRs). In some embodiments, the antigen binding domain that binds CD40 comprises the VH sequence of SEQ ID NO: 13 and the VL sequence of SEQ ID NO: 14. In some embodiments, the anti-CD40 antibody comprises the VH sequence of SEQ ID NO: 13 and the VL sequence of SEQ ID NO: 14, including post-translational modifications of one or both sequences.

In some embodiments, a multispecific antibody comprises at least one antigen binding domain that binds CD40, wherein the antigen binding domain comprises a heavy chain variable region comprises the amino acid sequence of SEQ ID NO: 13, and a light chain variable region comprises the amino acid sequence of SEQ ID NO: 14. In some embodiments, the multispecific antibody that binds CD40 comprises two antigen binding domains that bind CD40, wherein each antigen binding domain comprises a heavy chain variable region comprises the amino acid sequence of SEQ ID NO: 13, and a light chain variable region comprises the amino acid sequence of SEQ ID NO: 14.

In some embodiments, the first and second antigen binding domains that bind CD40 each comprise a Fab. In some embodiments, the Fab comprises: (i) a Fab heavy chain comprising a heavy chain variable region and a heavy chain constant region (CH1), and (ii) a light chain comprising a light chain variable region (VL) fused to a light chain constant region. In some embodiments, each Fab is the same.

In some embodiments, the CH1 is an IgG1, IgG2, IgG3, or IgG4 CH1. In some embodiments, each CH1 is an IgG1 CH1. In some embodiments, the CH1 comprises an amino acid sequence of SEQ ID NO: 50. In some embodiments, each Fab heavy chain comprises an amino acid sequence at least 85%, at least 90%, or at least 95% identical to the amino acid sequence of SEQ ID NO: 53. In some embodiments, each Fab heavy chain comprises the amino acid sequence of SEQ ID NO: 53.

In some embodiments, the light chain comprises any of the light chain variable regions described herein and a light chain constant region. In some embodiments, the light chain comprises the light chain variable region set forth in SEQ ID NO: 14. In some embodiments, the light chain constant region comprises an amino acid sequence at least 85%, at least 90%, or at least 95% identical to the amino acid sequence of SEQ ID NO: 57. In some embodiments, the light chain constant region set forth in SEQ ID NO: 57. In some embodiments, the light chain comprises the light chain variable region set forth in SEQ ID NO: 14 and the light chain constant region set forth in SEQ ID NO: 57.

In some embodiments, the light chain comprises an amino acid sequence at least 85%, at least 90%, or at least 95% identical to the amino acid sequence of SEQ ID NO: 16. In some embodiments, the light chain comprises the amino acid sequence of SEQ ID NO: 16.

In some embodiments, the Fab comprises the Fab heavy chain comprising the amino acid sequence of SEQ ID NO: 53 and the light chain comprising the amino acid sequence of SEQ ID NO: 16.

### B. FAP

Among the provided multispecific antibodies are antibodies that comprise at least one antigen binding domain specifically binds FAP, such as human FAP. Such antibodies include, but are not limited to, monoclonal antibodies, humanized antibodies, chimeric antibodies, mouse antibodies, human antibodies, and antibodies comprising the heavy chain and/or light chain CDRs discussed herein. In some embodiments, the antibody binds to human FAP. In some embodiments, the antibody binds to human FAP comprising the amino acid sequence of SEQ ID NO: 37.

In some embodiments, an antigen binding domain that binds FAP comprises a heavy chain variable region and a light chain variable region. In some embodiments, an antibody comprising at least one antigen binding domain that binds FAP comprises at least one heavy chain comprising a heavy chain variable region, and at least one light chain comprising a light chain variable region. In some embodiments, an antigen binding domain that binds FAP is a single-chain Fv (scFv). As used herein, a single-chain Fv (scFv), or any other antibody that comprises, for example, a single polypeptide chain comprising all six CDRs (three heavy chain CDRs and three light chain CDRs) is considered to have a heavy chain variable region and a light chain variable region. In some embodiments, the heavy chain variable region is the region of the anti-FAP antibody that comprises the three heavy chain CDRs. In some embodiments, the light chain variable region is the region of the anti-FAP antibody that comprises the three light chain CDRs.

In some embodiments, an antigen binding domain that binds FAP comprises at least one, two, three, four, five, or six CDRs selected from a heavy chain complementarity determining region 1 (HCDR1) comprising an amino acid sequence selected from SEQ ID NO: 17 or 23; a heavy chain complementarity determining region 2 (HCDR2) comprising an amino acid sequence selected from SEQ ID NO: 18 or 24; a heavy chain complementarity determining region 3 (HCDR3) comprising the amino acid sequence of SEQ ID NO: 19 or 25; a light chain complementarity determining region 1 (LCDR1) comprising an amino acid sequence selected from SEQ ID NO: 20 or 26; a light chain complementarity determining region 2 (LCDR2) comprising an amino acid sequence selected from SEQ ID NO: 21 or 27; and a light chain complementarity determining region 3 (LCDR3) comprising the amino acid sequence of SEQ ID NO: 22 or 28.

In some embodiments, the antigen binding domain that binds FAP comprises six CDRs including a HCDR1 comprising an amino acid sequence selected from SEQ ID NO: 17 or 23; a HCDR2 comprising an amino acid sequence selected from SEQ ID NO: 18 or 24; a HCDR3 comprising the amino acid sequence of SEQ ID NO: 19 or 25; a LCDR1 comprising an amino acid sequence selected from SEQ ID NO: 20 or 26; a LCDR2 comprising an amino acid sequence selected from SEQ ID NO: 21 or 27; and a LCDR3 comprising the amino acid sequence of SEQ ID NO: 22 or 28.

In some embodiments, an antigen binding domain that binds FAP comprises at least once, two, three, four, five, or six CDRs selected from a HCDR1 comprising the amino acid sequence of SEQ ID NO: 17; a HCDR2 comprising the amino acid sequence of SEQ ID NO: 18; a HCDR3 comprising the amino acid sequence of SEQ ID NO: 19; a LCDR1 comprising an amino acid sequence selected from SEQ ID NO: 20; a LCDR2 comprising an amino acid sequence selected from SEQ ID NO: 21; and a LCDR3 comprising the amino acid sequence of SEQ ID NO: 22.

In some embodiments, an antigen binding domain that binds FAP comprises at least one, two, three, four, five, or six CDRs selected from a HCDR1 comprising the amino acid sequence of SEQ ID NO: 23; a HCDR2 comprising the amino acid sequence of SEQ ID NO: 24; a HCDR3 comprising the amino acid sequence of SEQ ID NO: 25; a LCDR1 comprising an amino acid sequence selected from SEQ ID NO: 26; a LCDR2 comprising an amino acid sequence selected from SEQ ID NO: 27; and a LCDR3 comprising the amino acid sequence of SEQ ID NO: 28.

In some embodiments, the antigen binding domain that binds FAP comprises six CDRs including a HCDR1 comprising the amino acid sequence of SEQ ID NO: 17; a HCDR2 comprising the amino acid sequence of SEQ ID NO: 18; a HCDR3 comprising the amino acid sequence of SEQ ID NO: 19; a LCDR1 comprising an amino acid sequence selected from SEQ ID NO: 20; a LCDR2 comprising an amino acid sequence selected from SEQ ID NO: 21; and a LCDR3 comprising the amino acid sequence of SEQ ID NO: 22.

In some embodiments, the antigen binding domain that binds FAP comprises six CDRs including a HCDR1 comprising the amino acid sequence of SEQ ID NO: 23; a HCDR2 comprising the amino acid sequence of SEQ ID NO: 24; a HCDR3 comprising the amino acid sequence of SEQ ID NO: 25; a LCDR1 comprising an amino acid sequence selected from SEQ ID NO: 26; a LCDR2 comprising an amino acid sequence selected from SEQ ID NO: 27; and a LCDR3 comprising the amino acid sequence of SEQ ID NO: 28.

In some embodiments, an antigen binding domain that binds FAP comprises a heavy chain variable region comprising a heavy chain complementarity determining region (HCDR) 1 comprising the amino acid sequence of SEQ ID NO: 17 or 23, a HCDR2 comprising the amino acid sequence of SEQ ID NO: 18 or 24, and a HCDR3 comprising the amino acid sequence of SEQ ID NO: 19 or 25, and a light chain variable region comprising a LCDR1 comprising the amino acid sequence of SEQ ID NO: 20 or 26, a LCDR2 comprising the amino acid sequence of SEQ ID NO: 21 or 27, and a LCDR3 comprising the amino acid sequence of SEQ ID NO: 22 or 28.

In some embodiments, an antigen binding domain that binds FAP comprises heavy chain variable region comprising a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19; and a light chain variable region comprising a LCDR1 comprising the amino acid sequence of SEQ ID NO: 20, a LCDR2 comprising the amino acid sequence of SEQ ID NO: 21, and a LCDR3 comprising the amino acid sequence of SEQ ID NO: 22.

In some embodiments, an antigen binding domain that binds FAP comprises a heavy chain variable region comprising a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25; and a light chain variable region comprising a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

In some embodiments, any of the six anti-FAP CDRs as defined by Kabat can be combined as subparts with any of the anti-FAP CDRs as defined by IMGT, for a total of six CDRs in a construct. Thus, in some embodiments, two CDRs defined by Kabat (for example, HCDR1 and HCDR2) can be combined with four CDRs as defined by IMGT (HCDR3, LCDR1, LCDR2, and LCDR3). In some embodiments, two or fewer residues in one or more of the CDRs can be replaced to obtain a variant thereof. In some embodiments, two or fewer residues can be replaced in 1, 2, 3, 4, 5, or 6 of the CDRs.

In some embodiments, the antibody comprising at least one antigen binding domain that binds FAP comprises (I) a heavy chain variable region (VH) comprising at least one, at least two, or all three VH CDR sequences selected from (i) an HCDR1 comprising an amino acid sequence selected from SEQ ID NO: 17 or 23; (ii) an HCDR2 comprising an amino acid sequence selected from SEQ ID NO: 18 or 24; (iii) an HCDR3 comprising the amino acid sequence of SEQ ID NO: 19 or 25; and (II) a VL comprising at least one, at least two, or all three VL CDR sequences selected from (iv) an LCDR1 comprising an amino acid sequence selected from SEQ ID NO: 20 or 26; (v) an LCDR2 comprising an amino acid sequence selected from SEQ ID NO: 21 or 27; and (vi) an LCDR3 comprising the amino acid sequence of SEQ ID NO: 22 or 28; wherein the VH comprises a sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 29 or 31, and the VL comprises a sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 30 or 32.

In some embodiments, an antibody comprising at least one antigen binding domain that binds FAP comprises a heavy chain variable region (VH) sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO:29 or 31. In some embodiments, a VH sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain comprising that sequence retains the ability to bind FAP. In some embodiments, a total of 1 to 10 amino acids have been substituted, inserted and/or deleted in SEQ ID NO: 29 or 31. In some embodiments, substitutions, insertions, or deletions occur in regions outside the CDRs (that is, in the framework regions (FRs)). In some embodiments, the antigen binding domain that binds FAP comprises the VH sequence of SEQ ID NO: 29. In some embodiments, the antigen binding domain that binds FAP comprises the VH sequence of SEQ ID NO: 29, including post-translational modifications of that sequence. In some embodiments, the antigen binding domain that binds FAP comprises the VH sequence of SEQ ID NO: 31. In some embodiments, the antigen binding domain that binds FAP comprises the VH sequence of SEQ ID NO: 31, including post-translational modifications of that sequence.

In some embodiments, an antibody comprising at least one antigen binding domain that binds FAP is provided, wherein the antibody comprises a light chain variable region (VL) having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 30 or 32. In some embodiments, a VL sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain comprising that sequence retains the ability to bind to FAP. In some embodiments, a total of 1 to 10 amino acids have been substituted, inserted and/or deleted in SEQ ID NO: 30 or 32. In some embodiments, the substitutions, insertions, or deletions occur in regions outside the CDRs (that is, in the FRs). In some embodiments, the antigen binding domain that binds FAP comprises the VL sequence of SEQ ID NO: 30. In some embodiments, the antigen binding domain that binds FAP comprises the VL sequence of SEQ ID NO: 30, including post-translational modifications of that sequence. In some embodiments, the antigen binding domain that binds FAP comprises the VL sequence of SEQ ID NO: 32. In some embodiments, the antigen binding domain that binds FAP comprises the VL sequence of SEQ ID NO: 32, including post-translational modifications of that sequence.

In some embodiments, an antibody that comprise at least one antigen binding domain that binds FAP comprises a heavy chain variable region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 29 or 31; and a light chain variable region (VL) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 30 or 32. In some embodiments, a VH sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, and a VL sequence having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but an antigen binding domain that binds FAP comprising that sequence retains the ability to bind to FAP. In some embodiments, a total of 1 to 10 amino acids have been substituted, inserted and/or deleted in SEQ ID NO: 29 or 31. In some embodiments, a total of 1 to 10 amino acids have been substituted, inserted and/or deleted in SEQ ID NO: 30 or 32. In some embodiments, substitutions, insertions, or deletions occur in regions outside the CDRs (that is, in the FRs). In some embodiments, antigen binding domain that binds FAP comprises the VH sequence of SEQ ID NO: 29 or 31 and the VL sequence of SEQ ID NO: 30 or 32. In some embodiments, antigen binding domain that binds FAP comprises the VH sequence of SEQ ID NO: 29 and the VL sequence of SEQ ID NO: 30, including post-translational modifications of one or both sequences. In some embodiments, antigen binding domain that binds FAP comprises the VH sequence of SEQ ID NO: 31 and the VL sequence of SEQ ID NO: 32, including post-translational modifications of one or both sequences.

In some embodiments, the antigen binding domain that binds FAP is a Fab, Fab′, F(ab′)₂, Fd, Fv, single-chain Fv (scFv) or disulfide-linked Fv (sdFv). In some embodiments, the antigen binding domain is a single-chain Fv (scFv).

In some embodiments, the scFv is in a VH-VL orientation. In some embodiments, the scFv is in a VL-VH orientation. In some embodiments, there is present a peptide linker between the VH and VL sequences. In some embodiments, the amino acid sequence of the linker comprises or consists of (GGGGS) n. In some such embodiments, the spacer has between 5 and 30 amino acids. In some embodiments, n is between 1 and 6 (inclusive). In some embodiments, n=4 and the linker is set forth as GGGGSGGGGSGGGGSGGGGS (SEQ ID NO:54). In some embodiments, the peptide linker is set forth as GGSGGGGSGGGGSGGGGS (SEQ ID NO: 35).

In some embodiments, the antigen binding domain that binds FAP is a scFv. In some such embodiments, the antigen binding domain that binds FAP comprises (I) a VH comprising at least one, at least two, or all three VH CDR sequences selected from (i) an HCDR1 comprising an amino acid sequence selected from SEQ ID NO: 17 or 23; (ii) an HCDR2 comprising an amino acid sequence selected from SEQ ID NO: 18 or 24; (iii) an HCDR3 comprising the amino acid sequence of SEQ ID NO: 19 or 25; and (II) a VL comprising at least one, at least two, or all three VL CDR sequences selected from (iv) an LCDR1 comprising an amino acid sequence selected from SEQ ID NO: 20 or 26; (v) an LCDR2 comprising an amino acid sequence selected from SEQ ID NO: 21 or 27; and (vi) an LCDR3 comprising the amino acid sequence of SEQ ID NO: 22 or 28; wherein the scFv comprises a sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 33.

In some embodiments, the scFv comprises two cysteine substitutions that form a disulfide-linked Fv. In some embodiments, the scFv comprises one or more substitutions selected from VH-G44C and VL-Q100C. In some embodiments, the scFv comprises a G44C substitution in the VH and a Q100C substitution in the VL, wherein substitution positions are according to Kabat. In some instances, the cysteine substitutions at these two amino acid positions in the scFv form a disulfide bond between the VH and VL domains.

In some embodiments, an antigen binding domain that binds FAP is an scFv that has an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 33. In some embodiments, an antigen binding domain that binds FAP is an scFv comprising the amino acid sequence of SEQ ID NO: 33.

In various embodiments, the antibody is a monoclonal antibody.

In various embodiments, an antibody comprising an antigen binding domain that binds FAP may comprise any of the following properties, singly or in combination. In some embodiments, the antibody binds to human FAP with an affinity (K_{D}) between 0.5 and 10 nM, or 0.5 and 7 nM, or 1 and 5 nM, as determined by surface plasmon resonance. In some embodiments, the antibody binds to cynomolgus monkey FAP with a K_{D} between 0.5 and 10 nM, or 0.5 and 7 nM, or 1 and 5 nM, as determined by surface plasmon resonance. In some embodiments, the antibody binds human FAP expressed on the surface of cells with an EC50 of between 0.5 and 10 nM, or 0.5 and 7 nM, or 1 and 5 nM.

In some embodiments, the antibody binds human FAP. In some embodiments, the human FAP comprises the amino acid sequence of SEQ ID NO: 37.

### C. Constant Domain, e.g., Fc Domain

In some embodiments, the multispecific antibody is a full length antibody, such as a full length IgG1 antibody. In some embodiments, the full-length IgG1 antibody is a full length IgG1, λ (lambda), κ (kappa) antibody or IgG1, κ (kappa), κ (kappa) antibody. In some embodiments, the full-length antibody includes an Fc region, e.g. comprising at least a hinge region, a CH2 domain, and a CH3 domain. In some embodiments, the bispecific antibody is a dimer formed by polypeptides, each containing an Fc region.

In various embodiments, a multispecific antibody is provided, wherein the multispecific antibody comprises a first heavy chain comprising the first heavy chain variable region fused to a first heavy chain constant region; a first light chain comprising the first light chain variable region fused to a first light chain constant region; a second heavy chain comprising the second heavy chain variable region fused to a second heavy chain constant region; a second light chain comprising the second light chain variable region fused to a second light chain constant region; and an scFv fused to the C-terminus of the first heavy chain constant region. In some embodiments, the scFv is fused to the C-terminus of the first heavy chain constant region via a linker. In some embodiments, the multispecific antibody comprises i) a full-length antibody comprising a first heavy chain comprising the first heavy chain variable region fused to a first heavy chain constant region; a first light chain comprising the first light chain variable region fused to a first light chain constant region; a second heavy chain comprising the second heavy chain variable region fused to a second heavy chain constant region; and a second light chain comprising the second light chain variable region fused to a second light chain constant region; and ii) an scFv fused to the C-terminus of the first heavy chain constant region of the full-length antibody via a linker. In some such embodiments, the first heavy chain variable region and the first light chain variable region together form an antigen binding region that binds CD40, and the second heavy chain variable region and the second light chain variable region together form an antigen binding region that binds CD40, and the scFv binds FAP. In some embodiments, the two antigen binding regions that bind CD40 have the same amino acid sequences.

In some embodiments, the first heavy chain constant region and the second heavy chain constant region are the same. In some embodiments, the first heavy chain constant region and the second heavy chain constant region are different.

In some embodiments, the scFv is fused to the C-terminus of the first heavy chain constant region. In some embodiments, the scFv is fused directly to the C-terminus of the first heavy chain constant region. In some embodiments, the scFv is fused indirectly to the C-terminus of the first heavy chain constant region via a linker. In some embodiments, the linker is an amino acid linker. In some embodiments, the linker comprises glycine and/or serine residues. In some embodiments, the linker comprises the amino acid sequence of SEQ ID NO: 35.

In some embodiments, each heavy chain constant region of each heavy chain polypeptide comprises the heavy chain constant region 1 (CH1), such as any CH1 described herein, and an Fc polypeptide chain. In some embodiments, each Fc is an Fc region described herein in Section IV.E.

In some embodiments, the Fc polypeptides of the heavy chain constant region of the first and second heavy chain constant regions are different. In some embodiments, the Fc polypeptide chain of the first heavy chain constant region comprises at least one first heterodimerization mutations and the Fc polypeptide chain of the second heavy chain constant region comprises at least one second heterodimerization mutation. In some such embodiments, the Fc is formed by Fc polypeptides that are mutated or modified to promote heterodimerization in which different polypeptides can be dimerized to yield a heterodimer. Thus, in some embodiments, the dimer is a heterodimer in which two heavy chain polypeptides of the multispecific antibody are different. Exemplary modifications to promote heterodimerization are known, including any as described below.

In provided embodiments, a multispecific antibody provided herein comprises a heterodimeric Fc that facilitates interactions of two different heavy chain polypeptides in which the first heavy chain polypeptide includes the heavy chain of a first Fab that binds CD40 and a first Fc polypeptide chain with heterodimeric mutations and the second heavy chain polypeptide includes the heavy chain of the second Fab that binds CD40 and a second Fc polypeptide chain with heterodimeric mutations, in which one of the first Fc polypeptide chain or second Fc polypeptide chain is linked at its C-terminus to the scFv that binds FAP. In some embodiments the first polypeptide chain of the heterodimeric Fc-region comprises a first CH3 region, and the second polypeptide chain of the heterodimeric Fc region comprises a second CH3 region, wherein the sequences of the first and second CH3 regions are different and are such that the heterodimeric interaction between said first and second CH3 regions is stronger than each of the homodimeric interactions of said first and second CH3 regions.

In some embodiments, the first heavy chain constant region and the second heavy chain constant region are different. In some embodiments, the first heavy chain constant region and the second heavy chain constant region form a heterodimer. Methods and variants for heterodimerization also include those described in published international PCT App. WO2014/145806, including “knobs and holes” mutations (also called “skew” variants), mutations that relate to “electrostatic steering” or “charge pairs,” and pI variants. Heterodimeric variants also include any as described in U.S. published Appl. No. US2012/0149876 or US2018/011883.

In some embodiments, modifications include introduction of a protuberance (knob) into the first or second Fc polypeptide chain (e.g., Fc polypeptide chain of the first or second heavy chain constant region) and a cavity (hole) into the other of the first or second Fc polypeptide chain (e.g., Fc polypeptide chain of the first or second heavy chain constant region) such that the protuberance is positionable in the cavity to promote complexing of the Fes of the first and second heavy chain constant regions. Amino acids targeted for replacement and/or modification to create protuberances or cavities in a polypeptide are typically interface amino acids that interact or contact with one or more amino acids in the interface of another polypeptide.

In some embodiments, the Fc polypeptide chain of the first heavy chain constant region is modified to contain protuberance (hole) amino acids include replacement of a native or original amino acid with an amino acid that has at least one side chain which projects from the interface of the Fc of the first heavy chain constant region and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, the Fc of the second heavy chain constant region is modified to contain a cavity (hole) is one that includes replacement of a native or original amino acid with an amino acid that has at least one side chain that is recessed from the interface of the Fc of the second heavy chain constant region and thus is able to accommodate a corresponding protuberance from the interface of the Fc of the first heavy chain constant region. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine(S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9:617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9:617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, to promote heterodimerization of both polypeptides of the Fc and/or constant region, the heterodimer contain paired or complementary amino acid modifications. Exemplary paired amino acid modification of polypeptides of an Fc fusion are set forth in Table 1. The Table depicts the mutations of a first Fc polypeptide chain and a second Fc polypeptide chain for exemplification purposes. However, it is understood that the recited mutations can be reversed so long as the paired mutations are present, such that the recited mutations of the first Fc polypeptide chain can be made in a second Fc polypeptide chain, and the recited mutations of the second Fc polypeptide chain can be made in a first Fc polypeptide chain.

TABLE 1
Paired amino acids of Heterodimeric Fc

| First Fc polypeptide chain | Second Fc polypeptide chain |
|---|---|
| T366W | T366S/L368W/Y407V |
| T366W | T366S/L368A/Y407V |
| T366W/S354C | T366S/L368A/Y407V/Y349C |
| S364H/F405A | Y349T/Y349F |
| T350V/L351Y/F405A/Y407V | T350V/T366L/K392L/T394W |
| T350V/L351Y/S400E/F405A/ | T350V/T366L/N390R/K392M/ |
| Y407V | T394W |
| T350V/L351Y/S400E/F405A/ | T350V/T366L/N390R/K392L/ |
| Y407V | T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/399V/F405T/S354C |
| K370E/K409W | E357N/D399V/F405T |

In some embodiments, the first heavy chain constant region and/or the second heavy chain constant region comprise one or more heterodimerization mutation. In some embodiments, the heterodimerization mutations comprises one or more of T350V, L351Y, T366W, T366S, T366L, L368A, N390R, K392M, K392L, T394W, S400E, F405A, and/or Y407V, wherein mutation positions are according to Kabat. See, e.g., Escobar-Cabrera et al., Antibodies (Basel), 6(2): 7 (2017), and WO 2013/166594.

In some embodiments, the first heavy chain constant region comprises at least one first heterodimerization mutation and the second heavy chain constant region comprises at least one second heterodimerization mutation. In some embodiments, at least one first heterodimerization mutation comprises T366W and at least one second heterodimerization mutation comprises one or more of T366S, L368A, and/or Y407V, wherein mutation positions are according to Kabat. In some embodiments, at least one first heterodimerization mutation comprises one or more of T366S, L368A, and/or Y407V and at least one second heterodimerization mutation comprises T366W, wherein mutation positions are according to Kabat. In some embodiments, at least one first heterodimerization mutation comprises T366W and at least one second heterodimerization mutation comprises T366S, L368A, and Y407V, wherein mutation positions are according to Kabat. In some embodiments, at least one first heterodimerization mutation comprises T366S, L368A, and Y407V and at least one second heterodimerization mutation comprises T366W, wherein mutation positions are according to Kabat.

In some embodiments, at least one first heterodimerization mutation comprises one or more of T350V, L351Y, S400E, F405A, and/or Y407V and at least one second heterodimerization mutation comprises one or more of T350V, T366L, N390R, K392M, K392L, and/or T394W, wherein mutation positions are according to Kabat. In some embodiments, at least one first heterodimerization mutation comprises one or more of T350V, T366L, N390R, K392M, K392L, and/or T394W and at least one second heterodimerization mutation comprises one or more of T350V, L351Y, S400E, F405A, and/or Y407V, wherein mutation positions are according to Kabat. In some embodiments, at least one first heterodimerization mutation comprises T350V, L351Y, F405A, and Y407V and at least one second heterodimerization mutation comprises T350V, T366L, K392L, and T394W, wherein mutation positions are according to Kabat. In some embodiments, at least one first heterodimerization mutation comprises one or more of T350V, T366L, K392L, and T394W and at least one second heterodimerization mutation comprises one or more of T350V, L351Y, F405A, and Y407V, wherein mutation positions are according to Kabat.

In some embodiments, the multispecific antibody is an IgG1, IgG2, IgG3, or IgG4. In some embodiments, a multispecific antibody is an IgG1. Exemplary constant regions and modifications of constant regions are further described in Section IV.E, below.

In some embodiments, each Fc polypeptide chain is effectorless. In some embodiments, each Fc polypeptide chain comprises L234A, L235A, and/or D265S substitutions, wherein substitution positions are according to Kabat. In some embodiments, each Fc polypeptide chain comprises L234A, L235A, and D265S substitutions, wherein substitution positions are according to Kabat.

In some embodiments, the first Fc polypeptide chain (e.g., Fc polypeptide chain of the first heavy chain constant region) comprises an amino acid sequence at least 85%, at least 90%, or at least 95% identical to the amino acid sequence of SEQ ID NO: 51 or SEQ ID NO: 52, and wherein the second Fc polypeptide chain (e.g., Fc polypeptide chain of the second heavy chain constant region) comprises an amino acid sequence at least 85%, at least 90%, or at least 95% identical to the amino acid sequence of the other of SEQ ID NO: 51 or SEQ ID NO: 52. In some embodiments, the first Fc polypeptide chain comprises the amino acid sequence of SEQ ID NO: 51 or SEQ ID NO: 52, and wherein the second Fc polypeptide chain comprises the amino acid sequence of the other of SEQ ID NO: 51 or SEQ ID NO: 52.

In some embodiments, the first Fc polypeptide chain comprises an amino acid sequence at least 85%, at least 90%, or at least 95% identical to the amino acid sequence of SEQ ID NO: 51. In some embodiments, the first Fc polypeptide chain comprises the amino acid sequence of SEQ ID NO: 51. In some embodiments, the first Fc polypeptide chain comprises an amino acid sequence at least 85%, at least 90%, or at least 95% identical to the amino acid sequence of SEQ ID NO: 52. In some embodiments, the first Fc polypeptide chain comprises the amino acid sequence of SEQ ID NO: 52.

In some embodiments, the second Fc polypeptide chain comprises an amino acid sequence at least 85%, at least 90%, or at least 95% identical to the amino acid sequence of SEQ ID NO: 51. In some embodiments, the second Fc polypeptide chain comprises the amino acid sequence of SEQ ID NO: 51. In some embodiments, the second Fc polypeptide chain comprises an amino acid sequence at least 85%, at least 90%, or at least 95% identical to the amino acid sequence of SEQ ID NO: 52. In some embodiments, the second Fc polypeptide chain comprises the amino acid sequence of SEQ ID NO: 52.

### D. Exemplary Formats

In some embodiments, a multispecific antibody comprises: a) a first and second antigen binding domains that binds CD40, and each is a Fab comprising: (1) a Fab heavy chain comprising a heavy chain variable region and a heavy chain constant region (CH1), and (2) a light chain comprising a light chain variable region and a light chain constant region, wherein each Fab is the same; and b) an Fc that is a heterodimeric Fc containing a first Fc polypeptide chain and second Fc polypeptide chain, each comprising a hinge, CH2 and CH3 domain, wherein the N-terminus of the Fc of the first Fc polypeptide is linked to the C-terminus of the CH1 of one Fab heavy chain and the N-terminus of the second Fc polypeptide is linked to the C-terminus of the CH1 of the second Fab heavy chain; and c) a third antigen binding domain that binds to FAP that is an scFv, such as any described herein, that is fused to the C-terminus of the Fc of one of the Fc polypeptide chains. In such embodiments, the CH3 domains of the first and second Fc polypeptide chains contain different heterodimerization mutations to promote formation of a heterodimer between the Fc polypeptide chains.

In some embodiments, each Fab comprises the Fab heavy chain comprising the amino acid sequence of SEQ ID NO: 53 and the light chain comprising the amino acid sequence of SEQ ID NO: 16. In some embodiments, the scFv is a disulfide-stabilized scFv comprising a G44C substitution in the VH and a Q100C substitution in the VL. In some embodiments, the scFv comprises the amino acid sequence set forth in SEQ ID NO:33. In some embodiments, the first and second Fc polypeptides are set forth in SEQ ID NOS: 51 and 52.

In some embodiments, the multispecific antibody comprises a first polypeptide, a second polypeptide, and a third polypeptide.

In some embodiments, the first polypeptide comprises, from N-terminus to C-terminus: a first heavy chain variable region, a first heavy chain constant region and an scFv comprising a third heavy chain variable region and a third light chain variable region. In some embodiments, the first polypeptide comprises, from N-terminus to C-terminus: a first heavy chain variable region, a first heavy chain constant region and an scFv comprising a third heavy chain variable region and a third light chain variable region, where a linker connects the C-terminus of the first heavy chain constant region to the N-terminus of the scFv. In some embodiments, the second polypeptide comprises, from N-terminus to C-terminus: a second heavy chain variable region and a second heavy chain constant region. In some embodiments, the third polypeptide comprises a first light chain variable region and a first light chain constant region. In such embodiments, the first heavy chain constant region and the second heavy chain constant region are different, in which each includes a CH1, hinge, CH2 and CH3 domain and in which the CH3 domains of the first and second heavy chains contain different heterodimerization mutations to promote formation of a heterodimer between the Fc polypeptide chains. The first heavy chain variable region and the CH1 domain of the first heavy chain constant region are associated with a copy of the third polypeptide comprising the first light chain variable region and light chain constant region. The second heavy chain variable region and the CH1 domain of the second heavy chain constant region also are associated with a copy of the third polypeptide comprising the first light chain variable region and light chain constant regions.

In some embodiments, the multispecific antibody comprises a first polypeptide, a second polypeptide, and a third polypeptide, wherein: the first polypeptide comprises, from N-terminus to C-terminus: the Fab heavy chain of the first binding domain, one of the first Fc polypeptide chain or second Fc polypeptide chain, and the scFv of the third binding domain; the second polypeptide comprises, from N-terminus to C-terminus: the Fab heavy chain of the second binding domain and the other of the first and second Fc polypeptide chain; and the third polypeptide comprises the first light chain. In some embodiments, the ratio of the first polypeptide to the second polypeptide to the third polypeptide is about 1:1:2. In some embodiments, the ratio of the first polypeptide to the second polypeptide to the third polypeptide is 1:1:2.

In some embodiments, the first polypeptide comprises, from N-terminus to C-terminus: the Fab heavy chain of the first binding domain comprising the amino acid sequence of SEQ ID NO: 53, a first Fc polypeptide chain comprising the amino acid sequence of SEQ ID NO: 52, and the scFv of the third binding domain comprising the amino acid sequence of SEQ ID NO: 33. In some embodiments, the scFv is linked to the C-terminus of the Fv polypeptide chain via a peptide linker, such as set forth in SEQ ID NO: 54. In some embodiments, the second polypeptide comprises, from N-terminus to C-terminus: the Fab heavy chain of the second binding domain comprising the amino acid sequence of SEQ ID NO: 53, and a second Fc polypeptide chain comprising the amino acid sequence of SEQ ID NO: 51. In some embodiments, the third polypeptide comprises the light chain comprising the amino acid sequence of SEQ ID NO: 16.

In some embodiments, the ratio of the first polypeptide to the second polypeptide to the third polypeptide is about 1:1:2. In some embodiments, the ratio of the first polypeptide to the second polypeptide to the third polypeptide is 1:1:2.

In some embodiments, the first polypeptide of the multispecific antibody comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 34. In some embodiments, the first polypeptide having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but a multispecific antibody comprising that sequence retains the ability to bind to CD40 and FAP. In some embodiments, a total of 1 to 10 amino acids have been substituted, inserted and/or deleted in SEQ ID NO: 34. In some embodiments, substitutions, insertions, or deletions occur in regions outside the CDRs (that is, in the FRs). In some embodiments, the first polypeptide comprises the amino acid sequence of SEQ ID NO: 34. In some embodiments, the multispecific antibody comprises the first polypeptide sequence of SEQ ID NO: 34. In some embodiments, the multispecific antibody comprises the first polypeptide sequence of SEQ ID NO: 34, including post-translational modifications.

In some embodiments, the second polypeptide of the multispecific antibody comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 15. In some embodiments, the second polypeptide having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but a multispecific antibody comprising that sequence retains the ability to bind to CD40 and FAP. In some embodiments, a total of 1 to 10 amino acids have been substituted, inserted and/or deleted in SEQ ID NO: 15. In some embodiments, substitutions, insertions, or deletions occur in regions outside the CDRs (that is, in the FRs). In some embodiments, the second polypeptide comprises the amino acid sequence of SEQ ID NO: 15. In some embodiments, the multispecific antibody comprises the second polypeptide sequence of SEQ ID NO: 15. In some embodiments, the multispecific antibody comprises the second polypeptide sequence of SEQ ID NO: 15, including post-translational modifications.

In some embodiments, the third polypeptide of the multispecific antibody comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 16. In some embodiments, the third polypeptide having at least 85%, 86%, 87%, 88%, 89%, 90%, 91%, 92%, 93%, 94%, 95%, 96%, 97%, 98%, or 99% identity contains substitutions (for example, conservative substitutions), insertions, or deletions relative to the reference sequence, but a multispecific antibody comprising that sequence retains the ability to bind to CD40 and FAP. In some embodiments, a total of 1 to 10 amino acids have been substituted, inserted and/or deleted in SEQ ID NO: 16. In some embodiments, substitutions, insertions, or deletions occur in regions outside the CDRs (that is, in the FRs). In some embodiments, the third polypeptide comprises the amino acid sequence of SEQ ID NO: 16. In some embodiments, the multispecific antibody comprises the third polypeptide sequence of SEQ ID NO: 16. In some embodiments, the multispecific antibody comprises the third polypeptide sequence of SEQ ID NO: 16, including post-translational modifications.

In some embodiments, the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

Also provided herein is a multispecific antibody comprising two antigen binding domains that bind CD40 and one antigen binding domain that binds FAP, wherein the multispecific antibody comprises a first polypeptide, a second polypeptide, and a third polypeptide at a ratio of about 1:1:2, and wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

In some embodiments, the multispecific antibody comprises two antigen binding domains that bind CD40 and one antigen binding domain that binds FAP, wherein the multispecific antibody comprises a first polypeptide, a second polypeptide, and a third polypeptide at a ratio of about 1:1:2, and the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

In some embodiments, the multispecific antibody comprises two antigen binding domains that bind CD40 and one antigen binding domain that binds FAP, wherein the multispecific antibody comprises a first polypeptide, a second polypeptide, and a third polypeptide at a ratio of about 1:1:2, and the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

In some embodiments, the multispecific antibody comprises two antigen binding domains that bind CD40 and one antigen binding domain that binds FAP, wherein the multispecific antibody comprises a first polypeptide, a second polypeptide, and a third polypeptide at a ratio of about 1:1:2, and the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

### IV. EXEMPLARY ANTIBODY VARIANTS, FRAGMENTS, AND CONSTANT REGIONS

In many embodiments, an antibody specifically binding to CD40 and/or FAP (including a multispecific antibody) may further incorporate any of the features, singly or in combination, as described in the sections that follow.

### A. Antibody Fragments

In certain embodiments, an antigen binding domain provided herein is an antibody fragment. In some embodiments, the antigen binding domain is an antibody fragment selected from a Fab, Fab′, F(ab′)₂, Fd, Fv, single-chain Fv (scFv) or disulfide-linked Fv (sdFv), and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,187,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### B. Human Antibodies

In certain embodiments, an antibody provided herein is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90:2551-55 (1993); Jakobovits et al., Nature 362:255-8 (1993); Lonberg et al., Nature 368:856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include selecting human antibodies from phage display libraries. See, e.g., Hoogenboom et al., J. Mol. Biol. 227:381-8 (1992); Marks et al., J. Mol. Biol. 222:581-97 (1991); and PCT Publication No. WO 99/10494.

### C. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. In some embodiments, an antibody provided herein is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carte r et al. Pro c. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the humanized antibodies may comprise a human IgG1, IgG2, IgG3, or IgG4 heavy chain constant region.

### D. Glycosylation and Pegylation Variants

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally, or alternatively, an antibody can be made that has an altered type of glycosylation, such as a hypofucosylated antibody having reduced amounts of fucosyl residues or an antibody having increased bisecting GlcNac structures. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn (297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta (1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

### E. Constant Regions

In some embodiments, an antibody is a full-length antibody. In some embodiments, a multispecific antibody comprises a full-length antibody. In some embodiments, an antibody described herein comprises one or more human constant regions. In some embodiments, the human heavy chain constant region is of an isotype selected from IgA, IgG, and IgD. In some embodiments, the human light chain constant region is of an isotype selected from k and 2. In some embodiments, the antibody is an IgG antibody. In some embodiments, the antibody is an IgG antibody lacking a C-terminal lysine in the heavy chain constant region. In some embodiments, an antibody described herein comprises a human IgG constant region, such as an IgG1, IgG2, IgG3, or IgG4. In some embodiments, the antibody is an IgG1 antibody, an IgG2 antibody, an IgG3 antibody, or an IgG4 antibody. In some embodiments, an antibody described herein comprises a human IgG1 heavy chain constant region. In some embodiments, an antibody described herein comprises a human IgG1 constant region and a human k light chain.

In some embodiments, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain and/or a cleaved variant of the full-length heavy chain. In some embodiments, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine.

In some embodiments, the first and/or second Fc polypeptide chain comprises a post-translational modification. In some embodiments, the first and/or second Fc polypeptide chain comprises a post-translational modification that is post-translational cleavage of one or more amino acids from the C-terminus of the heavy chain. In some embodiments, the Fc polypeptide chain comprising the amino acid sequence set forth in SEQ ID NO: 51 undergoes a post-translational cleavage to become the amino acid sequence set forth in SEQ ID NO: 55. In some embodiments, the Fc polypeptide chain comprising the amino acid sequence set forth in SEQ ID NO: 52 undergoes a post-translational cleavage to become the amino acid sequence set forth in SEQ ID NO: 56.

In some embodiments, the first Fc polypeptide chain comprises an amino acid sequence at least 85%, at least 90%, or at least 95% identical to the amino acid sequence of SEQ ID NO: 55. In some embodiments, the first Fc polypeptide chain comprises the amino acid sequence of SEQ ID NO: 55. In some embodiments, the first Fc polypeptide chain comprises an amino acid sequence at least 85%, at least 90%, or at least 95% identical to the amino acid sequence of SEQ ID NO: 56. In some embodiments, the first Fc polypeptide chain comprises the amino acid sequence of SEQ ID NO: 56.

In some embodiments, the second Fc polypeptide chain comprises an amino acid sequence at least 85%, at least 90%, or at least 95% identical to the amino acid sequence of SEQ ID NO: 55. In some embodiments, the second Fc polypeptide chain comprises the amino acid sequence of SEQ ID NO: 55. In some embodiments, the second Fc polypeptide chain comprises an amino acid sequence at least 85%, at least 90%, or at least 95% identical to the amino acid sequence of SEQ ID NO: 56. In some embodiments, the second Fc polypeptide chain comprises the amino acid sequence of SEQ ID NO: 56.

In some embodiments, the first and/or second polypeptide comprising a heavy chain comprises a post-translational modification. In some embodiments, the first and/or second polypeptide comprises a post-translational modification that is post-translational cleavage of one or more amino acids from the C-terminus of the heavy chain. In some embodiments, the first and/or second polypeptide comprising the amino acid sequence set forth in SEQ ID NO: 51 undergoes a post-translational cleavage to become the amino acid sequence set forth in SEQ ID NO: 55. In some embodiments, the first and/or second polypeptide comprising the amino acid sequence set forth in SEQ ID NO: 52 undergoes a post-translational cleavage to become the amino acid sequence set forth in SEQ ID NO: 56.

In some embodiments, the first polypeptide of the multispecific antibody comprises a post-translational modification. In some embodiments, the first polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 58. In some embodiments, the second polypeptide comprises the amino acid sequence of SEQ ID NO: 58. In some embodiments, the multispecific antibody comprises the second polypeptide sequence of SEQ ID NO: 58.

In some embodiments, the second polypeptide of the multispecific antibody comprises a post-translational modification. In some embodiments, the second polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 43. In some embodiments, the second polypeptide comprises the amino acid sequence of SEQ ID NO: 43. In some embodiments, the multispecific antibody comprises the second polypeptide sequence of SEQ ID NO: 43.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, an antibody comprising a human IgG1 heavy chain or a human IgG3 heavy chain is selected.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. Sec, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibodies with certain improved properties. For example, an antibody may be afucosylated, for example, by mutating residues such as Asn297 that are normally glycosylated with fucose-containing glycosylations, or through other means.

Antibodies are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody.

The in vivo or serum half-life of human FcRn high affinity binding polypeptides can be assayed, e.g., in transgenic mice, in humans, or in non-human primates to which the polypeptides with a variant Fc region are administered. See also, e.g., Petkova et al. International Immunology 18 (12): 1759-1769 (2006).

In certain embodiments, the Fc region is altered by replacing at least one amino acid residue with a different amino acid residue to alter the effector function(s) of the antibody. For example, one or more amino acids selected from amino acid residues 234, 235, 236, 237, 265, 297, 318, 320, 322, 330, and/or 331 (EU numbering) can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some examples, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogic et al.

In some embodiments, a multispecific antibody comprises one or more mutations in the heavy chain constant region that reduce or eliminate binding to FcγR. In some embodiments, the multispecific antibody does not bind FcγR or has reduced binding to FcγR compared to a multispecific antibody having the same antigen binding domains but a wild-type constant region of the same isotype. In some examples, the Fc region can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (EU numbering). Exemplary substitutions include 234A, 235A, 236A, 239D, 239E, 265S, 268D, 267E, 268E, 268F, 324T, 332D, and 332E. Exemplary variants include 234A/235A/265S, 239D/332E, 236A/332E, 236A/239D/332E, 268F/324T, 267E/268F, 267E/324T, and 267E/268F7324T (EU numbering). Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 265, 267, 269, 325, 328, 330, and/or 331 (e.g., 330 and 331), wherein numbering is according to the EU index. Exemplary substitutions include but are not limited to 234A, 235A, 235E, 236R, 237A, 265S, 267R, 269R, 325L, 328R, 330S, and 331S (e.g., 330S, and 331S), wherein numbering is according to the EU index. An Fc variant can comprise 234A/235A/265S. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691. For example, a human IgG1 constant region may comprise L234A, L235E, and G237A substitutions. In some embodiments, a human IgG1 constant region may comprise a P238K substitution. In some embodiments, a human IgG1 constant region may comprise L234A, L235E, G237A, A330S, and P331S substitutions. In some embodiments, a human IgG1 constant region may comprise L234A, L235A, and D265S substitutions. (All numbering under the EU index.)

Fc variants that enhance affinity for an inhibitory receptor FcγRIIb can also be used. Such variants can provide an Fc fusion protein with immunomodulatory activities related to FcγRIIb cells, including for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, 330, 331, and 332, according to the EU index. Exemplary substitutions for enhancing FcγRllb affinity include but are not limited to 234A, 234D, 234E, 234F, 234W, 235D, 235E, 235F, 235R, 235Y, 236D, 236N, 237A, 237D, 237N, 239D, 239E, 266M, 267D, 267E, 268D, 268E, 327D, 327E, 328F, 328W, 328Y, 330S, 331S, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F. (All numbering under the EU index.)

Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691. Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Patent Publication No. WO 00/42072.

Optionally, the Fc region can comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

The affinities and binding properties of an Fc region for its ligand can be determined by a variety of in vitro assay methods (biochemical or immunological based assays) known in the art including but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this can be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J. Biol. Chem. 279 (8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276 (9): 6591-6604), 252F, 252T, 252Y, 252W, 254T, 256S, 256R, 256Q, 256E, 256D, 256T, 309P, 311 S, 433R, 433S, 4331, 433P, 433Q, 434H, 434F, 434Y, 252Y/254T/256E, 433K/434F/436H, 308T/309P/311S (Dall'Acqua et al. Journal of Immunology, 2002, 169:5171-5180, Dal'Acqua et al., 2006, Journal of Biological Chemistry 281:23514-23524). Other modifications for modulating FcRn binding are described in Young et al., 2010, J Immunol, 182:7663-7671.

In certain embodiments, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus, a hybrid variant IgG antibody can be constructed that comprises one or more substitutions, e.g., 274Q, 276K, 300F, 339T, 356E, 358M, 384S, 392N, 397M, 422I, 435R, and 436F. In some embodiments described herein, an IgG1/IgG2 hybrid variant can be constructed by substituting IgG2 positions in the CH2 and/or CH3 region with amino acids from IgG1 at positions where the two isotypes differ. Thus, a hybrid variant IgG antibody can be constructed that comprises one or more substitutions, e.g., one or more of the following amino acid substitutions: 233E, 234L, 235L, +236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/1332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/1332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/1332E, S239D/1332E/A330L, L235V/F243L/R292P/Y300L/P396L, and M428L/N434S.

In certain embodiments, an Fc is chosen that has essentially no effector function, i.e., it has reduced binding to FcγRs and reduced complement fixation. An exemplary Fc, e.g., IgG1 Fc, that is effectorless comprises the following three mutations: L234A, L235A, and D265S.

When using an IgG4 constant domain, it can include the substitution S228P, which mimics the hinge sequence in IgG1 and thereby stabilizes IgG4 molecules. Fc modifications described in WO 2017/087678 or WO2016081746 may also be used.

In certain embodiments, the glycosylation of an antibody is modified. For example, an aglycoslated antibody can be made (i.e., the antibody lacks glycosylation). Glycosylation of the constant region on N297 can be prevented by mutating the N297 residue to another residue, e.g., N297A, and/or by mutating an adjacent amino acid, e.g., 298 to thereby reduce glycosylation on N297.

### V. PHARMACEUTICAL COMPOSITIONS

Pharmaceutical compositions comprising an antibody described herein are provided. In some embodiments, the pharmaceutical composition comprises the antibody and a pharmaceutically acceptable carrier.

In various embodiments, compositions comprising an antibody provided herein are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

In some embodiments, the pharmaceutically acceptable carrier may be a liquid or solid filler, diluent, excipient, solvent, or encapsulating material, or a combination thereof. A pharmaceutically acceptable carrier includes, but is not limited to, a buffer, excipient, stabilizer, or preservative.

### VI. NUCLEIC ACID MOLECULES ENCODING MULTISPECIFIC ANTIBODIES

Nucleic acid molecules comprising polynucleotides that encode one or more chains of an antibody (such as a multispecific antibody) described herein are provided.

In some embodiments, a nucleic acid molecule comprises a polynucleotide that encodes a first polypeptide of a multispecific antibody that bind to CD40 and FAP provided herein. In some embodiments, a nucleic acid molecule comprises both a polynucleotide that encodes a first polypeptide and a polynucleotide that encodes a second polypeptide, of a multispecific antibody. In some embodiments, a first nucleic acid molecule comprises a first polynucleotide that encodes a first polypeptide and a second nucleic acid molecule comprises a second polynucleotide that encodes a second polypeptide. In some embodiments, a nucleic acid molecule comprises a polynucleotide that encodes a first polypeptide, a second polypeptide, and a third polypeptide of the multispecific antibody provided herein. In some embodiments, a first nucleic acid molecule comprises a first polynucleotide that encodes a first polypeptide, a second nucleic acid molecule comprises a second polynucleotide that encodes a second polypeptide, and a third nucleic acid molecule comprises a third polynucleotide that encodes a third polypeptide. In some embodiments, the first polynucleotide encodes a first polypeptide comprising the amino acid sequence of SEQ ID NO: 34, the second polynucleotide encodes a second polypeptide comprising polypeptide comprises the amino acid sequence of SEQ ID NO: 15, and the third polynucleotide encodes a third polypeptide comprising the amino acid sequence of SEQ ID NO: 16.

In some embodiments, the first polypeptide, second polypeptide, and third polypeptide are expressed from one nucleic acid molecule, or two separate nucleic acid molecules or from three separate nucleic acid molecules, as three separate polypeptides. In some embodiments, such as when an antibody is an scFv, a single polynucleotide encodes a single polypeptide comprising both a heavy chain and a light chain linked together.

In some embodiments, a polynucleotide encoding a first polypeptide, second polypeptide, and third polypeptide of a multispecific antibody that binds to CD40 and FAP comprises a nucleotide sequence that encodes a leader sequence, which, when translated, is located at the N terminus of an antibody heavy chain or light chain. The leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell. In some embodiments, an isolated nucleic acid molecule is an expression vector that is suitable for expression in a selected host cell.

### VII. MULTISPECIFIC ANTIBODY EXPRESSION AND PRODUCTION

### A. Vectors

In some embodiments, vectors comprising polynucleotides that encode one or more heavy chains and/or light chains of an antibody described herein are provided. In some embodiments, vectors comprising polynucleotides that encode one or more polypeptides of a multispecific antibody described herein are provided. In some embodiments, provided herein is a vector comprising a nucleic acid encoding any one of the multispecific antibodies that bind to CD40 and FAP described herein. Vectors comprising polynucleotides that encode one or more polypeptides of a multispecific antibody are also provided. Such vectors include, but are not limited to, DNA vectors, RNA vectors (e.g., mRNA and circular RNA, self-amplifying RNA vectors, etc.), phage vectors, viral vectors (e.g., pox virus vectors, vaccinia virus vectors, adenovirus vectors, modified vaccinia virus Ankara (MVA) vectors, etc.), retroviral vectors, etc. In some embodiments, a vector comprises a first polynucleotide sequence encoding a first polypeptide, a second polynucleotide sequence encoding a second polypeptide, and a third polynucleotide sequence encoding a third polypeptide of a multispecific antibody provided herein. In some embodiments, the first polypeptide, second polypeptide, and third polypeptide are expressed from the vector as three separate polypeptides. In some embodiments, the first polypeptide, second polypeptide, and third polypeptide are expressed from three separate vectors as three separate polypeptides. In some embodiments, the first polynucleotide encodes a first polypeptide comprising the amino acid sequence of SEQ ID NO: 34, the second polynucleotide encodes a second polypeptide comprising polypeptide comprises the amino acid sequence of SEQ ID NO: 15, and the third polynucleotide encodes a third polypeptide comprising the amino acid sequence of SEQ ID NO: 16.

In some embodiments, the first polypeptide, second polypeptide, and third polypeptide of the multispecific antibody described herein are expressed from three separate vectors as three separate polypeptides. In some embodiments, a first vector comprises a first polynucleotide sequence encoding a first polypeptide, a second vector comprises a second polynucleotide sequence encoding a second polypeptide, and a third vector comprises a third polynucleotide sequence encoding a third polypeptide. In some embodiments, the first vector, the second vector, and the third vector are transfected into host cells in similar amounts (such as similar molar amounts or similar mass amounts). In some embodiments, the first vector, second vector, and third vector are transfected into host cells at a mole- or mass-ratio of between 5:1 and 1:5 when comparing any two of the first vector, the second vector, and the third vector.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In some embodiments, a vector is chosen for in vivo expression multispecific antibodies comprising two or more polypeptides in animals, including humans. In some such embodiments, expression of the polypeptide is under the control of a promoter that functions in a tissue-specific manner. For example, liver-specific promoters are described, e.g., in PCT Publication No. WO 2006/076288.

### B. Host Cells

In various embodiments, an antibody (such as a multispecific antibody that bind to CD40 and FAP) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. In some embodiments, provided herein are host cells comprising an isolated nucleic acid encoding any one of the antibodies described herein. In some embodiments, provided herein are host cells comprising a vector comprising a nucleic acid encoding any one of the antibodies described herein.

In some embodiments, provided herein is a host cell that produces an antibody (such as a multispecific antibody) described herein. In some embodiments, provided herein are host cells comprising a first vector comprising a first nucleic acid encoding a first polypeptide of the multispecific antibody described herein, a second vector comprising a second nucleic acid encoding a second polypeptide of the multispecific antibody described herein, and a third vector comprising a third nucleic acid encoding a third polypeptide of the multispecific antibody described herein. In some embodiments, provided herein is a host cell that produces any one of the multispecific antibodies that bind to CD40 and FAP described herein. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, an antibody may be expressed in yeast. Sec, e.g., U.S. Publication No. US 2006/0270045 A1. In some embodiments, a particular eukaryotic host cell is selected based on its ability to make desired post-translational modifications to the antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Provided herein is a host cell comprising a first polynucleotide sequence that encodes the first polypeptide, a second polynucleotide sequence that encodes the second polypeptide, and a third nucleic acid sequence that encodes the third polypeptide, of the multispecific antibody described herein.

In some embodiments, provided herein is a method for producing an antibody described herein, comprising culturing a host cell described herein under conditions suitable for expression of the antibody. In some embodiments, the method further comprises recovering the antibody produced by the host cell. In some embodiments, the method further comprises isolating the antibody.

### C. Purification of Antibodies

The antibodies (such as multispecific antibodies) described herein may be purified by any suitable method. Such methods include, but are not limited to, the use of affinity matrices or hydrophobic interaction chromatography or size exclusion chromatography. Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). Multispecific antibodies may also be made by engineering electrostatic steering effects for making antibody Fc-heterodimeric molecules (WO 2009/089004A1, Escobar-Cabrera et al., Antibodies (Basel), 6(2): 7 (2017), and WO 2013/166594); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 18(5): 1547-1553(1992)); using “diabod” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147:60 (1991).

In some embodiments, an antibody is produced in a cell-free system. Nonlimiting exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

### VIII. THERAPEUTIC COMPOSITIONS AND METHODS

### A. Methods of Treating Diseases

Provided herein is a method of treating and/or preventing cancer, which comprises administering to a patient an antibody (e.g., a multispecific antibody) or a pharmaceutical composition thereof provided herein. The antibodies provided herein are useful for targeting an antigen overexpressed in cancer (e.g., FAP) and stimulating immune cell activity. In the presence of FAP-expressing cells, the antibodies provided herein are CD40 agonists, resulting in dendritic cell (DC), macrophage, and/or B cell activation for treating a patient having cancer.

In another embodiment, provided herein is method of managing cancer, which comprises administering to a patient an antibody or a pharmaceutical composition thereof provided herein.

Also provided herein are methods of treating patients who have been previously treated for cancer but are non-responsive to standard therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In certain embodiments, the cancer is a solid tumor. In certain embodiments, the solid tumor is metastatic. In certain embodiments, the solid tumor is gastric cancer, pancreatic ductal adenocarcinoma (PDAC), non-small cell lung cancer (NSCLC), colorectal cancer (CRC), or squamous cell carcinoma of head and neck (SCCHN).

Also provided herein is a method of treating cancer comprising administering to a subject in need thereof any one of the multispecific antibodies or any one of the pharmaceutical compositions described herein. In some embodiments, the cancer is a solid cancer. In some embodiments, solid cancer is gastric cancer or pancreatic cancer. In some embodiments, the pancreatic cancer is pancreatic ductal adenocarcinoma.

In certain embodiments, the patient to be treated with one of the methods provided herein has not been treated with anticancer therapy prior to the administration of an antibody provided herein. In certain embodiments, the patient to be treated with one of the methods provided herein has been treated with anticancer therapy prior to the administration of an antibody provided herein. In certain embodiments, the patient to be treated with one of the methods provided herein has developed drug resistance to the anticancer therapy.

The methods provided herein encompass treating a patient regardless of patient's age, although some diseases or disorders are more common in certain age groups.

In certain embodiments, provided herein is a method of treating cancer comprising administering to a subject in need thereof the multispecific antibody, the antibody or antigen-binding fragment thereof, or the pharmaceutical composition as described herein (e.g., as in Sections III, IV, and V).

In certain embodiments, provided herein is a method of treating cancer comprising administering to a subject in need thereof a multispecific antibody, wherein the multispecific antibody comprises two antigen binding domains that bind CD40 and one antigen binding domain that binds FAP. In some embodiments, the multispecific antibody comprises a first polypeptide, a second polypeptide, and a third polypeptide at a ratio of 1:1:2. In some embodiments, the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

In some embodiments, the cancer is a solid tumor. In some embodiments, the cancer is gastric cancer or pancreatic cancer. In some embodiments, the cancer is gastric cancer. In some embodiments, the pancreatic cancer is pancreatic ductal adenocarcinoma.

In some embodiments, the cancer is selected from non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN). In some embodiments, the cancer is NSCLC. In some embodiments, the cancer is MSS CRC. In some embodiments, the cancer is PDAC. In some embodiments, the cancer is G/GEJC. In some embodiments, the cancer is SCCHN.

In some embodiments, the cancer is advanced unresectable cancer. In some embodiments, the cancer is metastatic cancer. In some embodiments, the cancer is recurrent cancer. In some embodiments, the cancer is locally advanced unresectable, metastatic, or recurrent malignant tumor selected from NSCLC, MSS CRC, PDAC, G/GEJC, and SCCHN.

In some embodiments, the method of treating any one of the cancers described herein comprises administering to a subject between about 10 mg to about 1000 mg of the multispecific antibody. In some embodiments, the method comprises administering to a subject between about 10 mg to about 1000 mg, about 15 mg to about 950 mg, about 20 mg to about 900 mg, about 25 mg to about 850 mg, about 30 mg to about 800 mg, about 35 mg to about 750 mg, about 40 mg to about 700 mg, about 45 mg to about 650 mg, about 50 mg to about 600 mg, about 55 mg to about 550 mg, about 60 mg to about 500 mg, about 65 mg to about 450 mg, about 70 mg to about 400 mg, about 75 mg to about 350 mg, about 80 mg to about 300 mg, about 85 mg to about 250 mg, about 90 mg to about 200 mg, or about 95 mg to about 150 mg of the multispecific antibody. In some embodiments, the method comprises administering to a subject about 10, 15, 20, 25, 30, 35, 40, 45, 50, 55, 60, 65, 70, 75, 80, 85, 90, 95, or 100 mg of the multispecific antibody. In some embodiments, the method comprises administering to a subject 10, 15, 20, 25, 30, 35, 40, 45, 50, 55, 60, 65, 70, 75, 80, 85, 90, 95, or 100 mg of the multispecific antibody. In some embodiments, the method comprises administering to a subject about 10, 11, 12, 13, 14, 15, 16, 17, 18, 19, 20, 21, 22, 23, 24, 25, 26, 27, 28, 29, 30, 31, 32, 33, 34, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg of the multispecific antibody. In some embodiments, the method comprises administering to a subject about 36, 37, 38, or 39 mg of the multispecific antibody. In some embodiments, the method comprises administering to a subject 10, 11, 12, 13, 14, 15, 16, 17, 18, 19, 20, 21, 22, 23, 24, 25, 26, 27, 28, 29, 30, 31, 32, 33, 34, 35, 40, 41, 42, 43, 44, 45, 46, 47, 48, 49, 50, 51, 52, 53, 54, 55, 56, 57, 58, 59, 60, 61, 62, 63, 64, 65, 66, 67,68, 69, 70,71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg of the multispecific antibody. In some embodiments, the method comprises administering to a subject 36, 37, 38, or 39 mg of the multispecific antibody. In some embodiments, the method comprises administering to a subject about 100, 150, 200, 250, 300, 350, 400, 450, 500, 550, 600, 650, 700, 750, 800, 850, 900, 950, or 1000 mg of the multispecific antibody. In some embodiments, the method comprises administering to a subject 100, 150, 200, 250, 300, 350, 400, 450, 500, 550, 600, 650, 700, 750, 800, 850, 900, 950, or 1000 mg of the multispecific antibody.

In some embodiments, the method comprises administering to a subject about 10 mg of the multispecific antibody. In some embodiments, the method comprises administering to a subject 10 mg of the multispecific antibody. In some embodiments, the method comprises administering to a subject about 30 mg of the multispecific antibody. In some embodiments, the method comprises administering to a subject 30 mg of the multispecific antibody. In some embodiments, the method comprises administering to a subject about 90 mg of the multispecific antibody. In some embodiments, the method comprises administering to a subject 90 mg of the multispecific antibody. In some embodiments, the method comprises administering to a subject about 250 mg of the multispecific antibody. In some embodiments, the method comprises administering to a subject 250 mg of the multispecific antibody. In some embodiments, the method comprises administering to a subject about 500 mg of the multispecific antibody. In some embodiments, the method comprises administering to a subject 500 mg of the multispecific antibody. In some embodiments, the method comprises administering to a subject about 1000 mg of the multispecific antibody. In some embodiments, the method comprises administering to a subject 1000 mg of the multispecific antibody.

In some embodiments, the multispecific antibody is administered in a cycling regimen. In some embodiments, the cycling regimen comprises one or more 21-day cycles. In some embodiments, the cycling regimen comprises one or more 28-day cycles. In some embodiments, each cycle of the cycling regimen is the same. In each cycling regimen, Day 1 is the first day of each cycle. In some embodiments, the multispecific antibody is administered on Day 1 of each 21-day cycle. In some embodiments, the multispecific antibody is administered on Day 1 of each 28-day cycle. In some embodiments, the multispecific antibody is administered on Days 1 and 15 of each 28-day cycle.

The multispecific antibody, in some instances, can be administered at any described dose according to a schedule described herein. In some embodiments, the multispecific antibody is administered to the subject about once every 1, 2, 3, 4, 5, 6, 7, 8, 9, or 10 weeks. In some embodiments, the multispecific antibody is administered to the subject about once every week, about once every two weeks, about once every three weeks, about once every four weeks, about once every 5 weeks, about once every 6 weeks, about once every 7 weeks, about once every 8 weeks, about once every 9 weeks, or about once every 10 weeks. In some embodiments, the multispecific antibody is administered to the subject about once every week, about once every two weeks, about once every three weeks, or about once every four weeks. In some embodiments, the multispecific antibody is administered to the subject about once every two weeks.

The method provided herein, in some instances, further comprises administering at least one PD-1 therapy. In some embodiments, the at least one PD-1 therapy is a PD-1 therapy. In some embodiments, the PD-1 therapy is an antibody or antigen-binding fragment thereof that binds to PD-1, or an antibody or antigen-binding fragment thereof that binds to PD-L1. In some embodiments, the PD-1 therapy is a small molecule targeting PD-1 or PD-L1.

In some embodiments, the multispecific antibody and the PD-1 therapy are administered separately. In some embodiments, the multispecific antibody and the PD-1 therapy are administered sequentially. In some embodiments, the multispecific antibody is administered after the PD-1 therapy. In some embodiments, the PD-1 therapy is administered after the multispecific antibody. In some embodiments, the multispecific antibody and the PD-1 therapy are co-administered, wherein the multispecific antibody is in a first bag and the PD-1 therapy is in a second bag, and wherein the multispecific antibody and the PD-1 therapy are administered simultaneously.

In some embodiments, the PD-1 therapy is administered to the subject by intravenous administration. In some embodiments, the PD-1 therapy is administered to the subject by subcutaneous administration. In some embodiments, the PD-1 therapy is administered to the subject by any other standard means known in the art.

In some embodiments, the PD-1 therapy is administered to the subject in a cycling regimen, such as any cycling regimen described herein. In some embodiments, the PD-1 therapy is administered on Day 1 of each 21-day cycle. In some embodiments, the PD-1 therapy is administered on Day 1 of each 28-day cycle. In some embodiments, the PD-1 therapy is administered on Days 1 and 15 of each 28-day cycle.

The PD-1 therapy, in some instances, is selected from nivolumab, pembrolizumab, cemiplimab, dostarlimab, retifanlimab, toripalimab, atezolizumab, avelumab, and durvalumab.

In some embodiments, the PD-1 therapy is nivolumab. In some embodiments, the PD-1 therapy is pembrolizumab. In some embodiments, the PD-1 therapy is cemiplimab. In some embodiments, the PD-1 therapy is dostarlimab. In some embodiments, the PD-1 therapy is retifanlimab. In some embodiments, the PD-1 therapy is toripalimab. In some embodiments, the PD-1 therapy is atezolizumab. In some embodiments, the PD-1 therapy is avelumab. In some embodiments, the PD-1 therapy is durvalumab.

In some embodiments, the PD-1 therapy is nivolumab, and nivolumab is administered to the subject about once every two weeks at a dose of 240 mg, about once every three weeks at a dose of 360 mg, or about once every four weeks at a dose of 480 mg. In some embodiments, nivolumab is administered to the subject once every two weeks at a dose of or about 320 mg to a dose to or to about 600 mg, inclusive. In some embodiments, nivolumab is administered to the subject about once every two weeks at a dose of 240 mg. In some embodiments, nivolumab is administered to the subject about once every two weeks at a dose of or about 600 mg. In some embodiments, nivolumab is administered to the subject about once every three weeks at a dose of or about 360 mg to a dose to or to about 1200 mg, inclusive. In some embodiments, nivolumab is administered to the subject about once every three weeks at a dose of 360 mg. In some embodiments, nivolumab is administered to the subject about once every three weeks at a dose of or about 720 mg. In some embodiments, nivolumab is administered to the subject about once every three weeks at a dose of or about 900 mg. In some embodiments, nivolumab is administered to the subject about once every three weeks at a dose of or about 960 mg. In some embodiments, nivolumab is administered to the subject about once every three weeks at a dose of or about 1200 mg. In some embodiments, nivolumab is administered to the subject about once every four weeks at a dose of or about 480 mg to or to about 1200 mg, inclusive. In some embodiments, nivolumab is administered to the subject about once every four weeks at a dose of 480 mg. In some embodiments, nivolumab is administered to the subject about once every four weeks at a dose of or about 720 mg. In some embodiments, nivolumab is administered to the subject about once every four weeks at a dose of or about 960 mg. In some embodiments, nivolumab is administered to the subject about once every four weeks at a dose of or about 1200 mg.

In some embodiments, nivolumab is administered intravenously to the subject about once every four weeks at a dose or of about 480 mg.

In some embodiments, nivolumab is administered subcutaneously to the subject once every two to four weeks at a dose of or about 600 mg to or to about 1200 mg, inclusive. In some embodiments, nivolumab is administered subcutaneously to the subject about once every two weeks (Q2W) at a dose of or about 600 mg. In some embodiments, nivolumab is administered subcutaneously to the subject about once every three weeks (Q3W) at a dose of or about 720 mg, of or about 900 mg, of or about 960 mg, or of or about 1200 mg. In some embodiments, nivolumab is administered subcutaneously to the subject about once every four weeks (Q4W) at a dose of or about 720 mg, of or about 960 mg, or of or about 1200 mg. In some embodiments, nivolumab is administered subcutaneously to the subject about once every four weeks (Q4W) at a dose of or about 1200 mg.

In some of any such embodiments, the nivolumab administered subcutaneously is co-formulated with hyaluronidase. Co-formulation of nivolumab with hyaluronidase allows administration of larger volumes with fewer injections subcutaneously than without hyaluronidase, as described in Albiges L et al., Ann Oncol. 2025; 36 (1): 99-107, which is incorporated by reference in its entirety. In some embodiments, the hyaluronidase is recombinant human hyaluronidase (rHuPH20; Halozyme Therapeutics, Inc.), which is a recombinant form of human PH20 that is a 447 amino acid sequence devoid of the GPI anchor attachment motif. Sec e.g., Bookbinder et al. (2006) Journal of Controlled Release, 114:230-241; described in Locke et al. (2019) Drug Delivery, 26:98-106, which are incorporated by reference in their entirety. In some embodiments, nivolumab is co-formulated with hyaluronidase at a dose of or about 10,000 units to 20,000 units, inclusive. In some embodiments, nivolumab is co-formulated with hyaluronidase at a dose of or about 10,000 units; of or about 12,000 units; of or about 15,000 units; of or about 16,000 units; or of or about 20,000 units.

In some embodiments, nivolumab co-formulated with hyaluronidase is administered subcutaneously to the subject once every two to four weeks at a dose of or about 600 mg to or to about 1200 mg nivolumab, inclusive, and of or about 10,000 units to of or about 20,000 units hyaluronidase, inclusive.

In some embodiments, nivolumab co-formulated with hyaluronidase is administered subcutaneously to the subject once every two weeks at a dose of or about 720 mg nivolumab and of or about 20,000 units hyaluronidase. In some embodiments, nivolumab co-formulated with hyaluronidase is administered subcutaneously to the subject once every two weeks at a dose of or about 960 mg nivolumab and of or about 20,000 units hyaluronidase. In some embodiments, nivolumab co-formulated with hyaluronidase is administered subcutaneously to the subject once every two weeks at a dose of or about 1200 mg nivolumab and of or about 20,000 units hyaluronidase. In some embodiments, nivolumab co-formulated with hyaluronidase is administered subcutaneously to the subject once every two weeks at a dose of or about 720 mg nivolumab and of or about 12,000 units hyaluronidase. In some embodiments, nivolumab co-formulated with hyaluronidase is administered subcutaneously to the subject once every two weeks at a dose of or about 960 mg nivolumab and of or about 12,000 units hyaluronidase. In some embodiments, nivolumab co-formulated with hyaluronidase is administered subcutaneously to the subject once every two weeks at a dose of or about 1200 mg nivolumab and of or about 12,000 units hyaluronidase.

In some embodiments, nivolumab co-formulated with hyaluronidase is administered subcutaneously to the subject once every two weeks at a dose of or about 600 mg nivolumab and of or about 10,000 units hyaluronidase.

In some embodiments, nivolumab co-formulated with hyaluronidase is administered subcutaneously to the subject once every three weeks at a dose of or about 720 mg nivolumab and of or about 20,000 units hyaluronidase. In some embodiments, nivolumab co-formulated with hyaluronidase is administered subcutaneously to the subject once every three weeks at a dose of or about 960 mg nivolumab and of or about 20,000 units hyaluronidase. In some embodiments, nivolumab co-formulated with hyaluronidase is administered subcutaneously to the subject once every three weeks at a dose of or about 1200 mg nivolumab and of or about 20,000 units hyaluronidase. In some embodiments, nivolumab co-formulated with hyaluronidase is administered subcutaneously to the subject once every three weeks at a dose of or about 720 mg nivolumab and of or about 12,000 units hyaluronidase. In some embodiments, nivolumab co-formulated with hyaluronidase is administered subcutaneously to the subject once every three weeks at a dose of or about 960 mg nivolumab and of or about 12,000 units hyaluronidase. In some embodiments, nivolumab co-formulated with hyaluronidase is administered subcutaneously to the subject once every three weeks at a dose of or about 1200 mg nivolumab and of or about 12,000 units hyaluronidase.

In some embodiments, nivolumab co-formulated with hyaluronidase is administered subcutaneously to the subject once every three weeks at a dose of or about 900 mg nivolumab and of or about 15,000 units hyaluronidase.

In some embodiments, nivolumab co-formulated with hyaluronidase is administered subcutaneously to the subject once every four weeks at a dose of or about 720 mg nivolumab and of or about 20,000 units hyaluronidase. In some embodiments, nivolumab co-formulated with hyaluronidase is administered subcutaneously to the subject once every four weeks at a dose of or about 960 mg nivolumab and of or about 20,000 units hyaluronidase. In some embodiments, nivolumab co-formulated with hyaluronidase is administered subcutaneously to the subject once every four weeks at a dose of or about 1200 mg nivolumab and of or about 20,000 units hyaluronidase. In some embodiments, nivolumab co-formulated with hyaluronidase is administered subcutaneously to the subject once every four weeks at a dose of or about 720 mg nivolumab and of or about 12,000 units hyaluronidase. In some embodiments, nivolumab co-formulated with hyaluronidase is administered subcutaneously to the subject once every four weeks at a dose of or about 960 mg nivolumab and of or about 12,000 units hyaluronidase. In some embodiments, nivolumab co-formulated with hyaluronidase is administered subcutaneously to the subject once every four weeks at a dose of or about 1200 mg nivolumab and of or about 12,000 units hyaluronidase.

In some embodiments, nivolumab co-formulated with hyaluronidase is administered subcutaneously to the subject once every four weeks at a dose of or about 1200 mg nivolumab and of or about 20,000 units hyaluronidase.

In some embodiments, the PD-1 therapy is pembrolizumab, and pembrolizumab is administered to the subject about once every three weeks at a dose of 200 mg or about once every six weeks at a dose of 400 mg. In some embodiments, pembrolizumab is administered to the subject about once every three weeks at a dose of 200 mg. In some embodiments, pembrolizumab is administered to the subject about once every six weeks at a dose of 400 mg.

In some embodiments, the PD-1 therapy is cemiplimab, and cemiplimab is administered to the subject about once every three weeks at a dose of 350 mg.

In some embodiments, the PD-1 therapy is dostarlimab, and dostarlimab is administered to the subject about once every three weeks at a dose of 500 mg for dose 1 through dose 4 and about once every six weeks at a dose of 1000 mg for dose 5 onwards.

In some embodiments, the PD-1 therapy is retifanlimab, and retifanlimab is administered to the subject about once every four weeks at a dose of 500 mg.

In some embodiments, the PD-1 therapy is toripalimab, and toripalimab is administered to the subject about once every two weeks at a dose of 3 mg/kg.

In some embodiments, the PD-1 therapy is atezolizumab, and atezolizumab is administered to the subject about once every two weeks at a dose of 840 mg, once every three weeks at a dose of 1200 mg, or once every four weeks at a dose of 1680 mg. In some embodiments, atezolizumab is administered to the subject about once every two weeks at a dose of 840 mg. In some embodiments, atezolizumab is administered to the subject about once every three weeks at a dose of 1200 mg. In some embodiments, atezolizumab is administered to the subject about once every four weeks at a dose of 1680 mg.

In some embodiments, the PD-1 therapy is avelumab, and avelumab is administered to the subject about once every two weeks at a dose of 800 mg.

In some embodiments, the PD-1 therapy is durvalumab, and durvalumab is administered to the subject about once every two weeks at a dose of 10 mg/kg.

In some embodiments, the method further comprises administering a chemotherapy.

In some embodiments, the chemotherapy is CAFOX. In some embodiments, the chemotherapy comprises capecitabine and oxaliplatin. In some embodiments, the chemotherapy is administered in a cycling regimen, such as any described herein. In some embodiments, the chemotherapy is administered in a cycling regimen of one or more 21-day cycles, wherein Day 1 is the first day of each cycle. In some embodiments, capecitabine is administered to the subject about twice daily (BID) on Days 1 to 14 of each 21-day cycle at a dose of about 500 to about 1000 mg/m² and oxaliplatin is administered to the subject on Day 1 of each 21-day cycle at a dose of about 100 to about 150 mg/m². In some embodiments, capecitabine is administered to the subject about twice daily (BID) on Days 1 to 14 of each 21-day cycle at a dose of about 500 mg/m², about 750 mg/m², about 850 mg/m², or about 1000 mg/m² and oxaliplatin is administered to the subject on Day 1 of each 21-day cycle at a dose of about 130 mg/m². In some embodiments, capecitabine is administered to the subject about twice daily (BID) on Days 1 to 14 of each 21-day cycle at a dose of about 850 to about 1000 mg/m² and oxaliplatin is administered to the subject on Day 1 of each 21-day cycle at a dose of about 130 mg/m². In some embodiments, capecitabine is administered to the subject about twice daily (BID) on Days 1 to 14 of each 21-day cycle at a dose of about 1000 mg/m² and oxaliplatin is administered to the subject on Day 1 of each 21-day cycle at a dose of about 130 mg/m².

In some embodiments, the chemotherapy is FOLFOX. In some embodiments, the chemotherapy comprises oxaliplatin, folinic acid or a therapeutic equivalent thereof, and fluorouracil. In some embodiments, the chemotherapy is administered in a cycling regimen, such as any described herein. In some embodiments, the chemotherapy is administered in a cycling regimen of one or more 28-day cycles, wherein Day 1 is the first day of each cycle. In some embodiments, oxaliplatin is administered to the subject on Days 1 and 15 of each 28-day cycle at a dose of about 50 mg/m² to about 90 mg/m²; folinic acid or a therapeutic equivalent thereof is administered to the subject on Days 1 and 15 of each 28-day cycle at a dose of about 200 mg/m² to about 450 mg/m²; and fluorouracil is administered to the subject as a bolus on Days 1 and 15 of each 28-day cycle at a dose of about 200 mg/m² to about 450 mg/m² and as a continuous infusion starting on Days 1 and 15 of each 28-day cycle at a dose of about of about 1600 mg/m²/48 hours to about 2500 mg/m²/48 hours. In some embodiments, oxaliplatin is administered to the subject on Days 1 and 15 of each 28-day cycle at a dose of about 50 mg/m², about 70 mg/m², or about 85 mg/m²; folinic acid or a therapeutic equivalent thereof is administered to the subject on Days 1 and 15 of each 28-day cycle at a dose of about 200 mg/m², about 300 mg/m², or about 400 mg/m²; and fluorouracil is administered to the subject as a bolus on Days 1 and 15 of each 28-day cycle at a dose of about 200 mg/m², about 300 mg/m², or about 400 mg/m² and as a continuous infusion starting on Days 1 and 15 of each 28-day cycle at a dose of about 1600 mg/m²/48 hours, about 2000 mg/m²/48 hours or about 2400 mg/m²/48 hours. In some embodiments, oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours.

In some instances, the multispecific antibody is administered in combination with nivolumab according to a dose and dosing schedule described herein. In some embodiments, the multispecific antibody is administered about once every two weeks at a dose of 30 mg, 90 mg, 250 mg, 500 mg, or 1000 mg in combination with nivolumab, wherein nivolumab is administered to the subject about once every two weeks at a dose of 240 mg, about once every three weeks at a dose of 360 mg, or about once every four weeks at a dose of 480 mg.

In some embodiments, the multispecific antibody is administered about once every two weeks at a dose of 30 mg, 90 mg, 250 mg, 500 mg, or 1000 mg in combination with nivolumab, wherein nivolumab is administered to the subject once every two to four weeks at a dose of or about 600 mg to or to about 1200 mg, inclusive. In some embodiments, the nivolumab is administered according to a dose and dosing schedule described herein. In some particular embodiments, the nivolumab is administered subcutaneously, and the nivolumab administered subcutaneously is co-formulated with hyaluronidase, such as any dose of hyaluronidase described herein.

In some embodiments, the multispecific antibody is administered about once every two weeks at a dose of 30 mg in combination with nivolumab, wherein nivolumab is administered to the subject about once every two weeks at a dose of 240 mg. In some embodiments, the multispecific antibody is administered about once every two weeks at a dose of 30 mg in combination with nivolumab, wherein nivolumab is administered to the subject about once every three weeks at a dose of 360 mg. In some embodiments, the multispecific antibody is administered about once every two weeks at a dose of 30 mg in combination with nivolumab, wherein nivolumab is administered to the subject about once every four weeks at a dose of 480 mg. In some embodiments, the multispecific antibody is administered about once every two weeks at a dose of 90 mg in combination with nivolumab, wherein nivolumab is administered to the subject about once every two weeks at a dose of 240 mg. In some embodiments, the multispecific antibody is administered about once every two weeks at a dose of 90 mg in combination with nivolumab, wherein nivolumab is administered to the subject about once every three weeks at a dose of 360 mg. In some embodiments, the multispecific antibody is administered about once every two weeks at a dose of 90 mg in combination with nivolumab, wherein nivolumab is administered to the subject about once every four weeks at a dose of 480 mg. In some embodiments, the multispecific antibody is administered about once every two weeks at a dose of 250 mg in combination with nivolumab, wherein nivolumab is administered to the subject about once every two weeks at a dose of 240 mg. In some embodiments, the multispecific antibody is administered about once every two weeks at a dose of 250 mg in combination with nivolumab, wherein nivolumab is administered to the subject about once every three weeks at a dose of 360 mg. In some embodiments, the multispecific antibody is administered about once every two weeks at a dose of 250 mg in combination with nivolumab, wherein nivolumab is administered to the subject about once every four weeks at a dose of 480 mg. In some embodiments, the multispecific antibody is administered about once every two weeks at a dose of 500 mg in combination with nivolumab, wherein nivolumab is administered to the subject about once every two weeks at a dose of 240 mg. In some embodiments, the multispecific antibody is administered about once every two weeks at a dose of 500 mg in combination with nivolumab, wherein nivolumab is administered to the subject about once every three weeks at a dose of 360 mg. In some embodiments, the multispecific antibody is administered about once every two weeks at a dose of 500 mg in combination with nivolumab, wherein nivolumab is administered to the subject about once every four weeks at a dose of 480 mg. In some embodiments, the multispecific antibody is administered about once every two weeks at a dose of 1000 mg in combination with nivolumab, wherein nivolumab is administered to the subject about once every two weeks at a dose of 240 mg. In some embodiments, the multispecific antibody is administered about once every two weeks at a dose of 1000 mg in combination with nivolumab, wherein nivolumab is administered to the subject about once every three weeks at a dose of 360 mg. In some embodiments, the multispecific antibody is administered about once every two weeks at a dose of 1000 mg in combination with nivolumab, wherein nivolumab is administered to the subject about once every four weeks at a dose of 480 mg.

In some embodiments, the multispecific antibody is administered in combination with nivolumab and a chemotherapy comprising capecitabine and oxliplatin in a cycling regimen of one or more 21-day cycles, wherein Day 1 is the first day of each cycle, wherein the multispecific antibody is administered to the subject on Day 1 of each 21-day cycle at a dose of about 10 mg to about 1000 mg; wherein nivolumab is administered to the subject on Day 1 of each 21-day cycle at a dose of about 360 mg; wherein capecitabine is administered to the subject about twice daily (BID) on Days 1 to 14 of each 21-day cycle at a dose of about 1000 mg/m²; and oxaliplatin is administered to the subject on Day 1 of each 21-day cycle at a dose of about 130 mg/m². In some embodiments, the multispecific antibody is administered to the subject at a dose of about 10 mg. In some embodiments, the multispecific antibody is administered to the subject at a dose of about 30 mg. In some embodiments, the multispecific antibody is administered to the subject at a dose of about 90 mg. In some embodiments, the multispecific antibody is administered to the subject at a dose of about 250 mg. In some embodiments, the multispecific antibody is administered to the subject at a dose of about 500 mg. In some embodiments, the multispecific antibody is administered to the subject at a dose of about 1000 mg.

In some embodiments, the multispecific antibody is administered in combination with nivolumab and a chemotherapy comprising oxaliplatin, folinic acid or a therapeutic equivalent thereof, and fluorouracil in a cycling regimen of one or more 28-day cycles, wherein Day 1 is the first day of each cycle, wherein the multispecific antibody is administered to the subject on Days 1 and 15 of each 28-day cycle at a dose of about 10 mg to about 1000 mg; wherein nivolumab is administered to the subject on Days 1 and 15 of each 28-day cycle at a dose of about 240 mg; wherein oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours. In some embodiments, the multispecific antibody is administered to the subject at a dose of about 10 mg. In some embodiments, the multispecific antibody is administered to the subject at a dose of about 30 mg. In some embodiments, the multispecific antibody is administered to the subject at a dose of about 90 mg. In some embodiments, the multispecific antibody is administered to the subject at a dose of about 250 mg. In some embodiments, the multispecific antibody is administered to the subject at a dose of about 500 mg. In some embodiments, the multispecific antibody is administered to the subject at a dose of about 1000 mg.

In some embodiments, the cancer is NSCLC and the subject has not yet received treatment. In some embodiments, the subject has a previously untreated metastatic cancer. In some embodiments, the cancer is metastatic NSCLC and the subject has not yet received treatment.

In some embodiments, the cancer is NSCLC and the subject previously received platinum doublet-based chemotherapy and then progressed or was intolerant to platinum doublet-based chemotherapy. In some embodiments, the subject previously received at least two prior lines of systemic therapy for advanced or metastatic disease and then progressed or was intolerant to at least two prior lines of systemic therapy for advanced or metastatic disease. In some embodiments, the subject has recurrent or progressive disease after completing platinum-based chemotherapy for local disease. In some embodiments, the subject has received previous treatment with a PD-1 therapy. In some embodiments, the subject has one or more mutations in a protein selected from EGFR, ALK, ROS1, and RET, and has received and progressed on, has been intolerant to, or was not a candidate for therapy with a tyrosine kinase inhibitor.

In some embodiments, the cancer is SCCHN and the SCCHN is of the oral cavity, pharynx, or larynx. In some embodiments, the cancer is SCCHN of the oral cavity, pharynx, or larynx and the subject has not yet received treatment. In some embodiments, the subject has a previously untreated metastatic cancer. In some embodiments, the cancer is metastatic SCCHN and the subject has not yet received treatment.

In some embodiments, the cancer is SCCHN of the oral cavity, pharynx, or larynx and the subject previously received a platinum-containing regimen and then progressed, or was intolerant to a platinum-containing regimen. In some embodiments, the subject has received previous treatment with a PD-1 therapy.

In some embodiments, the cancer is PDAC and the subject has not yet received treatment. In some embodiments, the subject has a previously untreated metastatic cancer. In some embodiments, the cancer is metastatic PDAC and the subject has not yet received treatment.

In some embodiments, the cancer is PDAC and the subject previously received at least one prior chemotherapy and then progressed; or was intolerant to at least one prior chemotherapy.

In some embodiments, the cancer is G/GEJC and the subject has not yet received treatment. In some embodiments, the subject has a previously untreated metastatic cancer. In some embodiments, the cancer is metastatic G/GEJC and the subject has not yet received treatment.

In some embodiments, the cancer is G/GEJC and the subject previously received at least one prior standard treatment regimen in the advanced or metastatic setting and then progressed, was intolerant to at least one prior standard treatment regimen in the advanced or metastatic setting, or has progressed within 6 months of adjuvant therapy. In some embodiments, the subject has received previous treatment with a PD-1 therapy. In some embodiments, the G/GEJC is human epidermal growth factor receptor 2 (HER2)-positive G/GEJC, and the subject has received prior treatment with a HER2 inhibitor. In some embodiments, the HER2 inhibitor is trastuzumab.

In some embodiments, the cancer is MSS CRC and the subject has not yet received treatment. In some embodiments, the subject has a previously untreated metastatic cancer. In some embodiments, the cancer is metastatic MSS CRC and the subject has not yet received treatment.

In some embodiments, the cancer is MSS CRC and the subject previously received at least one standard systemic therapy for metastatic and/or unresectable disease and then progressed, was intolerant to one standard systemic therapy for metastatic and/or unresectable disease, or has progressed within 6 months of adjuvant therapy. In some embodiments, the subject has received prior treatment with fluoropyrimidine, oxaliplatin, and irinotecan given as a single regimen or over multiple regimens. In some embodiments, the subject has proficient mismatch repair (MMR). In some embodiments, the subject has wild-type RAS and was previously treated with an anti-EGFR therapy. In some embodiments, the anti-EGFR therapy is cetuximab or panitumumab.

In some embodiments, treatment of the subject in need thereof, as described herein, is continued for about 1 month to about 24 months. In some embodiments, treatment of the subject in need thereof is continued for at least 1, 2, 3, 4, 5, 6, 7, 8, 9, 10, 11, 12, 16, 20, or 24 months. In some embodiments, treatment of the subject in need thereof is continued for 1, 2, 3, 4, 5, 6, 7, 8, 9, 10, 11, 12, 13, 14, 15, 16, 17, 18, 19, 20, 21, 22, 23, or 24 months. In some embodiments, treatment of the subject in need thereof is continued for at least 1, 2, 3, 4, 5, or 6 months. In some embodiments, treatment of the subject in need thereof is continued until the subject achieves a complete response.

In some embodiments, the subject is administered the multispecific antibody for about 1 month to about 24 months. In some embodiments, the subject is administered the multispecific antibody for at least 1, 2, 3, 4, 5, 6, 7, 8, 9, 10, 11, 12, 16, 20, or 24 months. In some embodiments, the subject is administered the multispecific antibody for 1, 2, 3, 4, 5, 6, 7, 8, 9, 10, 11, 12, 13, 14, 15, 16, 17, 18, 19, 20, 21, 22, 23, or 24 months. In some embodiments, the subject is administered the multispecific antibody for at least 1, 2, 3, 4, 5, or 6 months. In some embodiments, the multispecific antibody is administered until the subject achieves a complete response. In some embodiments, the multispecific antibody is administered in combination with a PD-1 therapy for some portion of or the entire treatment period. In some embodiments, the multispecific antibody is administered in combination with a PD-1 therapy for the entire treatment period. In some embodiments, the PD-1 therapy is nivolumab.

In some embodiments, the multispecific antibody is administered to the subject by intravenous administration. In some embodiments, the intravenous administration is completed over about 10, 20, 30, 40, 50, or 60 minutes. In some embodiments, the intravenous administration is completed over 10, 20, 30, 40, 50, or 60 minutes. In some embodiments, the intravenous administration is completed over about 30 minutes. In some embodiments, the intravenous administration is completed over 30 minutes. In some embodiments, the multispecific antibody is administered to the subject by subcutaneous administration.

In some embodiments, the subject is administered the multispecific antibody for about 1 month to about 24 months in combination with a PD-1 therapy. In some embodiments, the subject is administered the multispecific antibody for at least 1, 2, 3, 4, 5, 6, 7, 8, 9, 10, 11, 12, 16, 20, or 24 months in combination with a PD-1 therapy. In some embodiments, the subject is administered the multispecific antibody for 1, 2, 3, 4, 5, 6, 7, 8, 9, 10, 11, 12, 13, 14, 15, 16, 17, 18, 19, 20, 21, 22, 23, or 24 months in combination with a PD-1 therapy. In some embodiments, the subject is administered the multispecific antibody for at least 1, 2, 3, 4, 5, or 6 months in combination with a PD-1 therapy. In some embodiments, the PD-1 therapy is nivolumab. In some embodiments, the multispecific antibody and nivolumab are administered until the subject achieves a complete response.

In some embodiments, the PD-1 therapy is administered to the subject before the multispecific antibody is administered to the subject. In some embodiments, the multispecific antibody is administered to the subject before the PD-1 therapy is administered to the subject.

Also provided herein is a multispecific antibody for use in any one of the methods provided herein, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16. The antibody for use can be administered to subjects as described herein and in dosage schedules as described herein.

Also provided herein is use of a multispecific antibody in the manufacture of a medicament for any one of the methods provided herein, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16. The use can comprise administration to subjects as described herein and in dosage schedules as described herein.

### B. Routes of Administration and Carriers

Provided herein are compositions (e.g., pharmaceutical compositions) comprising an antibody provided herein and one or more pharmaceutically acceptable carriers.

In various embodiments, an antibody provided herein may be administered in vivo by various routes, including, but not limited to, oral, intra-arterial, parenteral (including intravenous and subcutaneous), intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations, such as liquid formulations or formulations suitable for injections, inhalations, and the like. Alternatively, in some embodiments, the composition may be provided as a lyophilized powder that may be reconstituted upon addition of an appropriate liquid or carrier, for example, sterile water. The appropriate formulation and route of administration may be selected according to the intended application.

### C. Disease Indications

In some embodiments, the methods provided herein comprises treating cancer in a subject, where the cancer expresses fibroblast activation protein alpha (FAP). FAP, which is expressed at low levels in healthy adult tissue, is upregulated in the tumor stroma. The tumor stroma, which comprises connective tissue, blood vessels, and inflammatory cells, serves as the microenvironment interface between malignant and healthy cells. In some embodiments, the cancer treated by the provided methods is an FAP-expressing cancer. In some embodiments, the cancer is a solid tumor. Solid tumors expressing higher levels of FAP include, but are not limited to, lung, colorectal, pancreatic, gastric, and others. In some embodiments, the cancer is selected from non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN). In some embodiments, the cancer is NSCLC. In some embodiments, the cancer is MSS CRC. In some embodiments, the cancer is PDAC. In some embodiments, the cancer is G/GEJC. In some embodiments, the cancer is SCCHN.

### D. Patient Populations

In some embodiments, the multispecific antibody provided herein is used to treat a patient or subject having cancer. In some embodiments, the patient having cancer is treated with the multispecific antibody in combination with a PD-1 therapy (e.g., nivolumab or other antibody binding to PD-1 or PD-L1), as described, for example, in Section VIII.A. In some embodiments, the patient has cancer with elevated fibroblast activation protein alpha (FAP) expression, as described, for example, in Section VIII.C. In some embodiments, the patient has a solid tumor.

In some embodiments, the patient has cancer where the cancer is selected from non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN). In some embodiments, the patient has NSCLC. In some embodiments, the patient has MSS CRC. In some embodiments, the patient has PDAC. In some embodiments, the patient has G/GEJC. In some embodiments, the patient has human epidermal growth factor receptor 2 (HER2)-positive G/GEJC. In some embodiments, the patient has SCCHN. In some embodiments, the patient has SCCHN of the oral cavity, pharynx, or larynx.

In some embodiments, the patient has not yet received treatment. In some embodiments, the patient is a previously untreated patient with metastatic cancer. In some embodiments, the patient has NSCLC and the patient has not yet received treatment. In some embodiments, the patient has metastatic NSCLC and the patient has not yet received treatment. In some embodiments, the patient has MSS CRC and the patient has not yet received treatment. In some embodiments, the patient has metastatic MSS CRC and the patient has not yet received treatment. In some embodiments, the patient has PDAC and the patient has not yet received treatment. In some embodiments, the patient has metastatic PDAC and the patient has not yet received treatment. In some embodiments, the patient has G/GEJC and the patient has not yet received treatment. In some embodiments, the patient has metastatic G/GEJC and the patient has not yet received treatment. In some embodiments, the patient has SCCHN and the patient has not yet received treatment. In some embodiments, the patient has metastatic SCCHN and the patient has not yet received treatment.

In some embodiments, the patient has received previous treatment. In some embodiments, the patient received previous treatment with a PD-1 therapy. In some embodiments, the patient received previous treatment and progressed. In some embodiments, the patient progressed within about 3, 4, 5 or 6 months of adjuvant therapy. In some embodiments, the patient progressed within 6 months of adjuvant therapy. In some embodiments, the patient was intolerant to at least one prior standard treatment regimen in the advanced or metastatic setting. In some embodiments, the patient was intolerant to 1, 2, 3, 4, 5, 6, 7, or 8 prior standard treatment regimens in the advanced or metastatic setting.

In some embodiments, the patient has NSCLC and the patient previously received platinum doublet-based chemotherapy and then progressed or was intolerant to platinum doublet-based chemotherapy. In some embodiments, the patient previously received at least two prior lines of systemic therapy for advanced or metastatic disease and then progressed. In some embodiments, the patient previously received 2, 3, 4, 5, 6, 7, or 8 prior lines of systemic therapy for advanced or metastatic disease and then progressed. In some embodiments, the patient was intolerant to at least two prior lines of systemic therapy for advanced or metastatic disease. In some embodiments, the patient was intolerant to 2, 3, 4, 5, 6, 7, or 8 prior lines of systemic therapy for advanced or metastatic disease. In some embodiments, the patient has recurrent disease after completing platinum-based chemotherapy for local disease. In some embodiments, the patient has progressive disease after completing platinum-based chemotherapy for local disease. In some embodiments, the patient has received previous treatment with a PD-1 therapy. In some embodiments, the patient has one or more mutations in a tyrosine kinase. In some embodiments, the patient has one or more mutations in a protein selected from EGFR, ALK, ROS1, and RET. In some embodiments, the patient has two or more mutations in a protein selected from EGFR, ALK, ROS1, and RET. In some embodiments, the patient has three or more mutations in a protein selected from EGFR, ALK, ROS1, and RET. In some embodiments, the patient has mutations in EGFR, ALK, ROS1, and RET. In some embodiments, the patient has received and progressed on a tyrosine kinase inhibitor. In some embodiments, the patient has been intolerant to a tyrosine kinase inhibitor. In some embodiments, the patient was not a candidate for therapy with a tyrosine kinase inhibitor.

In some embodiments, the patient has SCCHN, where the SCCHN is of the oral cavity, pharynx, or larynx. In some embodiments, the patient has SCCHN of the oral cavity. In some embodiments, the patient has SCCHN of the pharynx. In some embodiments, the patient has SCCHN of the larynx. In some embodiments, the patient previously received a platinum-containing regimen and then progressed. In some embodiments, the patient was intolerant to a platinum-containing regimen. In some embodiments, the patient has received previous treatment with a PD-1 therapy. In some embodiments, the patient is negative for oropharyngeal cancers related to human papillomavirus (HPV). In some embodiments, the patient is positive for oropharyngeal cancers related to HPV. In some embodiments, the HPV status is determined using p16 IHC or HPV PCR.

In some embodiments, the patient has PDAC and the patient previously received at least one prior chemotherapy and then progressed. In some embodiments, the patient previously received 1, 2, 3, 4, 5, 6, 7, or 8 prior chemotherapies and then progressed. In some embodiments, the patient was intolerant to at least one prior chemotherapy. In some embodiments, the patient was intolerant to 1, 2, 3, 4, 5, 6, 7, or 8 prior chemotherapies.

In some embodiments, the patient has G/GEJC and the patient previously received at least one prior standard treatment regimen in the advanced or metastatic setting and then progressed. In some embodiments, the patient previously received 1, 2, 3, 4, 5, 6, 7, or 8 prior standard treatment regimens in the advanced or metastatic setting and then progressed. In some embodiments, the patient was intolerant to at least one prior standard treatment regimen in the advanced or metastatic setting. In some embodiments, the patient was intolerant to 1, 2, 3, 4, 5, 6, 7, or 8 prior standard treatment regimens in the advanced or metastatic setting. In some embodiments, the patient has progressed within about 3, 4, 5, or 6 months of adjuvant therapy. In some embodiments, the patient has progressed within 6 months of adjuvant therapy. In some embodiments, the patient has received previous treatment with a PD-1 therapy. In some embodiments, the patient has human epidermal growth factor receptor 2 (HER2)-positive G/GEJC. In some embodiments, the patient has received prior treatment with a HER2 inhibitor. In some embodiments, the HER2 inhibitor is trastuzumab. In some embodiments, the patient has proficient mismatch repair (MMR). In some embodiments, the patient microsatellite stable (MSS).

In some embodiments, the patient has MSS CRC and the patient previously received at least one standard systemic therapy for metastatic and/or unresectable disease and then progressed. In some embodiments, the patient previously received 1, 2, 3, 4, 5, 6, 7, or 8 standard systemic therapies for metastatic and/or unresectable disease and then progressed. In some embodiments, the patient was intolerant to one standard systemic therapy for metastatic and/or unresectable disease. In some embodiments, the patient has progressed within about 3, 4, 5, or 6 months of adjuvant therapy. In some embodiments, the patient has progressed within 6 months of adjuvant therapy. In some embodiments, the patient has received prior treatment with fluoropyrimidine, oxaliplatin, and irinotecan given as a single regimen. In some embodiments, the patient has received prior treatment with fluoropyrimidine, oxaliplatin, and irinotecan given over multiple regimens. In some embodiments, the patient is microsatellite stable (MSS). In some embodiments, the patient is mismatch repair (MMR) proficient. In some embodiments, the patient is MSS and MMR proficient. In some embodiments, the patient is not microsatellite instability (MSI)-high, MSI-low, or MMR deficient. In some embodiments, the patient has a known status of KRAS, NRAS, and BRAF. In some embodiments, the patient has wild-type RAS and was previously treated with an anti-EGFR therapy. In some embodiments, the anti-EGFR therapy is cetuximab. In some embodiments, the anti-EGFR therapy is panitumumab.

In some embodiments, the patient has histologically or cytologically confirmed locally advanced unresectable, metastatic, or recurrent malignant tumor selected from NSCLC, MSS CRC, PDAC, G/GEJC, and SCCHN. In some embodiments, the patient has measurable disease by Response Evaluation Criteria in Solid Tumors version 1.1 (RECIST v1.1). In some embodiments, the patient has an Eastern Cooperative Oncology Group Performance Status of 0 or 1. In some embodiments, the patient is at least 18 years of age or of legal age of consent in the jurisdiction in which the study is taking place at the time of signing the ICF.

### IX. EXAMPLES

### Example 1: Generation of Anti-CD40 Antibodies

Screening and discovery of fully human immunoglobulin G (IgG) antibodies against human CD40 was performed by immunization of BMS HCo38/42 humanized mice with CHO cells expressing human CD40. Sixty mouse hybridomas were screened by single point surface plasmon resonance (SPR) for binding to the extracellular domains of recombinant human and cyno CD40. Twelve human and cyno cross-reactive CD40 positive mouse hybridoma clones were sequenced, and recombinant fully human IgG1 antibodies were produced. Purified CD40 antibodies were screened for B cell agonism and CD40 ligand blocking by SPR. Nine CD40 antibodies were found to be CD40 agonists, and were also CD40-ligand (CD40L) non-blocking, with a range of affinities (2-150 nM). These nine CD40 agonists were reformatted as monovalent 1+1 CD40×FAP bispecific antibodies and were screened to identify FAP driven clustering dependent CD40 agonism. The clone 9H3 was identified as a potent FAP dependent CD40 agonist and was further optimized to remove sequence liabilities.

### Example 2: Generation of Anti-FAP Antibodies

Screening and discovery of fully human immunoglobulin G (IgG) antibodies against human FAP was performed by phage selection using the SuperHuman 2.0 synthetic naive scFv library and panning against recombinant human and mouse extracellular domains and HEK-293 human FAP overexpression cell lines. Positive clones were sequenced and scFvs were produced from bacterial periplasmic extracts. Purified scFvs were screened for cross-reactive binding to human, cyno, and mouse FAP by ELISA, while displaying no binding to the related proline selective peptidase dipeptidyl peptidase 4 (DPP4). Eighty-two human, cyno, and mouse cross-reactive FAP positive clones were reformatted and produced as recombinant fully human IgG1 antibodies. Purified FAP antibodies were used to determine relative affinities using single point Mirror Ball measurements, followed by dose titrations for affinity measurement by FACS. Three clones demonstrated significant human, cyno, and mouse cross-reactivity and were selected for affinity maturation by phage display using a CDR focused library. SPR affinity measurements identified three clones with 10-fold greater affinities relative to each of the three parental antibodies for human and cyno FAP. These clones were selected for reformatting as CD40×FAP bispecific antibodies and were screened to identify the most potent FAP driven clustering dependent CD40 agonism. The anti-FAP clone LP62 was found to drive the greatest CD40 agonism, while possessing ideal biophysical traits.

### Example 3: Generation of Trivalent Bispecific Anti-CD40/FAP Antibody

Various formats of bispecific anti-CD40/FAP antibodies were assessed, including, for example, formats comprising one CD40 binding domain and one FAP binding domain, or two CD40 binding domains and one FAP binding domain. After characterization of various anti-CD40 and anti-FAP antibody clones in various formats, CD40.9H3×FAP.LP62 bispecific antibody (CD40.9H3×FAP.LP62) was selected for further development.

CD40.9H3×FAP.LP62 is a humanized immunoglobulin G1 (IgG1) bispecific antibody comprised of a bivalent arm that recognizes CD40 and a single chain variable fragment (scFv) that recognizes fibroblast activation protein (FAP). The IgG1 fragment crystallizable (Fc) region of CD40.9H3×FAP.LP62 contains three amino acid substitutions (L234A, L235A, D265S) that attenuate binding to Fc gamma receptors (FcγR). The Fc of each heavy chain also includes mutations to facilitate heterodimerization (T350V, T366L, K392L, and T394W in the first heavy chain, and T350V, L351Y, F405A, and Y407V in the second heavy chain). CD40.9H3×FAP.LP62 therefore comprises three different polypeptide sequences in a ratio of 1:1:2—a first heavy chain (SEQ ID NO: 15), a second heavy chain fused to a scFv (SEQ ID NO: 34) and two light chains (SEQ ID NO: 16).

### Example 4: Binding Characterization of CD40.9H3×FAP.LP62

CD40.9H3×FAP.LP62 and an analog of RO7300490 (see SEQ ID NOs: 39-42) binding to human and cynomolgus (cyno) CD40 and FAP was evaluated by surface plasmon resonance (SPR) using human and cyno CD40 and FAP proteins. CD40.9H3×FAP.LP62 bound to human CD40 and FAP in a dose-dependent manner with a mean (±SD) equilibrium dissociation constant (KD) of 9.2±0.6×10⁻⁸ M and 1.96±0.07×10⁻⁹ M, respectively. The rates of association and dissociation of CD40.9H3×FAP.LP62 and the analog of RO7300490 to human CD40 and FAP are summarized in Table 2. The rates of association and dissociation of CD40.9H3×FAP.LP62 to cyno CD40 and FAP are also summarized in Table 2.

SPR studies also demonstrated that CD40.9H3×FAP.LP62 does not block binding of CD40 to its physiologic ligand, CD40L (data not shown).

TABLE 2
Binding Kinetics of CD40.9H3 × FAP.LP62 and
the analog of RO7300490 to Human and Cyno CD40 and FAP

| Species/Construct | Target | ka (1/Ms) | kd (1/s) | KD (M) |
|---|---|---|---|---|
| Human/ | CD40 | 5.7 ± 0.5 × 10⁴ | 5.2 ± 0.1 × 10⁻³ | 9.2 ± 0.6 × 10⁻⁸ |
| CD40.9H3 × FAP.LP62 | | | | |
| Human/ | FAP | 7.5 ± 0.2 × 10⁵ | 1.47 ± 0.01 × 10⁻³ | 1.96 ± 0.07 × 10⁻⁹ |
| CD40.9H3 × FAP.LP62 | | | | |
| Human/analog of | CD40 | 1.13 × 10⁶ | 5.55 × 10⁻³ | 4.9 × 10⁻⁹ |
| RO7300490 | | | | |
| Human/analog of | FAP | 1.82 × 10⁵ | 4.83 × 10⁻⁵ | 2.65 × 10⁻¹⁰ |
| RO7300490 | | | | |
| Cyno/ | CD40 | 5.3 ± 0.4 × 10⁴ | 5.2 ± 0.1 × 10⁻³ | 9.7 ± 0.6 × 10⁻⁸ |
| CD40.9H3 × FAP.LP62 | | | | |
| Cyno/ | FAP | 5.0 ± 0.3 × 10⁵ | 2.76 ± 0.02 × 10⁻³ | 5.6 ± 0.3 × 10⁻⁹ |
| CD40.9H3 × FAP.LP62 | | | | |

Binding of CD40.9H3×FAP.LP62 was also evaluated across several human and cyno CD40- and FAP expressing cells compared to isotype controls. Dose-dependent binding was observed on human macrophages, human B cells, and human FAP-expressing human embryonic kidney (HEK) cells. Mean (+SD) half maximal effective concentrations (EC50) for binding to cells are summarized in Table 3.

TABLE 3
CD40.9H3 × FAP.LP62 Binds to Human
and Cyno CD40- and FAP-Expressing Cells

| Species | Cell type | EC50 (nM) |
|---|---|---|
| Human | CD40-positive macrophage | 11.91 ± 1.52 |
| Human | CD40-positive B cell | 7.53 ± 1.87 |
| Human | FAP-positive HEK cells | 3.72 ± 1.78 |
| Cyno | CD40-positive B cell | 4.69 ± 1.65 |
| Cyno | FAP-positive HEK cells | 3.60 ± 2.23 |

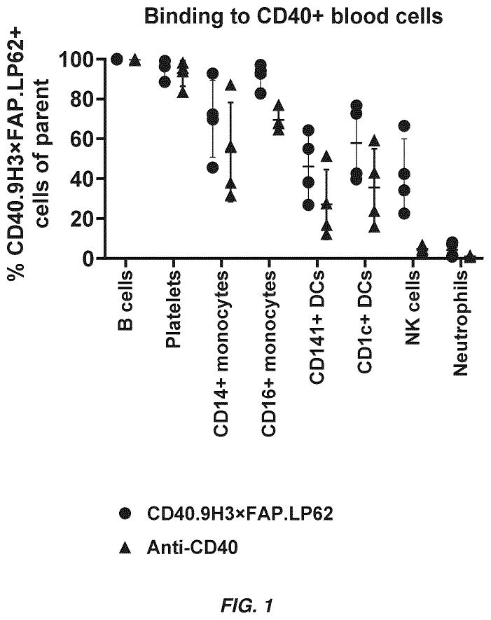

CD40.9H3×FAP.LP62 was evaluated for binding to cells found in human blood. Commercially available anti-CD40 and anti-FAP antibodies were used as comparators. Healthy human blood was analyzed for CD40.9H3×FAP.LP62, anti-CD40 comparator antibody, and anti-FAP comparator antibody binding (n=4 donors across 2 independent experiments). The results are shown in FIG. 1. Each data point represents a single donor for each cell population. Positively stained cells were reflected as a percent of parent using the following gating strategy: B cell (CD19+CD20+), platelets (CD41a+), CD14+ monocytes (CD14+CD16−), CD16+ monocytes (CD14− CD16+), CD141+ DCs (CD141+), CD1c+ DCs (CD1c+), NK cells (CD56+), and neutrophils (CD15+CD16+). CD40 expression was detected on B cells, platelets, CD14+ monocytes, CD16+ monocytes, CD141+ DCs, CD1c+ DCs, NK cells, and neutrophils. No FAP expression was detected on any human blood cells (data not shown). CD40.9H3×FAP.LP62 bound to all of these cell types to a similar or greater extent than a commercially-available anti-CD40 antibody, demonstrating that CD40.9H3×FAP.LP62 binds to CD40 specifically in human blood. See FIG. 1.

### Example 5: Attenuated Effector Function of CD40.9H3×FAP.LP62

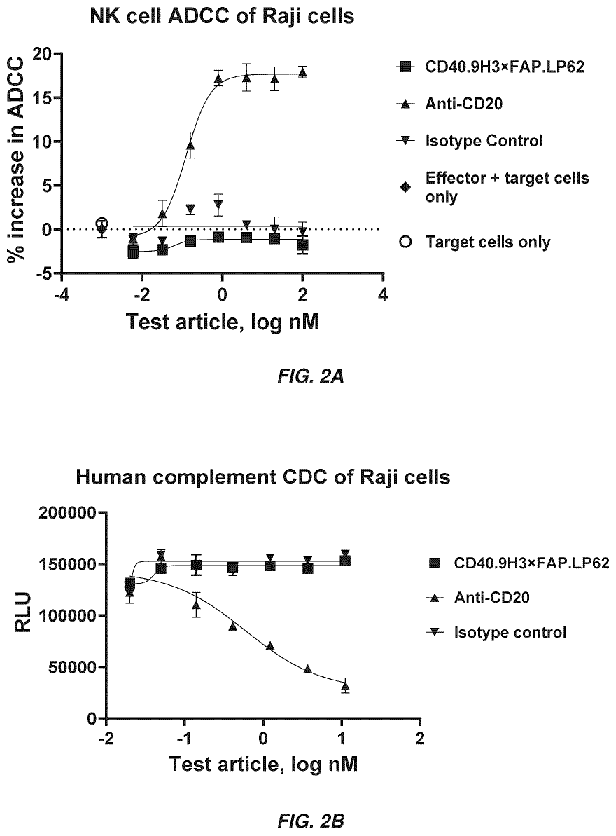

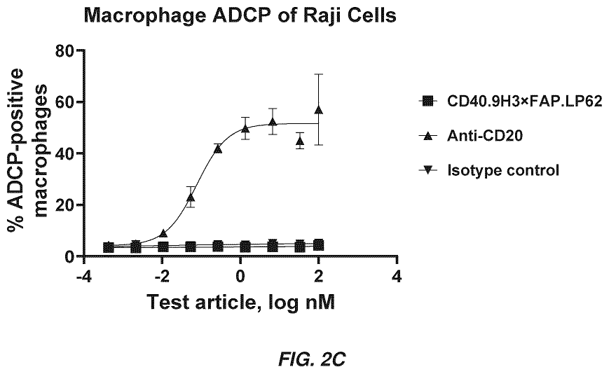

CD40.9H3×FAP.LP62 was designed to restrict its activity to FAP-expressing cells while minimizing Fc gamma receptor-mediated activity through utilization of the L234A, L235A, D265S (AAS) substitutions of the human IgG1 Fc. The analog of RO7300490 utilizes the L234A, L235A, P329G (AAG) substitutions of the human IgG1 Fc to achieve effector activity attenuation. CD40-positive Raji cells were used as targets to evaluate the Fc function of CD40.9H3×FAP.LP62 in ADCC killing, CDC killing, and ADCP by co-culture with human NK cells, complement, and macrophages from healthy human donors, respectively. Anti-CD20 (Raji target cells are CD20-positive) and isotype control antibodies were included as controls. Whereas no NK cell mediated ADCC or CDC activities were detected with CD40.9H3×FAP.LP62 and the anti-HEL isotype control antibody, Raji cells were efficiently killed with the anti-CD20 positive control antibody (n=3 donors across 3 independent experiments and n=3 independent experiments, respectively). See FIG. 2A-2C. Similarly, the anti-CD20 positive control antibody targeted Raji cells for phagocytosis by macrophages while no such activity was observed with CD40.9H3×FAP.LP62 or the anti-HEL isotype control (n=2 donors across 2 independent experiments, respectively).

Binding of CD40.9H3×FAP.LP62 and the analog of RO7300490 to various Fc gamma receptors (FcγR1 (CD64), FcγRIIIA (CD16a), FcγRIIIB (CD16b), FcγRIIA (CD32a), FcγRIIB (CD32b)) was determined in an analyte/capture assay in vitro. Briefly, an SPR assay was performed using a Biacore S200 with a Biacore Series S Protein A chip (Cytiva). All antibodies were prepared at 4.0 μg/ml in 1×HBS-EP+ running buffer (Cytiva) and captured onto flow cells 2, 3, and 4 through a 60 second contact time at 10 μl/min. An analyte series of the FcγRs (BMS In-House Produced) were prepared at 10,000 nM in 1×HBS-EP+ with eight, three-fold dilutions. Kinetic measurements were conducted with a 120 second association followed by a 120 second dissociation at 30 μl/min. Surface regeneration was preformed by 60 second contact time of 50 mM Glycine pH 1.5 solution (Cytiva) at 30 μl/min. Data was processed in Biacore Insight Evaluation Version 5 (Cytiva). Sensorgrams were double referenced and globally fit to a 1:1 Langmuir model were applicable. Where 1:1 model was not sufficient to describe the interaction (fast-on and fast-off rates), steady state equilibrium fitting was used to determine the equilibrium constant.

CD40.9H3×FAP.LP62 showed no binding to any of the Fc gamma receptors tested, while the analog of RO7300490 showed binding to FcγRI (CD64), with a K_{D} of 2.55×10⁻⁶ M. The Fc domain of CD40.9H3×FAP.LP62 is therefore fully attenuated, while the analog of RO7300490 retains binding to at least FcγR1.

### Example 6: CD40.9H3×FAP.LP62 Activates Human Dendritic Cells and Macrophages in a FAP-Dependent Manner

The activity of CD40.9H3×FAP.LP62 was evaluated for the ability to induce IL12p40 secretion in a human dendritic cell (DC) and HFF1 co-culture activity assay. DCs were co-cultured with either HFF1 or HFF1 FAP KO cells. HFF1 cells express FAP receptors on their cell surface with a mean (+SD) of 50617±19408 that is comparable to levels observed in cancer-associated fibroblasts from human tumors.

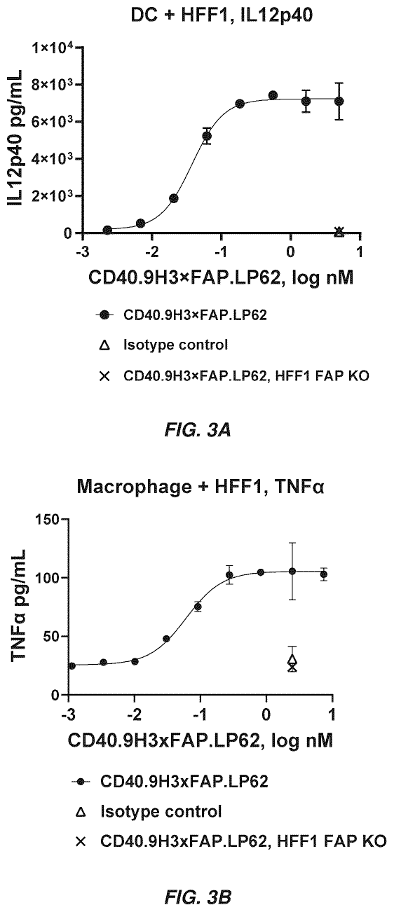

The results are shown in FIG. 3A. Dose-dependent IL12p40 secretion was observed with a mean (+SD) EC50 concentration of 0.079±0.043 nM (n=8 donors across 4 independent experiments). No IL12p40 secretion was observed when DCs were co-cultured with HFF1 FAP KO cells (absence of FAP) or anti-HEL×FAP isotype control antibody. Similar FAP dependent and dose-dependent responses were observed from CD40-positive macrophages co-cultured with HFF1 cells in the presence of CD40.9H3×FAP.LP62 with a mean (+SD) EC50 concentration for TNFα release of 0.12±0.05 nM (n=6 donors across 3 independent experiments). See FIG. 3B.

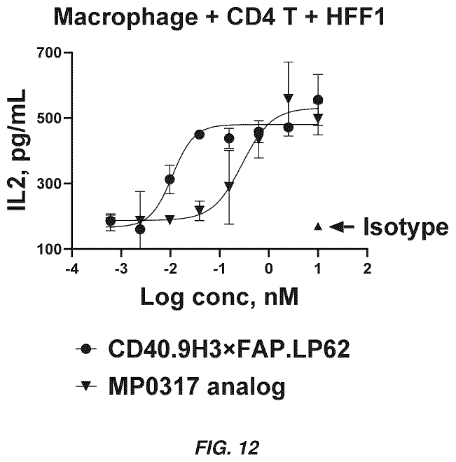

Macrophages can activate CD4+ T cells, so the ability of CD40.9H3×FAP.LP62-activated macrophages to induce CD4+ T cell function was also evaluated. In a tri-culture assay of macrophages, HFF1 cells, and CD4+ T cells, CD40.9H3×FAP.LP62 induced secretion of the T cell-specific cytokine IL2 in a dose-dependent manner, while no IL2 was observed with the anti-HEL×FAP isotype control (n=3 donors across 2 independent experiments, FIG. 12).

### Example 7: Mouse Surrogate mCD40×FAP.LP62 Characterization

CD40.9H3×FAP.LP62 does not bind to mouse CD40. Therefore, a 2+1 anti-mouse CD40×FAP bispecific antibody (mCD40×FAP.LP62) having a similar structure as CD40.9H3×FAP.LP62, with the same LP62 anti-FAP scFv, was developed as a surrogate for use in mouse tumor efficacy studies. mCD40×FAP.LP62 was comprehensively assessed through binding and in vitro functional assays and found to be comparable to CD40.9H3×FAP.LP62. mCD40×FAP.LP62 contains three amino acid substitutions (L234A, L235A, P329G) in the mouse IgG2a Fc backbone that attenuate binding to Fc gamma receptors (FcγR). Thus, similar to CD40.9H3×FAP.LP62, CD40 agonism is mediated by FAP-dependent clustering of multiple mCD40×FAP.LP62 antibodies.

mCD40×FAP.LP62 binding to mouse CD40 and FAP were evaluated by SPR using mouse CD40 and FAP proteins. mCD40×FAP.LP62 bound to mouse CD40 and FAP, similar to CD40.9H3×FAP.LP62, in a dose-dependent manner with a mean (+SD) equilibrium dissociation constant (K_{D}) of 4.58±0.09×10⁻⁸ M and 3.4±0.2×10⁻⁹ M, respectively. Binding of mCD40×FAP.LP62 was also evaluated on different mouse CD40- and FAP-expressing cells compared to isotype controls and commercially available comparator anti-CD40 and anti-FAP antibodies. Comparable to CD40.9H3×FAP.LP62, dose-dependent binding was observed on murine macrophages, murine B cells, and mouse FAP-expressing HEK cells (data not shown). Mean (+SD) half maximal effective concentrations (EC50) for binding to cells are summarized in Table 4.

TABLE 4
mCD40 × FAP.LP62 Binds to
Mouse CD40- and FAP-Expressing Cells

| Species | Cell type | EC50 (nM) |
|---|---|---|
| Mouse | CD40-positive macrophage | 7.30 ± 5.48 |
| Mouse | CD40-positive B cell | 12.11 ± 9.88 |
| Mouse | FAP-positive HEK cells | 3.17 ± 0.94 |

### Example 8: mCD40×FAP.LP62 Activates CD40 in a FAP-Dependent Manner

To confirm comparable functional activity of mCD40×FAP.LP62 to CD40.9H3×FAP.LP62, increasing concentrations of mCD40×FAP.LP62 were added to primary murine bone marrow-derived macrophages (BMDM) co-cultured with MC38 cells that express FAP (MC38-FAP) or MC38-FAP KO cells.

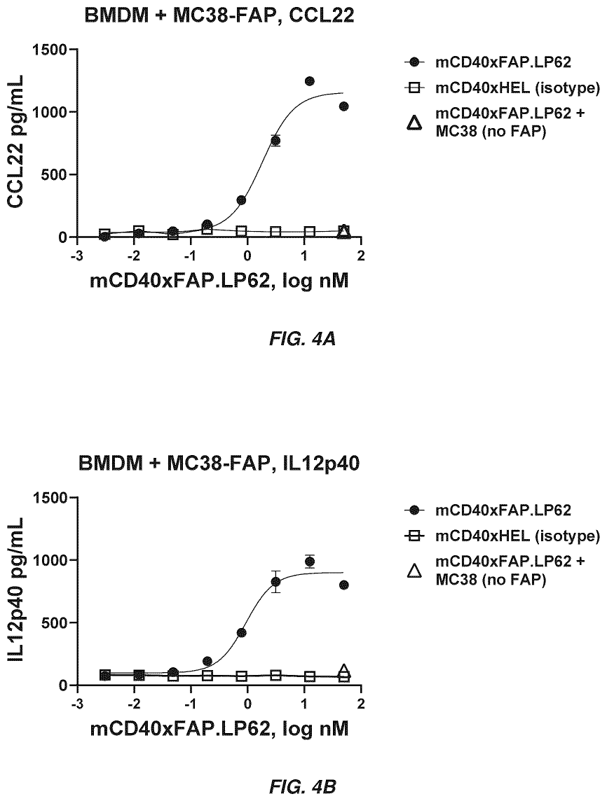

The FAP expressed on the surface of MC38-FAP cells [mean (±SD) of 27943±9551 FAP receptors (data not shown)] enables mCD40×FAP.LP62 clustering to drive CD40 activation and is comparable to levels found in patient tumors [mean (±SD) of 35024±25653 FAP receptors (data not shown)]. The CCL22 and IL12p40 cytokines downstream of CD40 activation were induced in a mCD40×FAP.LP62 dose-dependent manner with mean (±SD) EC50 concentrations of 1.02±0.13 nM and 1.43±0.56 nM, respectively (n=4 pooled mice across 2 independent experiments). See FIGS. 4A-4B. mCD40×FAP.LP62 activity was dependent on FAP as no CCL22 and IL12p40 were detected upon co-culture with MC38 cells that did not express FAP, nor with mCD40×HEL, an anti-mCD40/anti-hen egg lysozyme isotype-matched bispecific antibody. Collectively, these data support the use of mCD40×FAP.LP62 as a relevant in vivo surrogate for CD40.9H3×FAP.LP62 in mice.

### Example 9: Antitumor Efficacy and Pharmacodynamic Activity of mCD40×FAP.LP62 in KPCY Syngeneic Mouse Tumor Model

The antitumor effects of mCD40×FAP.LP62 were evaluated in the KPCY tumor-bearing mouse model, which harbors tumor stromal cells expressing FAP levels comparable to human tumors (data not shown). Tumor volumes following a single dose of mCD40×FAP.LP62 as monotherapy was monitored along with immune cell phenotyping and cytokine changes.

C57/BL6 mice were implanted with 5×10⁵ KPCY cells subcutaneously, randomized into treatment groups of 10 mice each when average tumor volumes reached 75 mm³ to 150 mm³, and dosed via the intraperitoneal (IP) route with either mCD40×FAP.LP62 or an anti-hen egg lysozyme isotype bispecific antibody control, mCD40×HEL. Because this isotype antibody contains the same anti-CD40 antibody arms as mCD40×FAP.LP62, this control enabled the evaluation of FAP binding-dependent CD40 agonist activity in vivo.

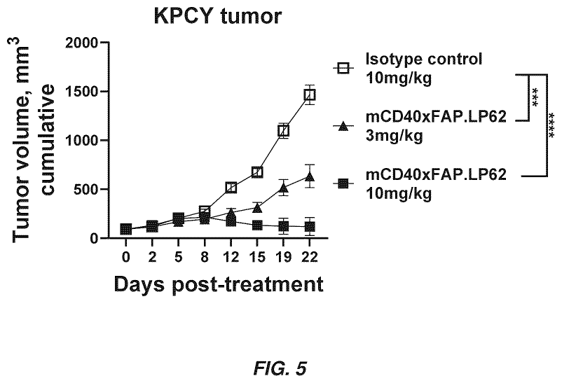

The results are shown in FIG. 5. Significant single agent activity of mCD40×FAP.LP62 was observed at the 10 mg/kg dose, with 5 of 10 tumor-free mice as well as at a lower dose of 3 mg/kg (n=10 mice per group across 2 independent experiments). Mean tumor volumes are summarized in Table 5.

TABLE 5
mCD40 × FAP.LP62 Drives Single
Agent Efficacy in KPCY Tumor Models

| | Mean Tumor Volume (mm³) ± SD | |
|---|---|---|
| | Day 0 Post | Day 22 Post |
| Antibody Treatment | Treatment | Treatment |
| Isotype control, 10 mg/kg | 93.5 ± 18.2 | 1465.8 ± 316.6 |
| mCD40 × FAP.LP62, 3 mg/kg | 92.9 ± 18.9 | 634.4 ± 369.1 |
| mCD40 × FAP.LP62, 10 mg/kg | 93.2 ± 18.9 | 119 ± 291.1 |

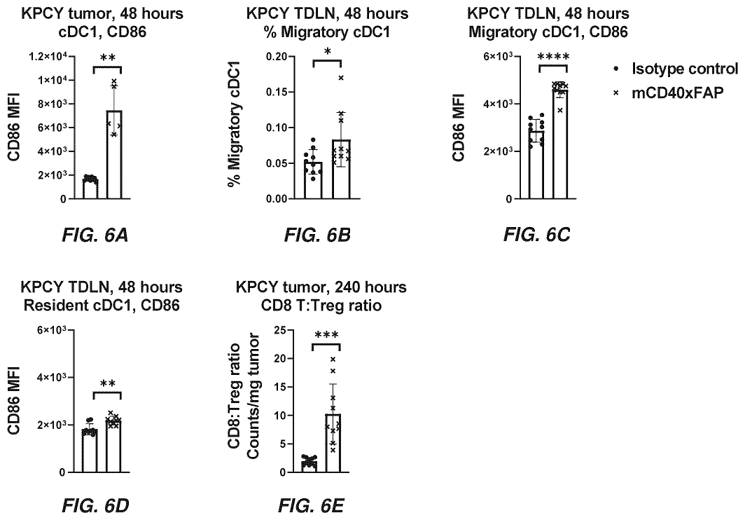

Immune profiling of KPCY tumor-bearing mice dosed with 10 mg/kg mCD40×FAP.LP62 was subsequently performed. KPCY mice were treated with mCD40×FAP.LP62 or anti-KLH isotype control (10 mg/kg, QDx1). Tumors and tumor-draining lymph nodes (TDLNs) were profiled by flow cytometry at 48 hours and 240 hours post-treatment (n=10 mice per group across 2 independent experiments). Cell populations for which there were <100 cells were excluded from analyses. This experiment demonstrated that tumoral cross-presenting dendritic cells (cDC1) upregulated expression of CD86, a costimulatory receptor and marker of cDC1 activation, as soon as 48 hours post-treatment compared to isotype control-treated mice (FIG. 6A). Additionally, the percent of activated cDC1 cells migrating to TDLNs was increased as compared to anti-KLH isotype (FIG. 6B and FIG. 6C), consistent with published reports of systemically active anti-CD40 agonists. See, e.g., Lin et al., J Exp Med. 2020 Aug. 3; 217 (8): e20190673. Resident cDC1 cells in TDLNs showed only modest (p<0.01), if any, increases, in CD86 activation (FIG. 6D), indicating that mCD40×FAP.LP62 activity was restricted to the FAP-expressing tumor. Finally, these changes were associated with increases in the ratio of CD8 T cells to Tregs (CD8: Treg) compared to anti-KLH isotype control 240 hours post-treatment (FIG. 6E).

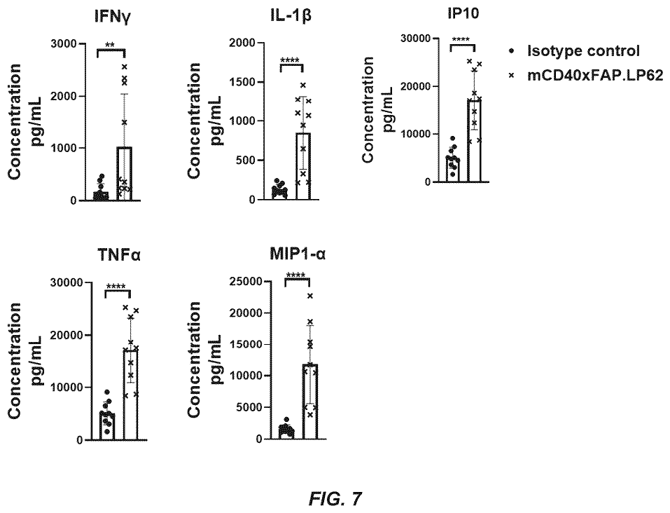

Th1 cytokine levels in KPCY tumors at 240 hours post-treatment with mCD40×FAP.LP62 or mCD40×HEL isotype control (10 mg/kg, QDx1) were also assessed. As shown in FIG. 7, mCD40×FAP.LP62 induced several Th1 cytokines in the tumor, including IL-1β, IP10, TNF-α, IFN-γ, and MIP-1α 240 hours post-treatment as compared to mCD40×HEL isotype control-treated mice (n=10 mice per group across 2 independent experiments for the isotype control and 3 and 10 mice per group for mCD40×FAP.LP62 across two independent experiments).

No significant cytokine induction was observed in the periphery compared to control-treated mice, thus supporting the modality and mechanism of action for localized tumoral activation while sparing systemic CD40 activation (data not shown).

Taken together, these data demonstrate that mCD40×FAP.LP62 activates CD40-mediated innate immune responses in the tumor that engage adaptive immunity leading to efficacy.

### Example 10: Activation of mCD40×FAP.LP62 is Limited to FAP-Positive Tumors in MC38-FAP Syngeneic Mouse Tumor Model

The antitumor effects of mCD40×FAP.LP62 was compared to a systemically active anti-CD40 agonist in the MC38-FAP tumor-bearing mouse model in which every MC38 tumor cell expresses FAP levels comparable to human tumors (data not shown). Tumor volumes following a single dose of mCD40×FAP.LP62 or anti-CD40 as monotherapy were monitored along with alanine aminotransferase and cytokine changes.

C57/BL6 mice were implanted with 5×105 MC38-FAP cells subcutaneously, randomized into treatment groups of 10 mice each when average tumor volumes reached approximately 75 mm³ to 150 mm³, and dosed once via the intraperitoneal (IP) route with either 10 mg/kg mCD40×FAP.LP62, equimolar 8.6 mg/kg anti-CD40 or 30 mg/kg anti-hen egg lysozyme isotype bispecific antibody control, mCD40×HEL. Because this isotype antibody contains the same anti-CD40 antibody arms as mCD40×FAP.LP62, this control enabled the evaluation of FAP binding-dependent CD40 agonist activity in vivo.

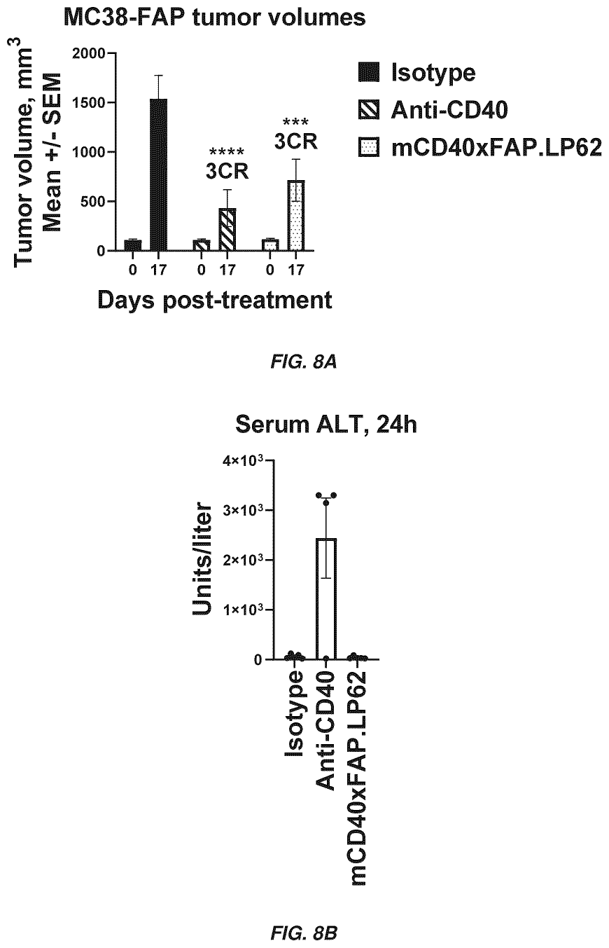

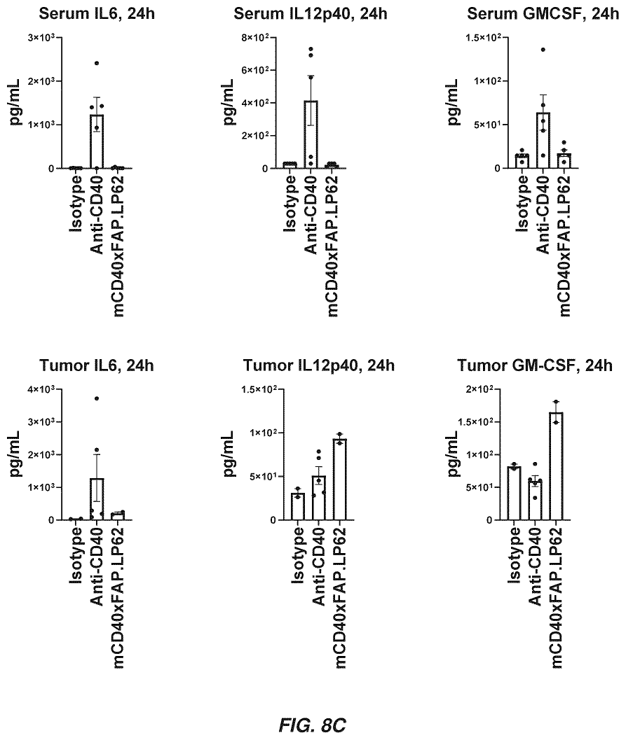

Mean tumor volumes are shown in FIG. 8A. Significant single agent activity of mCD40×FAP.LP62 was observed compared to isotype control and with similar 3 complete responders as the systemically active anti-CD40 agonist (n=10 mice per group within 1 experiment).

Serum levels of the liver enzyme, alanine aminotransferase (ALT), and cytokines downstream of CD40 activation were also quantified at 24 hours post-treatment (QDx1) with 12 mg/kg mCD40×FAP.LP62, equimolar 10.3 mg/kg anti-CD40, or 12 mg/kg mCD40×HEL isotype control in MC38-FAP tumor bearing mice. As shown in FIG. 8B, while ALT levels increased with anti-CD40 treatment, no such change was observed with mCD40×FAP.LP62 or isotype control. Similarly, IL6, IL12p40, and GM-CSF cytokine induction was observed only with anti-CD40 at 24 hours post-treatment (FIG. 8C). In contrast, tumoral induction of these cytokines was observed from mice treated with both anti-CD40 and mCD40×FAP.LP62 compared to isotype control. ALT was quantified from 4-5 mice per group, serum cytokine from 5 mice per group, and tumor cytokine from 2-5 mice per group within 1 experiment.

Taken together, these data demonstrate that not only is mCD40×FAP.LP62 able to restrict CD40 activation to FAP-positive tumors and limit the toxicity observed with systemically active anti-CD40, but also that tumor-restricted CD40 activation is sufficient to drive tumor growth inhibition comparable to that of a systemically active anti-CD40 agonist.

### Example 11: In Vitro Human Tumor Pharmacodynamic Effects

CD40.9H3×FAP.LP62 was evaluated for the ability to activate CD40 in dissociated human patient tumor cells across 5 different donors: 2 pancreatic, 2 non-small cell lung, and 1 squamous lung. Cells were treated with either CD40.9H3×FAP.LP62, an analog of RO7300490, or an anti-HEL×FAP isotype control antibody for 24 hours and cytokine release was quantified from supernatants.

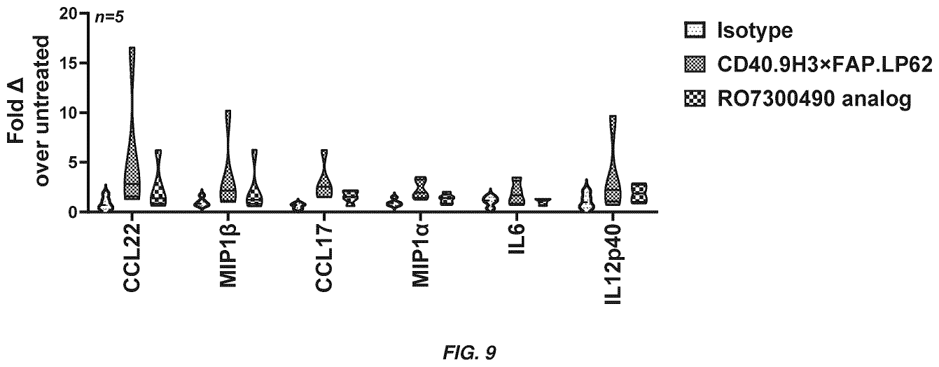

The results are shown in FIG. 9. Cytokines were induced upon treatment with CD40.9H3×FAP.LP62 compared to isotype control. Additionally, CD40.9H3×FAP.LP62 consistently drove higher levels of cytokine release as compared to the analog of RO7300490.

### Example 12: Antibody Internalization of CD40.9H3×FAP.LP62

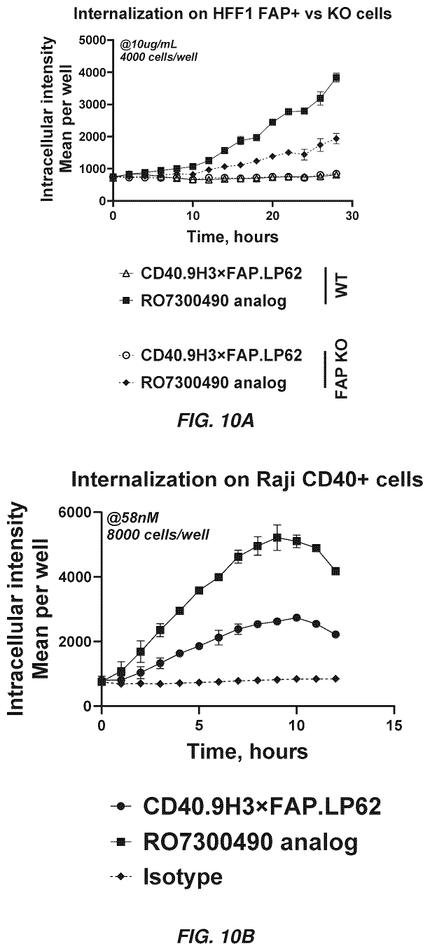

The internalization rate of CD40.9H3×FAP.LP62 and an analog of RO7300490 antibodies upon binding to target was evaluated by conjugation to a pH-sensitive rhodamine fluorogenic dye (pHRodo) that increases fluorescence as the environment becomes more acidic upon intracellular trafficking to lysosomes. FAP-expressing HFF1 and control FAP knockout HFF1 cells were treated with pHRodo-conjugated antibodies and intracellular fluorescence was monitored by live microscopy imaging. Fluorescence in HFF1 cells was similar to HFF1 FAP knockout cells upon treatment with pHRodo-CD40.9H3×FAP.LP62, indicating no FAP-dependent internalization out to 28 hours of imaging (FIG. 10A). In contrast, fluorescence in HFF1 cells upon treatment with pHRodo-RO7300490 increased over time and as compared to control HFF1 FAP knockout treated cells, indicating that the pHRodo-RO7300490 analog is internalized upon binding to FAP. Data shown represent 2 independent experiments.

Internalization was also evaluated using CD40-expressing Raji cells (FIG. 10B). While intracellular fluorescence of pHRodo-CD40.9H3×FAP.LP62-treated Raji cells increased over time as compared to anti-HEL×FAP isotype control antibody, the rate of increase was less than that observed with pHRodo-RO7300490, suggesting the pHRodo-RO7300490 analog is internalized at a faster rate than pHRodo-CD40.9H3×FAP.LP62 upon binding to CD40. Data shown represent 2 independent experiments.

### Example 13: CD40.9H3×FAP.LP62 Activity with Soluble FAP

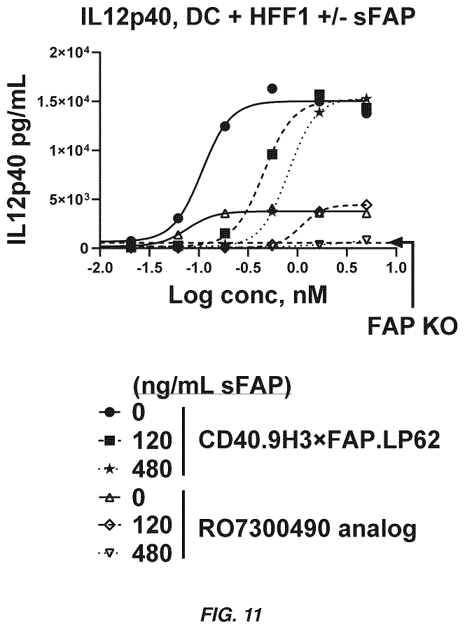

Because soluble FAP can be shed from cells at levels up to 200 ng/mL in circulation in healthy human donors (Oncotarget. 2017 May 2; 8 (18): 30050-30062), the effect of soluble FAP on the activity of CD40.9H3×FAP.LP62 was evaluated compared to an analog of RO7300490. Co-cultures of human DCs and HFF1 cells were treated with either CD40.9H3×FAP.LP62 or RO7300490 analog along with increasing amounts of soluble FAP. CD40.9H3×FAP.LP62 induced dose-dependent secretion of IL12p40 with a 4.2-fold and 7.5-fold reduction in potency with the addition of 120 ng/mL and 480 ng/ml of soluble FAP as compared to the absence of soluble FAP (FIG. 11). No IL12p40 secretion was observed when DCs were co-cultured with HFF1 FAP KO cells (absence of cellular FAP). In contrast, the presence of soluble FAP reduced the potency of the RO7300490 analog by 14.2-fold and 70.9 fold, respectively. Mean (+SD) EC50 concentrations for IL12p40 release are summarized in Table 6 below (n=2 donors within 1 experiment).

TABLE 6
Effect of Soluble FAP on Antibody-induced IL12p40 Release

| | CD40.9H3 × FAP.LP62 | | | RO7300490 analog | | |
|---|---|---|---|---|---|---|
| Soluble FAP ng/mL | 0 | 120 | 480 | 0 | 120 | 480 |
| Mean (±SD) | 0.13 ± 0.03 | 0.46 ± 0.01 | 0.80 ± 0.01 | 0.09 ± 0.02 | 1.14 ± 0.08 | 515.71 ± 721.66 |
| EC50, nM | | | | | | |

### Example 14: CD40.9H3×FAP.LP62 Primes and Activates CD4+ T cells

Because a physiologic function of macrophages is to prime and activate CD4 T cells, the ability of CD40.9H3×FAP.LP62-activated macrophages to induce CD4 T cell function was evaluated and compared to an analog of MP0317 (SEQ ID NO: 38). In a tri-culture assay of macrophages, CD4 T cells, and HFF1 cells, CD40.9H3×FAP.LP62 induced secretion of the T cell-specific cytokine, IL2, in a dose-dependent manner while no IL2 was observed with the anti-HEL×FAP isotype control. See FIG. 12. Additionally, CD40.9H3×FAP.LP62 was more potent in driving IL2 release as compared to the MP0317 analog. Mean (+SD) EC50 concentrations for IL2 secretion are summarized in Table 7 (n=3 donors across 2 independent experiments).

TABLE 7
Antibody-induced T Cell Activation and IL2 Release

| | CD40.9H3 × FAP.LP62 | MP0317 analog |
|---|---|---|
| Mean (±SD) EC50, nM | 0.019 ± 0.01 | 0.19 ± 0.1 |

### Example 15: CD40.9H3×FAP.LP62 Bispecific Antibody Antitumor Activity is Enhanced in Combination with PD-1 Blockade

CD40 activation can bridge innate and adaptive antitumor immune responses. Therefore, the impact of combining CD40.9H3×FAP.LP62 with programmed cell death protein 1 (PD-1) blockade was evaluated in a tri-culture assay of DCs, HFF1 cells, and T cells. HFF1 FAP KO cells were included as a negative control.

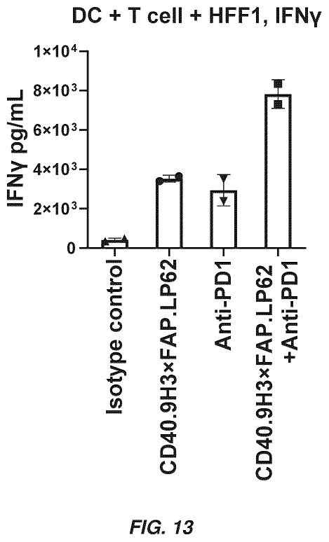

The results are shown in FIG. 13. No IFNγ secretion was observed with the combination of anti-hen egg lysozyme bispecific antibody (anti-HEL×FAP) and anti-diptheria toxin isotype control antibodies for CD40.9H3×FAP.LP62 and anti-PD1 antibody (nivolumab), respectively. While CD40.9H3×FAP.LP62 alone induced IFNγ release that was comparable to levels observed with anti-PD1 antibody alone, the combination of CD40.9H3×FAP.LP62 and anti-PD1 antibody trending further induction of IFNγ compared to either alone (n=3 DC and T cell donor pairs across 2 independent experiments).

The combination of anti-CD40/FAP antibody and PD-1 blockade was also assessed in vivo using the KPCY mouse tumor model described above. The KPCY mouse tumor model is only moderately responsive to immune checkpoint blockade (such as anti-mCTLA4 and anti-mPD1 antibodies). See, e.g., Li et al. Immunity. 2018 Jul. 17; 49 (1): 178-193.c7. Therefore, the ability of mCD40×FAP.LP62 to enhance tumor growth inhibition in combination with anti-mPD1 blocking antibody was evaluated.

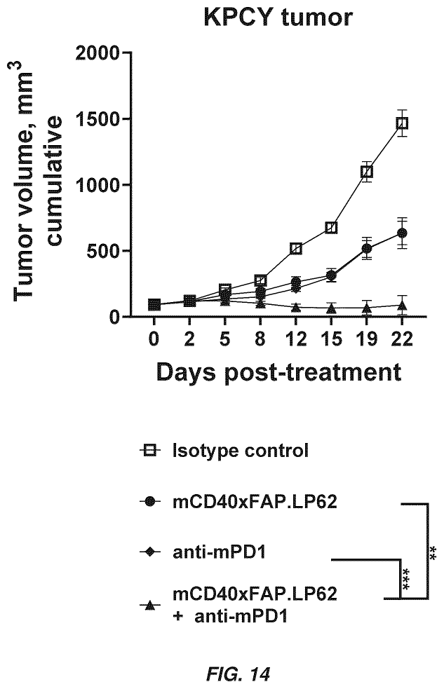

The results are shown in FIG. 14. While no tumor-free mice were observed upon treatment with mCD40×FAP.LP62 alone daily (3 mg/kg) or anti-mPD1 antibody alone every three weeks (10 mg/kg), the combination significantly improved tumor regression, with 8 out of 10 tumor-free mice at Day 22 post-treatment (n=10 mice per group across 2 independent experiments).

### Example 16: Nonclinical Pharmacokinetics and Toxicology of CD40.9H3×FAP.LP62

The pharmacokinetic (PK)/toxicokinetic (TK) of CD40.9H3×FAP.LP62 was investigated in cynomolgus monkey following a single intravenous (IV) dose (at 3 mg/kg in study DT23032 and at 0.3, 6, or 20 mg/kg in study DT23026) or repeated IV dosing (at 30 mg/kg given once weekly for 4 weeks in study DN23134, and at 30 mg/kg and 80 mg/kg weekly, or 80 mg/kg twice weekly in study DN23160). CD40.9H3×FAP.LP62 demonstrated nonlinearity in PK reflected in the half-life and clearance, which is consistent with nonlinear target-mediated drug disposition (TMDD). Anti-drug antibody (ADA) formation was observed in all studies as early as 1 week after dosing and likely contributed to decreased exposures after repeated dosing of the corresponding subjects.

The pivotal 1-month toxicity study in monkeys demonstrated that CD40.9H3×FAP.LP62 has an acceptable safety profile that can support the proposed FIH study in patients with advanced cancer. In brief, CD40.9H3×FAP.LP62 was administered intravenously to monkeys at 30 or 80 mg/kg/dose weekly (QW) (5 doses total) or 80 mg/kg/dose twice weekly (BIW) (9 doses total) for 1 month. CD40.9H3×FAP.LP62 was clinically well tolerated and showed target engagement and pharmacodynamic effects at all doses. There were no significant CD40.9H3×FAP.LP62-related clinical signs, adverse changes in clinical pathology or immunological parameters, or effects on safety pharmacology assessments at any dose tested. Anatomopathologically, inflammatory changes were noted at all doses and included 1) perivascular lymphoplasmacytic or lymphohistiocytic inflammation in the kidney, liver, pancreas, submandibular salivary gland, urinary bladder, epididymis, prostate, uterus, cervix, and vagina; 2) tubular degeneration/mixed cell inflammation with regeneration in the kidney; and 3) increases in size, weight, and/or cellularity of lymphoid tissues/organs. None of the inflammatory changes were considered adverse (due to minimal to mild severity and/or lacking clinical pathology correlates), except for the moderate perivascular inflammation in the reproductive tract of one female at 30 mg/kg QW. Additional findings in this study included increased complement activation product C3a at ≥30 mg/kg/week on Day 8 and at ≥80 mg/kg/week on Days 15 and/or 30; these were considered nonadverse due to the absence of clinical signs or changes in total complement CH50. Accordingly, the no-observed-adverse-effect level (NOAEL; identified in males only) and the highest non-severely toxic dose (HNSTD; identified in both sexes) were considered to be 80 mg/kg BIW (mean sex-combined AUC[0-168h] 587,000 μg·h/mL in Week 4).

### Example 17: Phase 1/1b Clinical Trial of CD40.9H3×FAP.LP62 as Monotherapy and Combination Therapy in Participants with Advanced Solid Malignancies

### Study Design—Overall Design

This is a Phase 1, multi-center, open-label, first-in-human study to assess the safety, tolerability, pharmacokinetics (PK), pharmacodynamics (PD), and preliminary anti-tumor activity of CD40.9H3×FAP.LP62 administered as monotherapy and in combination with nivolumab, including with or without standard of care chemotherapy (FOLFOX or CAPOX) in participants with select advanced/metastatic solid tumors including non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN). Administration of CD40.9H3×FAP.LP62 as a monotherapy is investigated using either an intravenous route of administration or a subcutaneous route of administration.

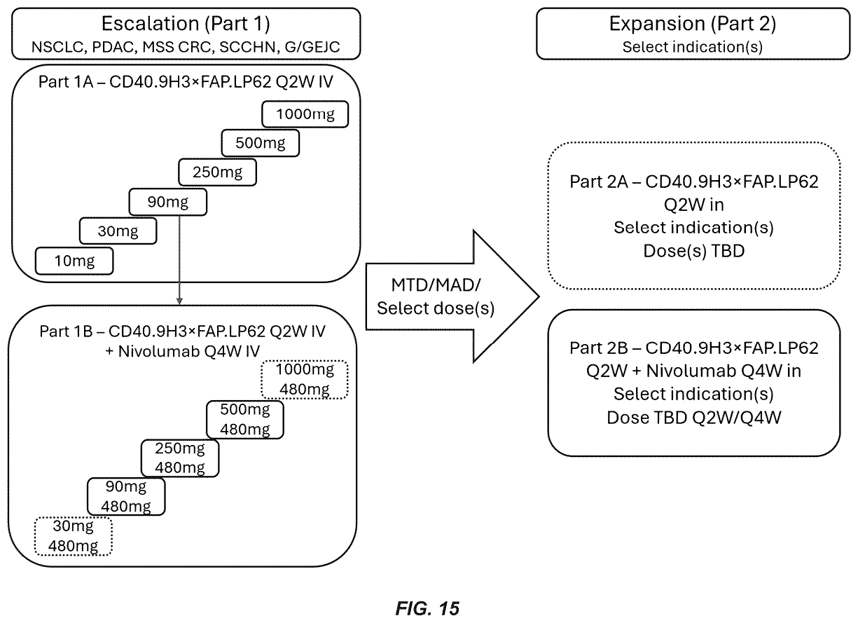

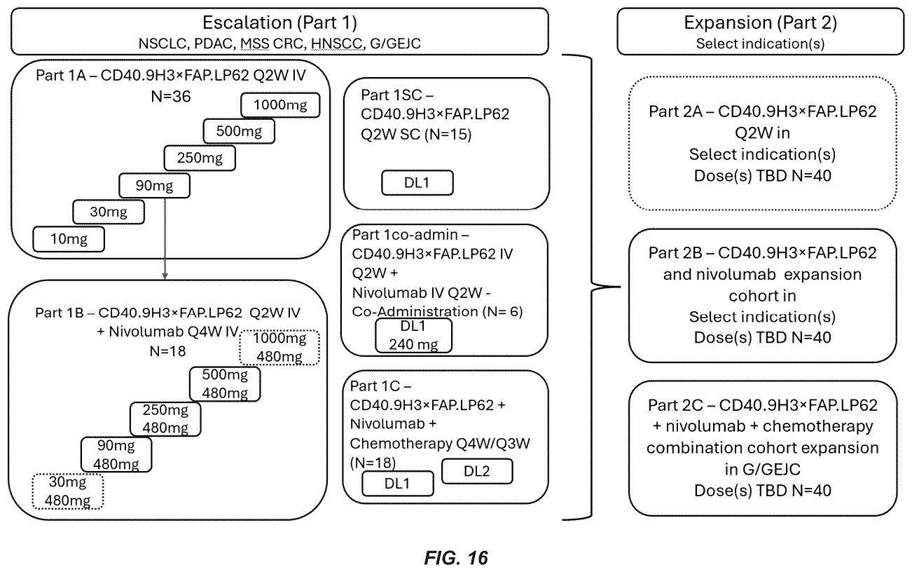

The study design is presented in FIG. 15. An alternative study design, including details regarding testing additional routes of administration and combination arms that include chemotherapy, is presented in FIG. 16.

### Treatment Period

The participant begins treatment after confirmation of eligibility. The treatment period lasts for up to 2 years from the first dose of study intervention. To evaluate safety, weekly study visits are performed for the first 6 weeks after the first dose of study intervention, followed by study visits Q2W thereafter. Imaging for tumor assessment occurs every 8 weeks from the date of first dose during the first 48 weeks, followed by every 12 weeks until disease progression or treatment discontinuation, whichever occurs later. During the treatment period for all study parts, additional study visits to collect samples for intensive PK, anti-drug antibody (ADA), and biomarker assessments are required.

Table 8A shows the on-treatment Schedule of Activities for intravenous sequential administration of CD40.9H3×FAP.LP62 with or without nivolumab, for subcutaneous administration of CD40.9H3×FAP.LP62, and for intravenous co-administration of CD40.9H3×FAP.LP62 and nivolumab; Table 8B shows the on-treatment Schedule of Activities for co-administration of CD40.9H3×FAP.LP62, nivolumab, and chemotherapeutic regimen CAPOX; Table 8C shows the on-treatment Schedule of Activities for co-administration of CD40.9H3×FAP.LP62, nivolumab, and chemotherapeutic regimen FOLFOX; and FIG. 15 or FIG. 16 shows dose selection and timing.

TABLE 8A
On-Treatment - Schedule of Activities for All Study Parts (except Part 1C)

| | Cycle 1 | | | | Cycle 2 | | | Cycle 3 and Beyond | | |
|---|---|---|---|---|---|---|---|---|---|---|
| | (28-day Cycle) | | | | (28-day Cycle) | | | (28-day Cycles) | | |
| | | D | D | D | D | D | D | D | D | |
| | | 8 ±2 | 15 ±2 | 22 ±2 | 1 ±2 | 8 ±2 | 15 ±2 | 1 ±2 | 15 ±2 | |
| Procedure | D 1 | Days | Days | Days | Days | Days | Days | Days | Days | Notes |

Study Intervention - Intravenous Sequential

| | | | | | | | |
|---|---|---|---|---|---|---|---|
| CD40.9H3 × | X | X | X | X | X | X | |
| FAP.LP62 | | | | | | | |
| Administration | | | | | | | |
| Q2W (all parts | | | | | | | |
| using | | | | | | | |
| intravenous | | | | | | | |
| administration) | | | | | | | |
| Nivolumab | X | | X | | X | | In |
| Administration | | | | | | | combination |
| Q4W (Part 1B/ | | | | | | | parts, |
| Part 2B) | | | | | | | administer |
| | | | | | | | nivolumab |
| | | | | | | | before |
| | | | | | | | CD40.9H3 × |
| | | | | | | | FAP.LP62 |

Study Intervention - Subcutaneous

| | | | | | | |
|---|---|---|---|---|---|---|
| CD40.9H3 × | X | X | X | X | X | X |
| FAP.LP62 | | | | | | |
| Administration | | | | | | |

Study Intervention - Intravenous Co-administration

| | | | | | | |
|---|---|---|---|---|---|---|
| CD40.9H3 × | X | X | X | X | X | X |
| FAP.LP62 | | | | | | |
| and Nivolumab | | | | | | |
| Co- | | | | | | |
| Administration | | | | | | |

TABLE 8B
On-Treatment - Schedule of Activities for Part 1C: CAPOX (Q3W) Schedule

| | Cycle 1 | | | Cycle 2 | | Cycle 3 and Beyond | |
|---|---|---|---|---|---|---|---|
| | (21-day Cycle) | | | (21-day Cycle) | | (21-day Cycles) | |
| | | D | D | D | D | D | |
| | | 8 ±2 | 15 ±2 | 1 ±2 | 8 ±2 | 1 ±2 | |
| Procedure | D 1 | Days | Days | Days | Days | Days | Notes |

Study Intervention - Intravenous Sequential

| | | | | |
|---|---|---|---|---|
| CD40.9H3 × | X | X | X | |
| FAP.LP62 | | | | |
| Administration | | | | |
| Q3W | | | | |
| Nivolumab | X | X | X | |
| Administration | | | | |
| Q3W | | | | |
| Chemotherapy- | X | X | X | Administer |
| Oxaliplatin | | | | Nivolumab first, |
| Capecitabine | | | | then CD40.9H3 × |
| | | | | FAP.LP62, and then |
| | | | | chemotherapy |
| | | | | Capecitabine PO BID |
| | | | | Day 1-Day 14 |

TABLE 8C
On-Treatment - Schedule of Activities for Part 1C: FOLFOX (Q3W) Schedule

| | Cycle 1 | | | | Cycle 2 | | Cycle 3 and Beyond | | |
|---|---|---|---|---|---|---|---|---|---|
| | (28-day Cycle) | | | | (28-day Cycle) | | (28-day Cycles) | | |
| | | D | D | D | D | D | D | D | |
| | | 8 ±2 | 15 ±2 | 22 ±2 | 1 ±2 | 15 ±2 | 1 ±2 | 15 ±2 | |
| Procedure | D 1 | Days | Days | Days | Days | Days | Days | Days | Notes |

Study Intervention - Intravenous Sequential

| | | | | | | | |
|---|---|---|---|---|---|---|---|
| CD40.9H3 × | X | X | X | X | X | X | Administer after |
| FAP.LP62 | | | | | | | nivolumab |
| Administration | | | | | | | |
| Q2W | | | | | | | |
| Nivolumab | X | X | X | X | X | X | Administer |
| Administration | | | | | | | nivolumab before |
| Q2W | | | | | | | CD40.9H3 × |
| | | | | | | | FAP.LP62 |
| Chemotherapy- | X | X | X | X | X | X | Administer |
| Oxaliplatin | | | | | | | Nivolumab first, |
| Folinic acid (or | | | | | | | then |
| therapeutic | | | | | | | CD40.9H3 × |
| equivalent) | | | | | | | FAP.LP62 and then |
| Fluorouracil | | | | | | | chemotherapy |

### Study Interventions

### CD40.9H3×FAP.LP62

Participants receive CD40.9H3×FAP.LP62 infusion over approximately 30 minutes on Day 1 and Day 15 of each 28-day treatment cycle until progression, unacceptable toxicity, withdrawal of consent, completion of 2 years of treatment, or the study ends, whichever occurs first. If needed, the intravenous (IV) line is flushed with an appropriate amount of diluent (e.g., 0.9% sodium chloride) to ensure that the complete dose is administered over the allotted infusion time. Study intervention begin within 3 calendar days of treatment assignment.

Doses of study intervention(s) may be interrupted, delayed, or discontinued depending on how well the participant tolerates the treatment. Day 1 dosing visits are not skipped, only delayed; Day 15 dosing visit may be skipped if not administered within protocol-allowed windows. Intra-participant dose escalation may be allowed.

TABLE 9
Study Interventions Administered

| Type/Intervention | Unit Dose | |
|---|---|---|
| Name/Dose Formulation | Strength(s) | |
| Drug/ | 50 | mg/mL |
| CD40.9H3 × FAP.LP6 | | |
| 2/Concentrate for | | |
| solution for infusion | | |
| Drug/Nivolumab/ | 10 | mg/mL |
| Concentrate for | | |
| solution for infusion | | |
| Drug/Oxaliplatin/ | 5 | mg/mL |
| Concentrate for | | |
| Solution for infusion | | |
| Drug/Capecitabine/Tablets | 150 | mg |
| | 500 | mg |
| Drug/Fluorouracil/ | 50 | mg/mL |
| Solution for Injection | | |
| Drug/Calcium | 10 | mg/mL |
| folinate/Solution for | | |
| Injection | | |

TABLE 10
Study Arms

| Arm Title/ | Intervention Description/ | Route of Administration/ |
|---|---|---|
| Arm Use | Dosage Levels | Administration Time |
| Part 1A and Part 2A: | Various doses on Day 1 and Day | IV infusion over 30 minutes |
| CD40.9H3 × FAP.LP62 | 15 of each cycle | |
| Part 1B and Part 2B: | Nivolumab 480 mg on day 1 + | Nivolumab IV infusion over 30 |
| Nivolumab + | CD40.9H3 × FAP.LP62 various | minutes CD40.9H3 × FAP.LP62 IV |
| CD40.9H3 × FAP.LP62 | doses on Day 1 and Day 15 of each | infusion over 30 minutes |
| | cycle | |
| Part 1C: Nivolumab + | Nivolumab 240 mg on day 1 and | Nivolumab IV infusion over 30 |
| CD40.9H3 × FAP.LP62 + | day 15 + | minutes |
| FOLFOX | CD40.9H3 × FAP.LP62 select doses | CD40.9H3 × FAP.LP62 IV infusion |
| | on day 1 and day 15 + | over 30 minutes |
| | Oxaliplatin 85 mg/m² on day 1 and | Oxaliplatin, Folinic Acid and |
| | day 15 + | Fluorouracil as per institutional |
| | Folinic Acid 400 mg/m² on day 1 | practice |
| | and day 15 + | |
| | Fluorouracil 400 mg/m² bolus on 1 | |
| | and day 15 and 1200 mg/m² | |
| | continuous infusion on day 1/2 and | |
| | day 15/16 of each 28 day cycle | |
| Part 1C: Nivolumab + | Nivolumab 360 mg on day 1 + | Nivolumab IV infusion over 30 |
| CD40.9H3 × FAP.LP62 + | CD40.9H3 × FAP.LP62 select doses | minutes |
| CAPOX | on day 1 + | CD40.9H3 × FAP.LP62 IV infusion |
| | Oxaliplatin 130 mg/m² on day 1 + | over 30 minutes |
| | Capecitabine 1000 mg/m² PO BID | Oxaliplatin, and Capecitabine as |
| | day 1 to day 14 of each 21 day | per institutional practice |
| | cycle | |
| Part 1 co-admin and | Nivolumab 240 mg + | Nivolumab + CD40.9H3 × FAP.LP62 |
| 2B: Nivolumab + | CD40.9H3 × FAP.LP62 select doses | IV Co-administration over 30 |
| CD40.9H3 × FAP.LP62 | on Day 1 and Day 15 of each 28 | minutes |
| | cycle | |
| Part 1SC: | Select dose(s) on Day 1 and Day | Subcutaneous injection |
| CD40.9H3 × FAP.LP62 | 15 of each 28 day cycle | |
| Part 2C | Combination expansion based on | Based on select route and regimen |
| | select dose(s) and schedule from | |
| | Part 1C | |

Abbreviation: BID, twice daily; IV, intravenous; PO, oral.

### Nivolumab

Nivolumab (also referred to as BMS-936558, MDX1106, or ONO-4538) is a human monoclonal antibody (HuMAb; immunoglobulin G4 [IgG4]-S228P) that targets the programmed death-1 (PD-1) cluster of differentiation 279 (CD279) cell surface membrane receptor. PD-1 is a negative regulatory molecule expressed by activated T and B lymphocytes. Binding of PD-1 to its ligands, programmed death-ligands 1 (PD-L1) and 2 (PD-L2), results in the down-regulation of lymphocyte activation. Inhibition of the interaction between PD-1 and its ligands promotes immune responses and antigen-specific T-cell responses to both foreign antigens as well as self-antigens. Nivolumab is expressed in Chinese hamster ovary (CHO) cells and is produced using standard mammalian cell cultivation and chromatographic purification technologies. The clinical study product is a sterile solution for parenteral administration.

Nivolumab (OPDIVO™) is approved for the treatment of several types of cancer in multiple regions including the United States (US, December-2014), the European Union (EU, June-2015), and Japan (July-2014). Nivolumab has demonstrated durable responses exceeding 6 months as monotherapy in several tumor types, including NSCLC, melanoma, RCC, cHL, SCLC, gastric cancer, SCCHN, urothelial cancer, HCC, and CRC. In confirmatory trials, nivolumab as monotherapy demonstrated a statistically significant improvement in OS as compared with the current standard of care in patients with advanced or metastatic NSCLC, unresectable or metastatic melanoma, advanced RCC, or recurrent or metastatic SCCHN. Details of the clinical activity in these various malignancies are provided in the USPI and SmPC.

Participants receive nivolumab at a dose of 480 mg in Part 1B and/or Part 2B over an approximately 30-minute infusion each treatment cycle until progression, unacceptable toxicity, withdrawal of consent, completion of 2 years of treatment, or the study ends, whichever occurs first. If needed, the intravenous (IV) line is flushed with an appropriate amount of diluent (e.g., 0.9% sodium chloride or 5% dextrose in water) to ensure that the complete dose is administered over approximately 30 minutes. Study intervention begins within 3 calendar days of treatment assignment.

When nivolumab and CD40.9H3×FAP.LP62 are to be administered on the same day, nivolumab is administered first. Nivolumab infusion is promptly followed by a flush of diluent to clear the line of nivolumab before starting the CD40.9H3×FAP.LP62 infusion. The second infusion is CD40.9H3×FAP.LP62 and starts after the infusion line has been flushed, filters changed, and the participant has been observed to ensure no infusion reaction has occurred beginning at least 30 minutes after completion of the infusion of nivolumab.

Pre-medications are not recommended for the first dose of nivolumab.

Separate infusion bags and filters are used when administering nivolumab and CD40.9H3×FAP.LP62 on the same day.

### Part 1 Dose Escalation Cohorts

Up to approximately 54 participants are treated in Part 1, including approximately 36 participants in Part 1A Monotherapy Dose Escalation and approximately 18 participants in Part 1B Combination Dose Escalation. The sample sizes for the study are determined based on the number of anticipated dose levels and the number of participants in each dose level.

Specifically, approximately 36 participants across 6 dose levels with at least 3 DLT-evaluable participants per dose level are included for Part 1A and approximately 18 patients across 3 dose levels with at least 3 DLT-evaluable participants per dose level are included in Part 1B. In Part 1, a maximum of approximately 12 patients in total may be treated at specific dose level(s) and schedule(s).

Part 1 evaluates the safety and tolerability of CD40.9H3×FAP.LP62 monotherapy (Part 1A) and combination with nivolumab (Part 1B) based on DLTs, using the Bayesian optimal interval (BOIN) design to guide escalation decisions and the overall assessment of available safety, PK, and pharmacodynamic data. Part I also evaluates the safety and tolerability of CD40.9H3×FAP.LP62 in combination with nivolumab in combination with standard of care chemotherapy (FOLFAX or CAPOX) using the same design and overall assessment. The dose escalation and de-escalation in the BOIN design is determined by comparing the observed DLT rate at the current dose with a pair of fixed dose escalation and de-escalation boundaries. This allows generation of a decision table that guides dose selection depending on the number of participants' treated and observed DLTs.

In monotherapy or in combination therapy, additional participants may be enrolled at any dose level previously established to be tolerable (during the DLT evaluation) to obtain additional experience (hereon referred to as “backfill”). Backfill may occur in parallel with additional dose exploration at higher doses. Backfill participants for additional assessments are considered as nonDLT; however, PK, pharmacodynamics, and safety data obtained from these participants contributes to the totality of the data to guide dose selection.

NSCLC, MSS CRC, SCCHN, G/GEJC, and PDAC were selected as the indications for inclusion in this dose escalation study, based on high prevalence of FAP expression by immunohistochemistry (IHC) as well as co-expression of FAP with resident dendritic cell gene signatures.

A starting dose of 10 mg IV Q2W is used for this FIH study. This was determined based on the totality of the data from the pivotal toxicology study, external data from another CD40×FAP bispecific antibody, and PK/PD modeling.

As described in Example 16, the pivotal 1-month toxicity study in monkeys demonstrated that CD40.9H3×FAP.LP62 has a favorable safety profile with the highest non-severely toxic dose (HNSTD) of 80 mg/kg twice weekly (BIW), the highest dose evaluated in the study. This supports a maximum recommended starting dose (MRSD) of 3200 mg Q2W, which is higher than the current proposed FIH dose range of 10 to 1000 mg. Additionally, RO7300490, a CD40×FAP bispecific mAb in the same format as CD40.9H3×FAP.LP62, has been shown to be well-tolerated across the investigated dose range of 16 to 1100 mg IV with no dose-limiting toxicities (DLTs) observed or maximal tolerable dose (MTD) determined (Melero et al. Journal for ImmunoTherapy of Cancer, 2023; 11).

Given the immune-stimulating mechanism of action (MoA), the starting dose is based on the pharmacological activity of CD40.9H3×FAP.LP62 as determined using a tumor growth inhibition (TGI) model that was developed using preclinical PK/PD data. This model was used to estimate human tumor growth following Q2W treatment and predicted a dose of 10 mg would reduce tumor growth by 50% (ED50) at 50 days following the start of treatment. Notably, the projected Cmax (4.88 μg/mL) and AUC0-336h (76.3 μg·h/mL) in humans at 10 mg are at least 1000-fold below the HNSTD exposures in monkeys from the 1-month toxicity study (as described in Example 16).

The TGI model predicted that responses will begin to plateau at 80 mg IV Q2W and reach a maximal effect between 240 to 360 mg IV Q2W. Higher dose levels are included in order to assess a potential non-linear relationship between exposure and response.

Fixed dosing (vs weight-based) was chosen for this clinical study due to the case of preparation, a reduced risk of medication errors, and the observation that for most biologics the 2 dosing approaches perform similarly (Erstad BL and Davis LE. Ann Pharmacother, 2024; 58:91-5; Hendrikx et al. Oncologist, 2017; 22:1212-21).

The nivolumab dose of 480 mg IV Q4W was selected based on clinical data and modelling and simulation approaches using population PK (PPK) and exposure-response analyses of data from studies in multiple tumor types (e.g., melanoma, NSCLC, and RCC) where body weight normalized dosing (mg/kg) was used.

### Part 1A CD40.9H3×FAP.LP62 Monotherapy Dose Escalation

Part 1A has 6 planned dose levels of CD40.9H3×FAP.LP62: 10 mg, 30 mg, 90 mg, 250 mg, 500 mg, and 1000 mg administered IV Q2W (FIG. 15, Part 1A). If a planned dose level is associated with an unacceptable frequency of toxicities, then an intermediate dose may be evaluated. After preliminary evaluation of safety and PK data from these intermediate doses, re-escalating doses of CD40.9H3×FAP.LP62 may be initiated.

### Part 1B CD40.9H3×FAP.LP62 Dose Escalation+Nivolumab

Part 1B evaluates the safety and tolerability of various doses of CD40.9H3×FAP.LP62 in combination with nivolumab (480 mg IV Q4W). There are 3 planned dose levels of CD40.9H3×FAP.LP62: 90 mg, 250 mg, and 500 mg administered IV Q2W (FIG. 15, Part 1B). Additional doses of CD40.9H3×FAP.LP62 may be evaluated, if warranted, based on emerging data. Treatment in Part 1B is initiated in a staggered manner relative to Part 1A, such that Part 1B can be initiated in parallel with the Part 1A dose escalation. Part 1B evaluates CD40.9H3×FAP.LP62 doses that have been cleared for safety in Part 1A. At no point does the dose of CD40.9H3×FAP.LP62 administered in combination with nivolumab in Part 1B exceed the highest dose determined to be tolerable in Part 1A. In Part 1B, the dose of CD40.9H3×FAP.LP62 is escalated, whereas the dose of nivolumab is fixed at 480 mg Q4W. However, if it appears that a planned dose level is associated with an unacceptable frequency of toxicities, then an intermediate dose level or de-escalating doses may be evaluated, and potential re-escalating doses of CD40.9H3×FAP.LP62 may then be initiated.

### Part 1C CD40.9H3×FAP.LP62+Nivolumab+FOLFOX/CAPOX Dose Escalation

Part 1C evaluates the safety and tolerability of selected doses of CD40.9H3×FAP.LP62 in combination with nivolumab and standard of care chemotherapy in GC/GEJ adenocarcinoma (FIG. 16, Part 1C). CAPOX is administered Q3W. FOLFOX is administered Q2W. There are two planned dose levels of CD40.9H3×FAP.LP62 for both Q2W and Q3W dose regimens selected based on totality of the data from Part 1A and Part 1B. Nivolumab is administered at 240 mg Q2W when in combination with FOLFAX or 360 mg Q3W when in combination with CAPOX.

CD40.9H3×FAP.LP62 and nivolumab is administered with nivolumab administered intravenously first and then CD40.9H3×FAP.LP62 administered second intravenously. Alternatively, both CD40.9H3×FAP.LP62 and nivolumab are administered in the same infusion bag intravenously.

Administration of CAPOX or FOLFOX occurs after administration of CD40.9H3×FAP.LP62. CAPOX is intravenous administration of 130 mg/m² oxaliplatin on Day 1 of each treatment cycle every 3 weeks, and oral administration of 1000 mg/m² capecitabine 1000 mg/m² twice daily (i.e., 1000 mg/m² in the morning and 1000 mg/m² in the evening) on Days 1 to 14 of each treatment cycle every 3 weeks. FOLFOX is intravenous administration of 85 mg/m² oxaliplatin, 400 mg/m² folinic Acid (or therapeutic equivalent), and 400 mg/m² fluorouracil every 2 weeks, and intravenous continuous infusion of 1200 mg/m² fluorouracil over 24 hours daily or per local standard on Day 1 and 2 as well as day 15 and 16 of each 28-day treatment cycle.

### Part 1 CD40.9H3×FAP.LP62+Nivolumab Co-Administration (Part 1 Co-Admin)

Part 1 co-administration (FIG. 16, Part 1 co-admin) evaluates the safety and tolerability of selected dose of CD40.9H3×FAP.LP62 (Q2W) co-administered with Nivolumab 240 mg (Q2W). There is one planned dose of CD40.9H3×FAP.LP62 in combination with Nivolumab co-administrated selected based on the totality of available data including safety, PK, and PD from the doses evaluated in Part 1B and deemed to be tolerable when administered sequentially. CD40.9H3×FAP.LP62 is combined in the same infusion bag as 240 mg of Nivolumab and administered intravenously.

### Part 1 CD40.9H3×FAP.LP62 Monotherapy Subcutaneous Administration (Part 1SC)

Part 1SC evaluates the safety and tolerability of CD40.9H3×FAP.LP62 administered subcutaneously Q2W (FIG. 16, Part 1SC). CD40.9H3×FAP.LP62 is administered subcutaneously in the abdomen. For participants who may require injection at other site(s), doses are split and administered across more than one site; and if this is the case, the split injections are preferably kept close.

### Dose Escalation Decisions for Part 1A Monotherapy, Part 1B Nivolumab Combination, and Part 1C Nivolumab+Chemotherapy Combination

The Part 1A, Part 1B, and Part 1C decisions for escalation, de-escalation, or continuing evaluation at the same dose level is guided by the BOIN escalation design with a target DLT rate of 30% and with an escalation boundary of 23.6% and de-escalation boundary of 35.9%. Approximately 36, 18, and 18 DLT-evaluable participants are treated in Part 1A, Part 1B, and Part 1C, respectively, depending on the number of dose levels and the number of observed DLTs. Dose escalation/de-escalation decisions or the decision to continue enrollment at the current dose are made in consideration of all available safety, PK, and pharmacodynamic (PD) data. Participants enroll in cohorts of 3 to 4 initially. After the first 3 to 4 participants are evaluated, additional participants may be enrolled, as needed. Up to approximately 12 patients total may be treated at specific dose level(s) and schedule(s) in Part 1.

### Treatment Beyond Progression

Accumulating evidence indicates a minority of participants treated with immunotherapy may derive clinical benefit despite initial evidence of PD. Tumor progression and response endpoints are assessed using RECIST v1.1. Treatment beyond progression is allowed in selected participants with initial RECIST v1.1-defined PD, if the benefit/risk assessment favors continued administration of study intervention. Participants are permitted to continue treatment beyond initial PD, up to a maximum of 24 months from date of first dose.

### Inclusion Criteria

Participants are eligible to be included in the study only if all of the following criteria apply:

### Type of Participant and Target Disease Characteristics

(A) Part 1A and Part 1B: Participants with histologically or cytologically confirmed locally advanced unresectable, metastatic, or recurrent malignant tumors including non-small cell lung cancer (NSCLC), pancreatic ductal adenocarcinoma (PDAC), gastric/gastroesophageal junction adenocarcinoma (G/GEJC), microsatellite stable colorectal carcinoma (MSS CRC), and squamous cell carcinoma of the head and neck (SCCHN). Indication-specific criteria are detailed below in Sections (B) to (F) below.
(B) Participants with NSCLC:
   (i) Must have received and then progressed on, or been intolerant to, platinum doublet-based chemotherapy or at least 2 prior lines of systemic therapy for advanced or metastatic disease, OR must have recurrent or progressive disease within 6 months after completing platinum-based chemotherapy for local disease.
   (ii) Participants in Part 1B and Part 2B must have received treatment with anti-PD-(L) 1 therapy.
   (iii) Status of actionable mutations (e.g., EGFR, ALK, ROS1, RET, etc.) must be known and documented; participants with actionable mutations must have received and progressed on, have been intolerant to, or not be a candidate for, standard tyrosine kinase inhibitors.
(C) Participants with SCCHN (oral cavity, pharynx, or larynx):
   (i) Must have received and then progressed on, or been intolerant to, a platinum-containing regimen.
   (ii) Participants in Part 1B and Part 2B must have received treatment with anti-PD-(L) 1 therapy.
   (iii) Historical human papillomavirus (HPV) status for oropharyngeal cancers must be documented, if available. HPV status should have been determined using p16 immunohistochemistry (IHC) or HPV polymerase chain reaction (PCR).
(D) Participants with PDAC:
   (i) Must have received and then progressed on, or been intolerant to (or not be a candidate for) at least 1 prior standard chemotherapy.
(E) Participants with G/GEJC:
   (i) Must have received and then progressed on, or been intolerant to at least 1 standard treatment regimen in the advanced or metastatic setting or have progressed within 6 months of adjuvant therapy.
   (ii) Participants in Part 1B and Part 2B must have received treatment with anti-PD-(L) 1 therapy.
   (iii) Participants with known human epidermal growth factor receptor 2 (HER2)-positive gastric cancer must have received prior treatment with a HER2 inhibitor (e.g., trastuzumab).
   (iv) If available, microsatellite instability (MSI) status or mismatch repair (MMR) status should be documented.
(F) Participants with MSS CRC:
   (i) Must have received and then progressed on or after, or have been intolerant to at least 1 standard systemic therapy for metastatic and/or unresectable disease (or have progressed within 6 months of adjuvant therapy) or have been intolerant to treatment with fluoropyrimidine, oxaliplatin, and irinotecan given as a single regiment or over multiple regiments for metastatic and/or unresectable disease.
   (ii) Prior treatment with fluoropyrimidine, oxaliplatin, and irinotecan given as a single regimen or over multiple regimens is required.
   (iii) Participants must have known MSI status or mismatch repair (MMR) status. If the MSI molecular test and MMR IHC test results are both available, then both MSS and MMR proficiency will be required for study entry. Patients with MSI-high, MSI-low, or MMR deficiency will not be eligible.
   (iv) KRAS, NRAS, and BRAF status, if known, should be documented. If RAS wild-type, prior treatment with an anti-EGFR therapy (e.g., cetuximab or panitumumab) is required.
(G) Part 2A and Part 2B: Participants with histologically or cytologically confirmed locally advanced unresectable, metastatic, or recurrent malignant tumor selected from eligible tumor types in Part 1.
(H) Participants in all parts must have measurable disease by Response Evaluation Criteria in Solid Tumors version 1.1 (RECIST v1.1).
(I) Participants must have an Eastern Cooperative Oncology Group Performance Status of 0 or 1.
(J) Parts 1C and 2C:
   i) All participants have inoperable, advanced, or metastatic GC or GEJ or distal esophageal carcinoma and have histologically confirmed predominant adenocarcinoma. The documentation of GEJ involvement can include biopsy, endoscopy, or imaging.
   ii) Participants have not received previous systemic treatment (including HER2 inhibitors) given as primary therapy for advanced or metastatic disease.
   iii) Prior adjuvant or neoadjuvant chemotherapy, radiotherapy, and/or chemoradiotherapy for GC or GEJ cancer are permitted as long as the last administration of the last regimen (whichever was given last) occurred at least 6 months prior to enrollment.
   iv) All participants must have an evaluable PD-L1 CPS classification (≥5 or <5, or indeterminate).

### Age of Participant

Participant must be ≥18 or the legal age of consent in the jurisdiction in which the study is taking place at the time of signing the ICF.

### Efficacy Assessments

Efficacy assessments for the anti-tumor activity of CD40.9H3×FAP.LP62, alone and in combination with nivolumab, are based on tumor measurements using RECIST v1.1, with computed tomography (CT) and/or magnetic resonance imaging (MRI), as appropriate, at baseline and during the treatment period.

Primary endpoints are as described in Table 11 below:

TABLE 11
Primary Endpoint(s)

| Primary Endpoint | Description of Analysis | Time Frame |
|---|---|---|
| Incidences of AEs, | DLT rate by dose level/schedule, frequency and | For DLTs, up to |
| SAEs, AEs meeting | distribution of treated participants with AEs, using | 28 days |
| protocol-defined DLT | the worst CTC grade according to NCI CTCAE v5.0. | Up to 100 days |
| criteria, AEs leading to | Participants will only be counted (1) once at the PT | after the last |
| discontinuation, and | level, (2) once at the system organ class level, and (3) | dose of study |
| death | once in the “total participant” row at their worst CTC | intervention |
| | grade, regardless of system organ class or preferred | |
| | term (PT). | |

Abbreviations: AE, adverse event; CTC, Common Terminology Criteria; CTCAE, Common Terminology Criteria for Adverse Events; DLT, dose-limiting toxicity; NCI, National Cancer Institute; PT, preferred term; SAE, serious adverse event.

### Example 18: Phase 1/1b Clinical Trial (Part 2) of CD40.9H3×FAP.LP62 as Monotherapy and Combination Therapy in Participants with Advanced Solid Malignancies

Up to three indication-specific expansion cohorts are included for Part 2A monotherapy (CD40.9H3×FAP.LP62) dose expansion and/or for Part 2B combination (with nivolumab) dose expansion (FIG. 15, Part 2) and/or Part 2C combination (with nivolumab and chemotherapy (dose expansion in GC/GEJ (FIG. 16, Part 2). This dose expansion study is designed to obtain additional data on safety and estimate the preliminary efficacy of CD40.9H3×FAP.LP62 doses and schedules.

Each cohort includes up to approximately 40 participants for continued analysis of safety and preliminary analysis of efficacy and biomarkers and to support initial dose finding for further development. Cohorts may enroll either sequentially or in parallel. If multiple cohorts for the same indication are enrolled in parallel, random cohort allocation may be applied with an equal randomization ratio.

All participants complete up to 3 study periods: Screening, Treatment, and Follow-up following end of treatment (EOT) with study intervention, as described in Example 17. The total duration of study participation is approximately 4 years for participants in Part 2 (screening and treatment of up to 2 calendar years and follow-up of up to 2 calendar years).

### Example 19: Characterization of CD40.9H3×FAP.LP62 Structure

The structure of the CD40.9H3×FAP.LP62 bispecific antibody was investigated for different variants, such as modifications to the N-terminus, modifications to the C-terminus, glycosylation, oxidation, deamidation, isomerization, glycation, and free thiols. For purposes of characterization, heavy chain A (HC-A) was defined as the heavy chain of CD40.9H3×FAP.LP62 that didn't contain the FAP scFv, while heavy chain B (HC-B) was defined as the heavy chain of CD40.9H3×FAP.LP62 that did contain the FAP scFv.

### Modifications to the N-Terminus

The light and heavy chains of CD40.9H3×FAP.LP62 contain an N-terminal glutamic acid residue, which was found to be capable of cyclizing to form pyroglutamate (pyroE). Both the cyclized and uncyclized N-terminal variants were detected from the tryptic peptide map. The percentage of each variant was measured by dividing the MS-based peak area of the pyroE peptide by the sum of the peak areas of uncyclized and pyroE peptides in their extracted ion chromatograms. Results are presented in Table 12.

TABLE 12
Relative Levels of N-terminal Pyroglutamate
in the Light and Heavy Chains of
CD40.9H3 × FAP.LP62

| | | Toxicology | Clinical |
|---|---|---|---|
| | N-terminal | batch | batch |
| Chain | peptide | (%) | (%) |
| Light | EIVLTQSPGTLSLSP | 99.1 | 99.2 |
| Chain | GER | | |
| | (SEQ ID NO: 44) | | |
| | pyroEIVLTQSPGTL | 0.9 | 0.8 |
| | SLSPGER | | |
| | (SEQ ID NO: 45) | | |
| | EVQLVESGGGLVQPG | 98.6 | 98.7 |
| | GSLR | | |
| | (SEQ ID NO: 46) | | |
| Heavy | pyroEVQLVESGGGL | 1.4 | 1.3 |
| Chain | VQPGGSLR | | |
| (HC-A | (SEQ ID NO: 47) | | |
| and | | | |
| HC-B) | | | |

The uncyclized glutamic acid was the predominant form at the N-terminus of each chain, with levels consistent between batches, supporting that the toxicology batch was representative of the clinical batch with respect to N-terminal variants.

### Modifications to the C-Terminus

The C-terminus of the CD40.9H3×FAP.LP62 heavy chain A was found to contain two variants: an intact form with glycine, and a variant resulting from glycine clipping and amidation of the preceding proline residue. C-terminal amidation resulted from the reaction catalyzed by peptidylglycine alpha-amidating monooxygenase (PAM) enzyme during cell culture.

Two C-terminal tryptic peptides were detected: SLSLSPG and SLSLSP-amide. The percentage of each variant in the drug substance batches was assessed by dividing the MS-based peak area of the C-terminal amidated peptide by the sum of the peak areas of the 2 peptides in their extracted ion chromatograms. Results in Table 13 indicate that the intact form is the predominant C-terminal form of heavy chain A.

TABLE 13
Relative Levels of C-Terminal Variants
in Heavy Chain A of CD40.9H3 × FAP.LP62

| | C-Terminal | Toxicology Batch | Clinical Batch |
|---|---|---|---|
| Chain | Peptide | (%) | (%) |
| HC-A | SLSLSPG | 93.7 | 84.3 |
| | SLSLSP-amide | 6.3 | 15.7 |

The relative levels of C-terminal amidated form was higher for the clinical batch (15.7%) compared to the toxicology batch (6.3%). This variant is a common post-translational modification in both CHO-derived monoclonal antibodies and endogenous human IgGs, suggesting that the presence of this modification should not pose a safety concern.

No C-terminal variants were detected for HC-B and the light chain.

### Glycosylation

CD40.9H3×FAP.LP62 contains a single N-linked glycosylation site on each heavy chain at Asn304 of the Fc region. The N-linked oligosaccharide profile of CD40.9H3×FAP.LP62 was characterized by HILIC (Hydrophilic Interaction Chromatography) with fluorescent labeling. Briefly, N-glycans were removed from the protein by PNGase F treatment and subsequently labeled with a fluorescent tag. The labeled N-glycans were then separated by HILIC and detected by a fluorescence detector. Individual N-glycan peak areas were integrated and reported as relative percent of the total identified N-glycan peak areas.

The N-glycoforms detected in CD40.9H3×FAP.LP62 were typical of those observed in human monoclonal antibodies. The predominant glycans present were determined to be core-fucosylated bi-antennary GOF and GIF glycans, which accounted for approximately 79% of the total identified glycans for the toxicology batch and about 82% for the clinical batch. Minor glycans identified include afucosylated (G0, G1, G0-GN, Man5), and fucosylated (G0F-GN, G2F, G1F-GN, G0FN) species. Sialylated glycans were not detected in both batches. CD40.9H3×FAP.LP62 was designed with AAS mutations (L234A/L235A/D265S) on the Fc to silence effector functions, and minor differences in N-glycan percentages are not expected to impact biological activity. Overall, the N-glycan profiles for the toxicology batch and the clinical batch were similar.

### Oxidation

CD40.9H3×FAP.LP62 contains 5 methionine (Met) residues in HC-A and 9 Met residues in HC-B that can undergo oxidation to form methionine sulfoxide or methionine sulfone. Met34 of both heavy chains is part of the complementarity-determining region (CDR) of the anti-CD40 Fab domain, while Met505 and Met580 of HC-B are part of the CDR of the anti-FAP scFv. No Met residues are present in the LC. Meanwhile, CD40.9H3×FAP.LP62 also contains tryptophan (Trp) residues (9 Trp in HC-A, 12 Trp in HC-B, and 4 Trp residues in the LC) that can potentially undergo oxidation. Of these residues, only Trp94 and Trp95 of the LC are part of the CDR.

Levels of Met and Trp oxidation were assessed from tryptic peptide mapping with LC-MS analysis by dividing the extracted ion chromatogram peak area of the oxidized peptide by the sum of the peak areas for the oxidized and non-oxidized peptides. The results are summarized in Table 14.

TABLE 14
Levels of Met and Trp Oxidation in CD40.9H3 ×
FAP.LP62 Drug Substance Batches

| | | % Oxidation | |
|---|---|---|---|
| Oxidation Site/ | | Toxicology | Clinical |
| Residue Number | Chain | Batch | Batch |
| Met34 | HC-A/HC-B (CRD) | 0.4 | 0.4 |
| Met83 | HC-A/HC-B | 0.1 | 0.1 |
| Met259 | HC-A/HC-B | 1.4 | 1.3 |
| Met365 | HC-A/HC-B | 0.6 | 0.5 |
| Met435 | HC-A/HC-B | 0.6 | 0.5 |
| Met580 | HC-B (scFv CDR) | 0.4 | 0.5 |
| Trp94 | LC (CDR) | 0.1 | 0.1 |

Oxidation was detected in 6 Met residues. The oxidation levels were low (less than 2% at any individual residue), as typically observed in monoclonal antibodies. For Trp, the total oxidation level was taken as the sum of all observed Trp oxidation products. Oxidation was detected only in Trp94 of the LC at very low levels (0.1%). Similar levels of Met and Trp oxidation are observed between the toxicology batch and the clinical batch, supporting that the toxicology batch was representative of the clinical batch with respect to oxidation levels.

### Deamidation

Deamidation of asparagine (Asn) residues was detected by peptide mapping and LC-MS analysis. Deamidation levels were measured by dividing the peak area of the deamidated peptide by the sum of the peak areas of the unmodified and deamidated peptides in their extracted ion chromatograms. The results are summarized in Table 15.

TABLE 15
Deamidation Levels in CD40.9H3 ×
FAP.LP62 Drug Substance Batches

| Deamidation Site/ | | % Deamidation | |
|---|---|---|---|
| Residue Number | Chain | Toxicology Batch | Clinical Batch |
| Asn93 | LC (CDR) | 0.2 | 0.2 |
| Asn136 | LC | 0.1 | 0.1 |
| Asn77 | HC-A/HC-B | 0.7 | 0.7 |
| Asn84 | HC-A/HC-B | 0.2 | 0.1 |
| Asn293 | HC-A/HC-B | 0.1 | 0.1 |
| Asn322 | HC-A/HC-B | 0.3 | 0.3 |
| Asn332 | HC-A/HC-B | 0.1 | 0.2 |
| Asn368 | HC-A | 0.6 | 0.6 |
| Asn368 | HC-B | 0.5 | 0.5 |
| Asn391, Asn396 | HC-B | 2.6 | 2.4 |
| Asn441 | HC-A/HC-B | 1.1 | 1.1 |
| Asn555 | HC-B | 0.3 | 0.3 |

Overall, low levels (<3%) of deamidation were observed. Deamidation is known to contribute to the acidic charge variants of monoclonal antibodies. In this case, similar deamidation sites and levels were observed between the toxicology and clinical batches, supporting that the toxicology batch is representative of the clinical batch with respect to deamidation levels.

### Isomerization

Aspartic acid (Asp or D) residues can undergo a chemical reaction to form a cyclic imide intermediate (Asu) and isomerize to isoaspartic acid (isoAsp). Most susceptible are sequences containing the DG motif (aspartic acid followed by glycine). In silico modelling did not identify any Asp isomerization hotspots in the CDR regions of CD40.9H3×FAP.LP62. DG motifs are present in the Fc region of the heavy chains (Asp287 and Asp408). Variants resulting from Asp isomerization were detected by peptide mapping and LC-MS analysis on tryptic peptides containing the DG motif. Results summarized in Table 16 show minimal isomerization for CD40.9H3×FAP.LP62, which could also be method-induced. The levels of Asp isomerization and cyclic imide formation were similar between the toxicology and the clinical batches.

TABLE 16
Asp Isomerization and Cyclic Imide
Formation in CD40.9H3 × FAP.LP62

| | | Toxicology Batch | Clinical Batch |
|---|---|---|---|
| Modification | Chain | (%) | (%) |
| Asp287 to isoAsp | HC-A/HC-B | 0.2 | 0.2 |
| Asp287 to Asu | | 1.3 | 0.9 |
| Asp408 to isoAsp | HC-A | ND | ND |
| Asp408 to Asu | | ND | ND |
| Asp408 to isoAsp | HC-B | 0.1 | 0.1 |
| Asp408 to Asu | | 0.4 | 0.4 |

ND: Not detected

### Glycation

Glycation of proteins occurs through a non-enzymatic reaction between the primary amine group of lysine side chains (and/or the N-terminal amino group) and the aldehyde group of reducing sugars such as glucose. Addition of a sugar moiety to the side chain of lysine residues converts the basic amino group into a neutral group, and thus could shift the charge profile of antibodies towards more acidic pI.

CD40.9H3×FAP.LP62 contains 89 lysine residues in total, along with the N-termini of the light and heavy chains, that are possible glycation sites. Although multiple solvent-exposed lysine residues throughout a protein are susceptible to glycation, the levels at any specific residue is typically low. The overall glycation levels in CD40.9H3×FAP.LP62 were estimated based on the intact mass analysis of non-reduced, de-glycosylated samples. Mono-glycated species are detected as a +162 Da peak from the main peak. Glycation levels were determined by taking the ratio of the peak intensity of the glycated peak over the sum of the peak intensities of the main (non-glycated) peak and the glycated peak. The overall glycation level was 13.9% total glycation in the toxicological batch and 13.0% total glycation in the clinical batch. Similar glycation levels were observed between the batches, supporting that the toxicology batch is representative of the clinical batch with respect to glycation levels.

### Free Thiols

All 38 cysteine residues of CD40.9H3×FAP.LP62 were expected to be involved in disulfide bonds. Trace levels of unpaired cysteines were measured by fluorescent labeling of free thiols using N-(iodoacetyl)-N′-(5-sulfo-1-naphthyl)ethylenediamine (IA-EDANS), under mild denaturing conditions, followed by analysis using reversed phase chromatography with a fluorescence detector. The percentage of free thiol was calculated by comparing the IA-EDANS labeled sample (non-reduced) with a control sample that was fully reduced with dithiothreitol (DTT) prior to labeling with IA-EDANS. The fluorescence peak area ratio between the non-reduced and fully reduced sample gave the percentage of free thiol. Analysis of the toxicology batch and clinical batch showed comparable levels of free thiols (Table 17).

TABLE 17
Free Thiols in CD40.9H3 × FAP.LP62 Drug Substance Batches

| Drug Substance Batch | Thiol/IgG | % Thiol |
|---|---|---|
| Toxicology Batch | 0.48 | 1.26 |
| Clinical Batch | 0.49 | 1.29 |

X. TABLE OF CERTAIN SEQUENCES
The following table provides a listing of certain sequences
referenced herein.

| SEQ | | |
|---|---|---|
| ID | | |
| NO: | Description | Sequence |
| 1 | anti-CD40 9H3 HCDR1, Kabat | GYSMN |
| 2 | anti-CD40 9H3 HCDR2 N52S, | YISSYESTIYYADSVKG |
| | Kabat | |
| 3 | anti-CD40 9H3 HCDR3, Kabat | SRITLVRGVPRYFDL |
| 4 | anti-CD40 9H3 LCDR1, Kabat | RASQSVSSYLA |
| 5 | anti-CD40 9H3 LCDR2, Kabat | DASNRAT |
| 6 | anti-CD40 9H3 LCDR3, Kabat | QQRSNWWT |
| 7 | anti-CD40 9H3 HCDR1, IMGT | GFTFIGYS |
| 8 | anti-CD40 9H3 HCDR2 N52S, | ISSYESTI |
| | IMGT | |
| 9 | anti-CD40 9H3 HCDR3, IMGT | ARSRITLVRGVPRYFDL |
| 10 | anti-CD40 9H3 LCDR1, IMGT | QSVSSY |
| 11 | anti-CD40 9H3 LCDR2, IMGT | DAS |
| 12 | anti-CD40 9H3 LCDR3, IMGT | QQRSNWWT |
| 13 | anti-CD40 9H3 N52S heavy | EVQLVESGGGLVQPGGSLRLSCAASGFTFIGYSMNWVRQAPGK |
| | chain variable region | GLEWVSYISSYESTIYYADSVKGRFTISRDNAKNSLYLQMNSL |
| | | RAEDTAVYYCARSRITLVRGVPRYFDLWGRGTLVTVSS |
| 14 | anti-CD40 9H3 light chain | EIVLTQSPGTLSLSPGERATLSCRASQSVSSYLAWYQQKPGQA |
| | variable region | PRLLIYDASNRATGIPARFSGSGSGTDFTLTISSLQPEDFAVY |
| | | YCQQRSNWWTFGQGTKVEIK |
| 15 | anti-CD40 9H3 N52S IgG1 | EVQLVESGGGLVQPGGSLRLSCAASGFTFIGYSMNWVRQAPGK |
| | L234A, L235A, D265S, T350V, | GLEWVSYISSYESTIYYADSVKGRFTISRDNAKNSLYLQMNSL |
| | T366L, K392L, and T394W | RAEDTAVYYCARSRITLVRGVPRYFDLWGRGTLVTVSSASTKG |
| | (“CD40.9H3 × FAP.LP62” heavy | PSVFPLAPSSKSTSGGTAALGCLVKDYFPEPVTVSWNSGALTS |
| | chain 1 (HC1)) | GVHTFPAVLQSSGLYSLSSVVTVPSSSLGTQTYICNVNHKPSN |
| | | TKVDKKVEPKSCDKTHTCPPCPAPEAAGGPSVFLFPPKPKDTL |
| | | MISRTPEVTCVVVSVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYVLPPSREEMTKNQVSLLCLVKGFYPSDIAVE |
| | | WESNGQPENNYLTWPPVLDSDGSFFLYSKLTVDKSRWQQGNVF |
| | | SCSVMHEALHNHYTQKSLSLSPG |
| 16 | anti-CD40 9H3 kappa light | EIVLTQSPGTLSLSPGERATLSCRASQSVSSYLAWYQQKPGQA |
| | chain (“CD40.9H3 × FAP.LP62” | PRLLIYDASNRATGIPARFSGSGSGTDFTLTISSLQPEDFAVY |
| | light chain (LC)) | YCQQRSNWWTFGQGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| 17 | anti-FAP LP62 HCDR1, Kabat | SRGMS |
| 18 | anti-FAP LP62 HCDR2, Kabat | FISSSGTYVYYADSVKG |
| 19 | anti-FAP LP62 HCDR3, Kabat | DLPRTGVYFGMDV |
| 20 | anti-FAP LP62 LCDR1, Kabat | RASQSIFNYLN |
| 21 | anti-FAP LP62 LCDR2, Kabat | GASTLQS |
| 22 | anti-FAP LP62 LCDR3, Kabat | QESYSTPYT |
| 23 | anti-FAP LP62 HCDR1, IMGT | GFTFSSRG |
| 24 | anti-FAP LP62 HCDR2, IMGT | ISSSGTYV |
| 25 | anti-FAP LP62 HCDR3, IMGT | ARDLPRTGVYFGMDV |
| 26 | anti-FAP LP62 LCDR1, IMGT | QSIFNY |
| 27 | anti-FAP LP62 LCDR2, IMGT | GAS |
| 28 | anti-FAP LP62 LCDR3, IMGT | QESYSTPYT |
| 29 | anti-FAP LP62 heavy chain | EVQLLESGGGLVQPGGSLRLSCAASGFTFSSRGMSWVRQAPGK |
| | variable region | GLEWVSFISSSGTYVYYADSVKGRFTISRDNSKNTLYLQMNSL |
| | | RAEDTAVYYCARDLPRTGVYFGMDVWGQGTTVTVSS |
| 30 | anti-FAP LP62 light chain | DIQMTQSPSSLSASVGDRVTITCRASQSIFNYLNWYQQKPGKA |
| | variable region | PKLLIYGASTLQSGVPSRFSGSGSGTDFTLTISSLQPEDFATY |
| | | YCQESYSTPYTFGQGTKVEIK |
| 31 | anti-FAP LP62 heavy chain | EVQLLESGGGLVQPGGSLRLSCAASGFTFSSRGMSWVRQAPGK |
| | variable region, G44C | CLEWVSFISSSGTYVYYADSVKGRFTISRDNSKNTLYLQMNSL |
| | | RAEDTAVYYCARDLPRTGVYFGMDVWGQGTTVTVSS |
| 32 | anti-FAP LP62 light chain | DIQMTQSPSSLSASVGDRVTITCRASQSIFNYLNWYQQKPGKA |
| | variable region, Q100C | PKLLIYGASTLQSGVPSRFSGSGSGTDFTLTISSLQPEDFATY |
| | | YCQESYSTPYTFGCGTKVEIK |
| 33 | anti-FAP LP62 scFv (VH- | EVQLLESGGGLVQPGGSLRLSCAASGFTFSSRGMSWVRQAPGK |
| | G44C, VL-Q100C) | CLEWVSFISSSGTYVYYADSVKGRFTISRDNSKNTLYLQMNSL |
| | | RAEDTAVYYCARDLPRTGVYFGMDVWGQGTTVTVSSGGGGSGG |
| | | GGSGGGGSGGGGSDIQMTQSPSSLSASVGDRVTITCRASQSIF |
| | | NYLNWYQQKPGKAPKLLIYGASTLQSGVPSRFSGSGSGTDFTL |
| | | TISSLQPEDFATYYCQESYSTPYTFGCGTKVEIK |
| 34 | anti-CD40 9H3 N52S IgG1 | EVQLVESGGGLVQPGGSLRLSCAASGFTFIGYSMNWVRQAPGK |
| | L234A, L235A, D265S, T350V, | GLEWVSYISSYESTIYYADSVKGRFTISRDNAKNSLYLQMNSL |
| | L351Y, F405A, and Y407V/ | RAEDTAVYYCARSRITLVRGVPRYFDLWGRGTLVTVSSASTKG |
| | FAP LP62 scFv (VH-G44C, | PSVFPLAPSSKSTSGGTAALGCLVKDYFPEPVTVSWNSGALTS |
| | VL-Q100C) | GVHTFPAVLQSSGLYSLSSVVTVPSSSLGTQTYICNVNHKPSN |
| | (“CD40.9H3 × FAP.LP62” | TKVDKKVEPKSCDKTHTCPPCPAPEAAGGPSVFLFPPKPKDTL |
| | heavy chain 2 (HC2)-scFv) | MISRTPEVTCVVVSVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYVYPPSREEMTKNQVSLTCLVKGFYPSDIAVE |
| | | WESNGQPENNYKTTPPVLDSDGSFALVSKLTVDKSRWQQGNVF |
| | | SCSVMHEALHNHYTQKSLSLSPGGGSGGGGSGGGGSGGGGSEV |
| | | QLLESGGGLVQPGGSLRLSCAASGFTFSSRGMSWVRQAPGKCL |
| | | EWVSFISSSGTYVYYADSVKGRFTISRDNSKNTLYLQMNSLRA |
| | | EDTAVYYCARDLPRTGVYFGMDVWGQGTTVTVSSGGGGSGGGG |
| | | SGGGGSGGGGSDIQMTQSPSSLSASVGDRVTITCRASQSIFNY |
| | | LNWYQQKPGKAPKLLIYGASTLQSGVPSRFSGSGSGTDFTLTI |
| | | SSLQPEDFATYYCQESYSTPYTFGCGTKVEIK |
| 35 | Linker (between Fc and | GGSGGGGSGGGGSGGGGS |
| | scFv) | |
| 36 | Mature human CD40 | EPPTACREKQYLINSQCCSLCQPGQKLVSDCTEFTETECLPCG |
| | | ESEFLDTWNRETHCHQHKYCDPNLGLRVQQKGTSETDTICTCE |
| | | EGWHCTSEACESCVLHRSCSPGFGVKQIATGVSDTICEPCPVG |
| | | FFSNVSSAFEKCHPWTSCETKDLVVQQAGTNKTDVVCGPQDRL |
| | | RALVVIPIIFGILFAILLVLVFIKKVAKKPTNKAPHPKQEPQE |
| | | INFPDDLPGSNTAAPVQETLHGCQPVTQEDGKESRISVQERQ |
| 37 | Human FAP | MKTWVKIVFGVATSAVLALLVMCIVLRPSRVHNSEENTMRALT |
| | | LKDILNGTFSYKTFFPNWISGQEYLHQSADNNIVLYNIETGQS |
| | | YTILSNRTMKSVNASNYGLSPDRQFVYLESDYSKLWRYSYTAT |
| | | YYIYDLSNGEFVRGNELPRPIQYLCWSPVGSKLAYVYQNNIYL |
| | | KQRPGDPPFQITFNGRENKIFNGIPDWVYEEEMLATKYALWWS |
| | | PNGKFLAYAEFNDTDIPVIAYSYYGDEQYPRTINIPYPKAGAK |
| | | NPVVRIFIIDTTYPAYVGPQEVPVPAMIASSDYYFSWLTWVTD |
| | | ERVCLQWLKRVQNVSVLSICDFREDWQTWDCPKTQEHIEESRT |
| | | GWAGGFFVSTPVFSYDAISYYKIFSDKDGYKHIHYIKDTVENA |
| | | IQITSGKWEAINIFRVTQDSLFYSSNEFEEYPGRRNIYRISIG |
| | | SYPPSKKCVTCHLRKERCQYYTASFSDYAKYYALVCYGPGIPI |
| | | STLHDGRTDQEIKILEENKELENALKNIQLPKEEIKKLEVDEI |
| | | TLWYKMILPPQFDRSKKYPLLIQVYGGPCSQSVRSVFAVNWIS |
| | | YLASKEGMVIALVDGRGTAFQGDKLLYAVYRKLGVYEVEDQIT |
| | | AVRKFIEMGFIDEKRIAIWGWSYGGYVSSLALASGTGLFKCGI |
| | | AVAPVSSWEYYASVYTERFMGLPTKDDNLEHYKNSTVMARAEY |
| | | FRNVDYLLIHGTADDNVHFQNSAQIAKALVNAQVDFQAMWYSD |
| | | QNHGLSGLSTNHLYTHMTHFLKQCFSLSD |
| 38 | Analog of MP0317 (see, | SGGSDLGKKLLEAARAGQDDEVRELLKAGADVNAKDYFSHTPL |
| | e.g., Rigamonti et al., | HLAARNGHLKIVEVLLKAGADVNAKDFAGKTPLHLAAADGHLE |
| | Cancer Immunol. Res., | IVEVLLKAGADVNAQDIFGKTPADIAADAGHEDIAEVLQKAAG |
| | 10:626-40 (2022)) | SPTPTPTTPTPTPTTPTPTPTGSDLGKKLLEAARAGQDDEVRI |
| | | LLAAGADVNAKDVLGWTPLHLAAFEGHLEIVEVLLKAGADVNA |
| | | KDKKGWTPLQLAARTGHLEIVEVLLKAGADVNAKDHIGATPLH |
| | | LAAWQGHPEIVEVLLKAGADVNAQDKSGKTPADLAADAGHEDI |
| | | AEVLQKAAGSPTPTPTTPTPTPTTPTPTPTGSDLGKKLLQAAR |
| | | AGQLDEVRELLKAGADVNAKDTWGFTPLHIAAESGHLEIVEVL |
| | | LKAGADVNAKDVQGRTPLHIAAHSGHLEIVEVLLKAGADVNAK |
| | | DFRGWTPLHLAAWSGHLEIVEILLKAGADVNAQDKSGKTPADL |
| | | AARAGHQDIAEVLQKAAGSPTPTPTTPTPTPTTPTPTPTGSDL |
| | | GKKLLQAARAGQLDEVRELLKAGADVNAKDTWGFTPLHIAAES |
| | | GHLEIVEVLLKAGADVNAKDVQGRTPLHIAAHSGHLEIVEVLL |
| | | KAGADVNAKDFRGWTPLHLAAWSGHLEIVEILLKAGADVNAQD |
| | | KSGKTPADLAARAGHQDIAEVLQKAA |
| 39 | analog of RO7300490, FAP | QVQLVQSGAEVKKPGASVKVSCKASGYTLTDYNMDWVRQAPGQ |
| | VH-kappa (see, e.g., WO | GLEWIGDIYPNTGGTIYNQKFKGRVTMTIDTSTSTVYMELSSL |
| | 2018/185045, WO | RSEDTAVYYCTRFRGIHYAMDYWGQGTTVTVSSASVAAPSVFI |
| | 2022/101458) | FPPSDEQLKSGTASVVCLLNNFYPREAKVQWKVDNALQSGNSQ |
| | | ESVTEQDSKDSTYSLSSTLTLSKADYEKHKVYACEVTHQGLSS |
| | | PVTKSFNRGEC |
| 40 | analog of RO7300490, CD40 | QVQLVQSGAEVKKPGASVKVSCKASGYSFTGYYIHWVRQ |
| | HC-FAP VL-CHI (see, e.g., | APGQSLEWMGRVIPNAGGTSYNQKFKGRVTLTVDKSIST |
| | WO 2018/185045, WO | AYMELSRLRSDDTAVYYCAREGIYWWGQGTTVTVSSAST |
| | 2022/101458) | KGPSVFPLAPSSKSTSGGTAALGCLVEDYFPEPVTVSWN |
| | | SGALTSGVHTFPAVLQSSGLYSLSSVVTVPSSSLGTQTY |
| | | ICNVNHKPSNTKVDEKVEPKSCDKTHTCPPCPAPEAAGG |
| | | PSVFLFPPKPKDTLMISRTPEVTCVVVDVSHEDPEVKFN |
| | | WYVDGVEVHNAKTKPREEQYNSTYRVVSVLTVLHQDWLN |
| | | GKEYKCKVSNKALGAPIEKTISKAKGQPREPQVYTLPPC |
| | | RDELTKNQVSLWCLVKGFYPSDIAVEWESNGQPENNYKT |
| | | TPPVLDSDGSFFLYSKLTVDKSRWQQGNVFSCSVMHEAL |
| | | HNHYTQKSLSLSPGGGGGSGGGGSGGGGSGGGGSEIVLT |
| | | QSPATLSLSPGERATLSCRASESVDNYGLSFINWFQQKP |
| | | GQAPRLLIYGTSNRGSGIPARFSGSGSGTDFTLTISSLE |
| | | PEDFAVYFCQQSNEVPYTFGGGTKVEIKSSASTKGPSVF |
| | | PLAPSSKSTSGGTAALGCLVKDYFPEPVTVSWNSGALTS |
| | | GVHTFPAVLQSSGLYSLSSVVTVPSSSLGTQTYICNVNH |
| | | KPSNTKVDKKVEPKSC |
| 41 | analog of RO7300490, CD40 | QVQLVQSGAEVKKPGASVKVSCKASGYSFTGYYIHWVRQ |
| | HC (see, e.g., WO | APGQSLEWMGRVIPNAGGTSYNQKFKGRVTLTVDKSIST |
| | 2018/185045, WO | AYMELSRLRSDDTAVYYCAREGIYWWGQGTTVTVSSAST |
| | 2022/101458) | KGPSVFPLAPSSKSTSGGTAALGCLVEDYFPEPVTVSWN |
| | | SGALTSGVHTFPAVLQSSGLYSLSSVVTVPSSSLGTQTY |
| | | ICNVNHKPSNTKVDEKVEPKSCDKTHTCPPCPAPEAAGG |
| | | PSVFLFPPKPKDTLMISRTPEVTCVVVDVSHEDPEVKFN |
| | | WYVDGVEVHNAKTKPREEQYNSTYRVVSVLTVLHQDWLN |
| | | GKEYKCKVSNKALGAPIEKTISKAKGQPREPQVCTLPPS |
| | | RDELTKNQVSLSCAVKGFYPSDIAVEWESNGQPENNYKT |
| | | TPPVLDSDGSFFLVSKLTVDKSRWQQGNVFSCSVMHEAL |
| | | HNHYTQKSLSLSPG |
| 42 | analog of RO7300490, CD40 | DIVMTQTPLSLSVTPGQPASISCRSSQSLVHSNGNTFLH |
| | LC (see, e.g., WO | WYLQKPGQSPQLLIYTVSNRFSGVPDRFSGSGSGTDFTL |
| | 2018/185045, WO | KISRVEAEDVGVYFCSQTTHVPWTFGGGTKVEIKRTVAA |
| | 2022/101458) | PSVFIFPPSDRKLKSGTASVVCLLNNFYPREAKVQWKVD |
| | | NALQSGNSQESVTEQDSKDSTYSLSSTLTLSKADYEKHK |
| | | VYACEVTHQGLSSPVTKSFNRGEC |
| 43 | anti-CD40 9H3 N52S IgG1 | EVQLVESGGGLVQPGGSLRLSCAASGFTFIGYSMNWVRQAPGK |
| | L234A, L235A, D265S, T350V, | GLEWVSYISSYESTIYYADSVKGRFTISRDNAKNSLYLQMNSL |
| | T366L, K392L, and T394W | RAEDTAVYYCARSRITLVRGVPRYFDLWGRGTLVTVSSASTKG |
| | (“CD40.9H3 × FAP.LP62” | PSVFPLAPSSKSTSGGTAALGCLVKDYFPEPVTVSWNSGALTS |
| | heavy chain 1 (HC1)), | GVHTFPAVLQSSGLYSLSSVVTVPSSSLGTQTYICNVNHKPSN |
| | glycine-clipped | TKVDKKVEPKSCDKTHTCPPCPAPEAAGGPSVFLFPPKPKDTL |
| | | MISRTPEVTCVVVSVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYVLPPSREEMTKNQVSLLCLVKGFYPSDIAVE |
| | | WESNGQPENNYLTWPPVLDSDGSFFLYSKLTVDKSRWQQGNVF |
| | | SCSVMHEALHNHYTQKSLSLSP |
| 44 | Light Chain N-terminus | EIVLTQSPGTLSLSPGER |
| 45 | Light Chain N-terminus | XIVLTQSPGTLSLSPGER, wherein X is pyroE |
| | variant | |
| 46 | Heavy Chain N-terminus | EVQLVESGGGLVQPGGSLR |
| 47 | Heavy Chain N-terminus | XVQLVESGGGLVQPGGSLR, wherein X is pyroE |
| | variant | |
| 48 | HC1 C-terminus | SLSLSPG |
| 49 | HC1 C-terminus variant | SLSLSP |
| 50 | CH1 region | ASTKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKKV |
| 51 | Heavy Chain 1 (HC1) Fc | EPKSCDKTHTCPPCPAPEAAGGPSVFLFPPKPKDTLMISRTPE |
| | | VTCVVVSVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYVLPPSREEMTKNQVSLLCLVKGFYPSDIAVEWESNGQP |
| | | ENNYLTWPPVLDSDGSFFLYSKLTVDKSRWQQGNVFSCSVMHE |
| | | ALHNHYTQKSLSLSPG |
| 52 | Heavy Chain 2 (HC2) Fc | EPKSCDKTHTCPPCPAPEAAGGPSVFLFPPKPKDTLMISRTPE |
| | | VTCVVVSVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYVYPPSREEMTKNQVSLTCLVKGFYPSDIAVEWESNGQP |
| | | ENNYKTTPPVLDSDGSFALVSKLTVDKSRWQQGNVFSCSVMHE |
| | | ALHNHYTQKSLSLSPG |
| 53 | CD40-binding Fab heavy | EVQLVESGGGLVQPGGSLRLSCAASGFTFIGYSMNWVRQAPGK |
| | chain | GLEWVSYISSYESTIYYADSVKGRFTISRDNAKNSLYLQMNSL |
| | | RAEDTAVYYCARSRITLVRGVPRYFDLWGRGTLVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKV |
| 54 | Peptide linker | GGGGSGGGGSGGGGSGGGGS |
| 55 | Heavy Chain 1 (HC1) Fc, | EPKSCDKTHTCPPCPAPEAAGGPSVFLFPPKPKDTLMISRTPE |
| | glycine-clipped | VTCVVVSVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYVLPPSREEMTKNQVSLLCLVKGFYPSDIAVEWESNGQP |
| | | ENNYLTWPPVLDSDGSFFLYSKLTVDKSRWQQGNVFSCSVMHE |
| | | ALHNHYTQKSLSLSP |
| 56 | Heavy Chain 2 (HC2) Fc, | EPKSCDKTHTCPPCPAPEAAGGPSVFLFPPKPKDTLMISRTPE |
| | glycine-clipped | VTCVVVSVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYVYPPSREEMTKNQVSLTCLVKGFYPSDIAVEWESNGQP |
| | | ENNYKTTPPVLDSDGSFALVSKLTVDKSRWQQGNVFSCSVMHE |
| | | ALHNHYTQKSLSLSP |
| 57 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVYA |
| | | CEVTHQGLSSPVTKSFNRGEC |
| 58 | anti-CD40 9H3 N52S IgG1 | EVQLVESGGGLVQPGGSLRLSCAASGFTFIGYSMNWVRQAPGK |
| | L234A, L235A, D265S, T350V, | GLEWVSYISSYESTIYYADSVKGRFTISRDNAKNSLYLQMNSL |
| | L351Y, F405A, and Y407V/ | RAEDTAVYYCARSRITLVRGVPRYFDLWGRGTLVTVSSASTKG |
| | FAP LP62 scFv (VH-G44C, | PSVFPLAPSSKSTSGGTAALGCLVKDYFPEPVTVSWNSGALTS |
| | VL-Q100C), glycine-clipped | GVHTFPAVLQSSGLYSLSSVVTVPSSSLGTQTYICNVNHKPSN |
| | (“CD40.9H3 × FAP.LP62” | TKVDKKVEPKSCDKTHTCPPCPAPEAAGGPSVFLFPPKPKDTL |
| | heavy chain 2 (HC2)-scFv), | MISRTPEVTCVVVSVSHEDPEVKFNWYVDGVEVHNAKTKPREE |
| | glycine-clipped | QYNSTYRVVSVLTVLHQDWLNGKEYKCKVSNKALPAPIEKTIS |
| | | KAKGQPREPQVYVYPPSREEMTKNQVSLTCLVKGFYPSDIAVE |
| | | WESNGQPENNYKTTPPVLDSDGSFALVSKLTVDKSRWQQGNVF |
| | | SCSVMHEALHNHYTQKSLSLSPGGSGGGGSGGGGSGGGGSEVQ |
| | | LLESGGGLVQPGGSLRLSCAASGFTFSSRGMSWVRQAPGKCLE |
| | | WVSFISSSGTYVYYADSVKGRFTISRDNSKNTLYLQMNSLRAE |
| | | DTAVYYCARDLPRTGVYFGMDVWGQGTTVTVSSGGGGSGGGGS |
| | | GGGGSGGGGSDIQMTQSPSSLSASVGDRVTITCRASQSIFNYL |
| | | NWYQQKPGKAPKLLIYGASTLQSGVPSRFSGSGSGTDFTLTIS |
| | | SLQPEDFATYYCQESYSTPYTFGCGTKVEIK |

We claim:

1. A multispecific antibody comprising a first antigen binding domain that binds CD40, a second antigen binding domain that binds CD40, and a third antigen binding domain that binds fibroblast activation protein alpha (FAP), wherein the third antigen binding domain is a single chain variable region (scFv).

2. The multispecific antibody of claim 1, wherein the first antigen binding domain comprises a first heavy chain variable region and a first light chain variable region, the second antigen binding domain comprises a second heavy chain variable region and a second light chain variable region, and the scFv comprises a third heavy chain variable region and a third light chain variable region.

3. The multispecific antibody of claim 2, wherein the multispecific antibody comprises:
a) a first heavy chain comprising the first heavy chain variable region fused to a first heavy chain constant region;
b) a first light chain comprising the first light chain variable region fused to a first light chain constant region;
c) a second heavy chain comprising the second heavy chain variable region fused to a second heavy chain constant region; and
d) a second light chain comprising the second light chain variable region fused to a second light chain constant region;
wherein the first heavy chain constant region and the second heavy chain constant region are the same or different.

4. The multispecific antibody of claim 3, wherein the scFv is fused to the C-terminus of the first heavy chain constant region.

5. The multispecific antibody of claim 3 or claim 4, wherein the first heavy chain constant region and the second heavy chain constant region are different.

6. The multispecific antibody of claim 3 or claim 4, wherein each of the first heavy chain constant region and the second heavy chain constant region comprises a heavy chain constant region 1 (CH1) and a Fc polypeptide chain, and
wherein the first binding domain and second binding domain each comprise a Fab, wherein each Fab comprises:
(i) a Fab heavy chain comprising the first binding domain or second variable heavy chain and the CH1, and
(ii) the first or second light chain,
wherein each Fab is the same.

7. The multispecific antibody of claim 6, wherein each CH1 is an IgG1 CH1.

8. The multispecific antibody of claim 6 or claim 7, wherein each CH1 comprises the amino acid sequence set forth in SEQ ID NO: 50.

9. The multispecific antibody of any one of claims 6-8, wherein the Fc polypeptide chain of the first and second heavy chain constant region are different.

10. The multispecific antibody of any one of claims 3-9, wherein the first heavy chain constant region and the second heavy chain constant region form a heterodimer.

11. The multispecific antibody of claim 5 or claim 6, wherein the Fc polypeptide chain of the first heavy chain constant region comprises at least one first heterodimerization mutation and the Fc polypeptide chain of the second heavy chain constant region comprises at least one second heterodimerization mutation.

12. The multispecific antibody of any one of claims 3-11, wherein the first heavy chain constant region comprises at least one first heterodimerization mutation and the second heavy chain constant region comprises at least one second heterodimerization mutation.

13. The multispecific antibody of claim 11 or claim 12, wherein the at least one first heterodimerization mutation and at least one second heterodimerization mutation are with reference to an Fc polypeptide chain of an IgG1 constant region.

14. The multispecific antibody of any one of claims 11-13, wherein at least one first heterodimerization mutation comprises T366W and at least one second heterodimerization mutation comprises one or more of T366S, L368A, and/or Y407V, or wherein at least one first heterodimerization mutation comprises one or more of T366S, L368A, and/or Y407V and at least one second heterodimerization mutation comprises T366W, wherein mutation positions are according to Kabat.

15. The multispecific antibody of any one of claims 11-13, wherein at least one first heterodimerization mutation comprises T366W and at least one second heterodimerization mutation comprises T366S, L368A, and Y407V, or wherein at least one first heterodimerization mutation comprises T366S, L368A, and Y407V and at least one second heterodimerization mutation comprises T366W, wherein mutation positions are according to Kabat.

16. The multispecific antibody of any one of claims 11-13, wherein at least one first heterodimerization mutation comprises one or more of T350V, L351Y, S400E, F405A, and/or Y407V and at least one second heterodimerization mutation comprises one or more of T350V, T366L, N390R, K392M, K392L, and/or T394W, or wherein at least one first heterodimerization mutation comprises one or more of T350V, T366L, N390R, K392M, K392L, and/or T394W and at least one second heterodimerization mutation comprises one or more of T350V, L351Y, S400E, F405A, and/or Y407V, wherein mutation positions are according to Kabat.

17. The multispecific antibody of any one of claims 11-13, wherein at least one first heterodimerization mutation comprises T350V, L351Y, F405A, and Y407V and at least one second heterodimerization mutation comprises T350V, T366L, K392L, and T394W, or wherein at least one first heterodimerization mutation comprises one or more of T350V, T366L, K392L, and T394W and at least one second heterodimerization mutation comprises one or more of T350V, L351Y, F405A, and Y407V, wherein mutation positions are according to Kabat.

18. The multispecific antibody of any one of claims 3-17, wherein the first heavy chain constant region and the second heavy chain constant region are IgG1 constant regions.

19. The multispecific antibody of any one of claims 3-18, wherein the first heavy chain constant region and the second heavy chain constant region do not bind FcγR or have reduced binding to FcγR compared to wild-type constant regions of the same isotype.

20. The multispecific antibody of any one of claims 6-19, wherein each Fc polypeptide chain is effectorless.

21. The multispecific antibody of any one of claims 6-20, wherein each Fc polypeptide chain comprises L234A, L235A, and/or D265S substitutions, wherein substitution positions are according to Kabat.

22. The multispecific antibody of any one of claims 6-21, wherein the Fc polypeptide chain of the first heavy chain constant region comprises an amino acid sequence at least 85%, at least 90%, or at least 95% identical to the amino acid sequence of SEQ ID NO: 51 or SEQ ID NO: 52, and wherein the Fc polypeptide chain of the second heavy chain constant region comprises an amino acid sequence at least 85%, at least 90%, or at least 95% identical to the amino acid sequence of the other of SEQ ID NO: 51 or SEQ ID NO: 52.

23. The multispecific antibody of any one of claims 6-22, wherein the Fc polypeptide chain of the first heavy chain constant region comprises the amino acid sequence of SEQ ID NO: 51 or SEQ ID NO: 52, and wherein the Fc polypeptide chain of the second heavy chain constant region comprises the amino acid sequence of the other of SEQ ID NO: 51 or SEQ ID NO: 52.

24. The multispecific antibody of any one of claims 6-22, wherein the Fc polypeptide chain of the first heavy chain constant region comprises the amino acid sequence of SEQ ID NO: 55 or SEQ ID NO: 56, and wherein the Fc polypeptide chain of the second heavy chain constant region comprises the amino acid sequence of the other of SEQ ID NO: 55 or SEQ ID NO: 56.

25. The multispecific antibody of any one of claims 1-24, wherein the first antigen binding domain comprises a first heavy chain variable region (VH) and a first light chain variable region (VL), wherein:
a) the first heavy chain variable region comprises a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and
b) the first light chain variable region comprises a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

26. The multispecific antibody of claim 25, wherein the first heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the first light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

27. The multispecific antibody of claim 25, wherein the first heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the first light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

28. The multispecific antibody of any one of claims 25-27, wherein the first heavy chain variable region comprises an amino acid sequence at least 85%, at least 90%, or at least 95% identical to the amino acid sequence of SEQ ID NO: 13; and the first light chain variable region comprises an amino acid sequence at least 85%, at least 90%, or at least 95% identical to the amino acid sequence of SEQ ID NO: 14.

29. The multispecific antibody of any one of claims 1-28, wherein the first antigen binding domain comprises a first heavy chain variable region comprising the amino acid sequence of SEQ ID NO: 13, and a first light chain variable region comprising the amino acid sequence of SEQ ID NO: 14.

30. The multispecific antibody of any one of claims 1-29, wherein the second antigen binding domain comprises a second heavy chain variable region and a second light chain variable region, wherein:
a) the second heavy chain variable region comprises a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and
b) the second light chain variable region comprises a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

31. The multispecific antibody of claim 30, wherein the second heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the second light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

32. The multispecific antibody of claim 30, wherein the second heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the second light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

33. The multispecific antibody of any one of claims 30-32, wherein the second heavy chain variable region comprises an amino acid sequence at least 85%, at least 90%, or at least 95% identical to the amino acid sequence of SEQ ID NO: 13; and the second light chain variable region comprises an amino acid sequence at least 85%, at least 90%, or at least 95% identical to the amino acid sequence of SEQ ID NO: 14.

34. The multispecific antibody of any one of claims 1-33, wherein the second antigen binding domain comprises a second heavy chain variable region comprising the amino acid sequence of SEQ ID NO: 13, and a second light chain variable region comprising the amino acid sequence of SEQ ID NO: 14.

35. The multispecific antibody of any one of claims 6-34, wherein each Fab heavy chain comprises an amino acid sequence at least 85%, at least 90%, or at least 95% identical to the amino acid sequence of SEQ ID NO: 53.

36. The multispecific antibody of any one of claims 6-35, wherein each Fab heavy chain comprises the amino acid sequence of SEQ ID NO: 53.

37. The multispecific antibody of any one of claims 1-36, wherein the scFv comprises a third heavy chain variable region and a third light chain variable region, wherein:
a) the third heavy chain variable region comprises a heavy chain complementarity determining region (HCDR) 1 comprising the amino acid sequence of SEQ ID NO: 17 or 23, a HCDR2 comprising the amino acid sequence of SEQ ID NO: 18 or 24, and a HCDR3 comprising the amino acid sequence of SEQ ID NO: 19 or 25; and
b) the third light chain variable region comprises a LCDR1 comprising the amino acid sequence of SEQ ID NO: 20 or 26, a LCDR2 comprising the amino acid sequence of SEQ ID NO: 21 or 27, and a LCDR3 comprising the amino acid sequence of SEQ ID NO: 22 or 28.

38. The multispecific antibody of claim 37, wherein the third heavy chain variable region comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19; and the third light chain variable region comprises a LCDR1 comprising the amino acid sequence of SEQ ID NO: 20, a LCDR2 comprising the amino acid sequence of SEQ ID NO: 21, and a LCDR3 comprising the amino acid sequence of SEQ ID NO: 22.

39. The multispecific antibody of claim 37, wherein the third heavy chain variable region comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25; and the third light chain variable region comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

40. The multispecific antibody of any one of claims 37-39, wherein the third heavy chain variable region comprises an amino acid sequence at least 85%, at least 90%, or at least 95% identical to the amino acid sequence of SEQ ID NO: 29 or 31; and the third light chain variable region comprises an amino acid sequence at least 85%, at least 90%, or at least 95% identical to the amino acid sequence of SEQ ID NO: 30 or 32.

41. The multispecific antibody of any one of claims 1-40, wherein the third antigen binding domain comprises a third heavy chain variable region comprising the amino acid sequence of SEQ ID NO: 29 or 31; and a third light chain variable region comprising the amino acid sequence of SEQ ID NO: 30 or 32.

42. The multispecific antibody of any one of claims 1-41, wherein the third antigen binding domain comprises a third heavy chain variable region comprising the amino acid sequence of SEQ ID NO: 29; and a third light chain variable region comprising the amino acid sequence of SEQ ID NO: 30.

43. The multispecific antibody of any one of claims 1-41, wherein the third antigen binding domain comprises a third heavy chain variable region comprises the amino acid sequence of SEQ ID NO: 31; and a third light chain variable region comprising the amino acid sequence of SEQ ID NO: 32.

44. The multispecific antibody of any one of claims 1-43, wherein the third antigen binding domain is an scFv comprising the amino acid sequence of SEQ ID NO: 33.

45. The multispecific antibody of any one of claims 6-44, wherein the multispecific antibody comprises:
a) the first and second antigen binding domains that binds CD40, and each comprises the Fab; and
b) a heterodimeric Fc region comprising the Fc polypeptide chain of the first heavy chain constant region and the Fc polypeptide chain of the second heavy chain constant region, where the Fc polypeptide chain of the first heavy chain constant region and Fc polypeptide chain of the second heavy chain constant region are each linked to the C-terminus of one of the Fab heavy chains and are linked to different Fab heavy chains; and
b) the scFv of the third binding domain that is fused to the C-terminus of the Fc polypeptide chain of the first heavy chain constant region.

46. The multispecific antibody of any one of claims 6-45, wherein the Fab comprises the Fab heavy chain comprising the amino acid sequence of SEQ ID NO: 53 and the first light chain comprising the amino acid sequence of SEQ ID NO: 16.

47. The multispecific antibody of any one of claims 6-46, wherein the multispecific antibody comprises a first polypeptide, a second polypeptide, and a third polypeptide, wherein:
a) the first polypeptide comprises, from N-terminus to C-terminus: the Fab heavy chain of the first binding domain, the Fc polypeptide chain of the first heavy chain constant region, and the scFv of the third binding domain;
b) the second polypeptide comprises, from N-terminus to C-terminus: the Fab heavy chain of the second binding domain and the Fc polypeptide region of the second heavy chain constant region; and
c) the third polypeptide comprises the first light chain.

48. The multispecific antibody of any one of claims 6-47, wherein the multispecific antibody comprises a first polypeptide, a second polypeptide, and a third polypeptide, wherein:
a) the first polypeptide comprises, from N-terminus to C-terminus: the Fab heavy chain of the first binding domain comprising the amino acid sequence of SEQ ID NO: 53, the Fc polypeptide chain of the first heavy chain constant region comprising the amino acid sequence of SEQ ID NO: 52, and the scFv of the third binding domain comprising the amino acid sequence of SEQ ID NO: 33;
b) the second polypeptide comprises, from N-terminus to C-terminus: the Fab heavy chain of the second binding domain comprising the amino acid sequence of SEQ ID NO: 53 and the Fc polypeptide chain of the second heavy chain constant region comprising the amino acid sequence of SEQ ID NO: 51; and
c) the third polypeptide comprises the first light chain comprising the amino acid sequence of SEQ ID NO: 16.

49. The multispecific antibody of any one of claims 6-47, wherein the multispecific antibody comprises a first polypeptide, a second polypeptide, and a third polypeptide, wherein:
a) the first polypeptide comprises, from N-terminus to C-terminus: the Fab heavy chain of the first binding domain comprising the amino acid sequence of SEQ ID NO: 53, the Fc polypeptide chain of the first heavy chain constant region comprising the amino acid sequence of SEQ ID NO: 56, and the scFv of the third binding domain comprising the amino acid sequence of SEQ ID NO: 33;
b) the second polypeptide comprises, from N-terminus to C-terminus: the Fab heavy chain of the second binding domain comprising the amino acid sequence of SEQ ID NO: 53 and the Fc polypeptide chain of the second heavy chain constant region comprising the amino acid sequence of SEQ ID NO: 55;
c) the third polypeptide comprises the first light chain comprising the amino acid sequence of SEQ ID NO: 16.

50. The multispecific antibody of any one of claims 1-49, wherein the multispecific antibody comprises a first polypeptide, a second polypeptide, and a third polypeptide, wherein:
a) the first polypeptide comprises, from N-terminus to C-terminus: a first heavy chain variable region, a first heavy chain constant region and an scFv comprising a third heavy chain variable region and a third light chain variable region;
b) the second polypeptide comprises, from N-terminus to C-terminus: a second heavy chain variable region and a second heavy chain constant region; and
c) the third polypeptide comprises a first light chain variable region and a first light chain constant region;
wherein the ratio of the first polypeptide to the second polypeptide to the third polypeptide is 1:1:2.

51. The multispecific antibody of any one of claims 47-50, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

52. The multispecific antibody of any one of claims 47-51, comprising at least one post-translational modification of the first polypeptide, second polypeptide, and/or third polypeptide.

53. The multispecific antibody of any one of claims 47-50 and 52, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

54. The multispecific antibody of any one of claims 47-50 and 52, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

55. The multispecific antibody of any one of claims 47-50 and 52, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

56. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

57. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

58. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

59. A multispecific antibody comprising two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

60. The multispecific antibody of any one of claims 1-59, wherein the multispecific antibody is a CD40 agonist in the presence of FAP-expressing cells.

61. The multispecific antibody of any one of claims 1-60, wherein the multispecific antibody activates dendritic cells and macrophages in the presence of FAP-expressing cells.

62. The multispecific antibody of any one of claims 1-61, wherein the multispecific antibody does not compete with CD40L for binding to CD40.

63. The multispecific antibody of any one of claims 1-61, wherein the multispecific antibody binds CD40 with a K_{D} between 20 and 200 nM, or 50 and 200 nM, or 50 and 150 nM, as determined by surface plasmon resonance.

64. The multispecific antibody of any one of claims 1-61, wherein the multispecific antibody binds CD40 expressed on the surface of cells with an EC50 of 1-50 nM, or 1-25 nM, or 3-20 nM, or 3-15 nM.

65. The multispecific antibody of any one of claims 1-64, wherein the multispecific antibody binds FAP with a K_{D} between 0.5 and 10 nM, or 0.5 and 7 nM, or 1 and 5 nM, as determined by surface plasmon resonance.

66. The multispecific antibody of any one of claims 1-64, wherein the multispecific antibody binds FAP expressed on the surface of cells with an EC50 of between 0.5 and 10 nM, or 0.5 and 7 nM, or 1 and 5 nM.

67. A pharmaceutical composition comprising the multispecific antibody of any one of claims 1-66 and a pharmaceutically acceptable carrier.

68. An isolated nucleic acid that encodes the multispecific antibody of any one of claims 1-66.

69. The isolated nucleic acid of claim 68, which is an expression vector.

70. An isolated nucleic acid that encodes the first polypeptide, the second polypeptide, and/or the third polypeptide of the multispecific antibody of any one of claims 47-66.

71. The isolated nucleic acid of claim 70, which is an expression vector.

72. A host cell that expresses the multispecific antibody of any one of claims 1-66.

73. A host cell comprising the nucleic acid of claim 68 or claim 69.

74. A host cell comprising the nucleic acid of claim 70 or claim 71.

75. A host cell comprising a first polynucleotide sequence that encodes the first polypeptide, a second polynucleotide sequence that encodes the second polypeptide, and a third nucleic acid sequence that encodes the third polypeptide, of the multispecific antibody of any one of claims 47-66.

76. A method of producing a multispecific antibody comprising culturing the host cell of any one of claims 72-75 under conditions suitable for expressing the multispecific antibody.

77. The method of claim 76, further comprising isolating the multispecific antibody.

78. An antibody or antigen-binding fragment thereof comprising an antigen binding domain that binds CD40, wherein the antigen binding domain comprises a heavy chain variable region comprising a heavy chain complementarity determining region (HCDR) 1 comprising the amino acid sequence of SEQ ID NO: 1 or 7, a HCDR2 comprising the amino acid sequence of SEQ ID NO: 2 or 8, and a HCDR3 comprising the amino acid sequence of SEQ ID NO: 3 or 9; and a light chain variable region comprising a LCDR1 comprising the amino acid sequence of SEQ ID NO: 4 or 10, a LCDR2 comprising the amino acid sequence of SEQ ID NO: 5 or 11, and a LCDR3 comprising the amino acid sequence of SEQ ID NO: 6 or 12.

79. The antibody or antigen-binding fragment thereof of claim 78, wherein the heavy chain variable region comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and the light chain variable region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

80. The antibody or antigen-binding fragment thereof of claim 78, wherein the heavy chain variable region comprises a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and the light chain variable region comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

81. The antibody or antigen-binding fragment thereof of any one of claims 78-80, wherein the heavy chain variable region comprises an amino acid sequence at least 85%, at least 90%, or at least 95% identical to the amino acid sequence of SEQ ID NO: 13; and the light chain variable region comprises an amino acid sequence at least 85%, at least 90%, or at least 95% identical to the amino acid sequence of SEQ ID NO: 14.

82. The antibody or antigen-binding fragment thereof of any one of claims 78-81, wherein the heavy chain variable region comprises the amino acid sequence of SEQ ID NO: 13, and the light chain variable region comprises the amino acid sequence of SEQ ID NO: 14.

83. The antibody or antigen-binding fragment thereof of any one of claims 78-82, wherein the antibody or antigen-binding fragment thereof is a bispecific antibody.

84. The antibody or antigen-binding fragment thereof of claim 83, wherein the antibody or antigen-binding fragment thereof further comprises a second antigen binding domain that binds FAP.

85. The antibody or antigen-binding fragment thereof of claim 84, wherein the second antigen binding domain comprises a second heavy chain variable region comprising a heavy chain complementarity determining region (HCDR) 1 comprising the amino acid sequence of SEQ ID NO: 17 or 23, a HCDR2 comprising the amino acid sequence of SEQ ID NO: 18 or 24, and a HCDR3 comprising the amino acid sequence of SEQ ID NO: 19 or 25, and a second light chain variable region comprising a LCDR1 comprising the amino acid sequence of SEQ ID NO: 20 or 26, a LCDR2 comprising the amino acid sequence of SEQ ID NO: 21 or 27, and a LCDR3 comprising the amino acid sequence of SEQ ID NO: 22 or 28.

86. The antibody or antigen-binding fragment thereof of claim 84, wherein the second heavy chain variable region comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19; and the second light chain variable region comprises a LCDR1 comprising the amino acid sequence of SEQ ID NO: 20, a LCDR2 comprising the amino acid sequence of SEQ ID NO: 21, and a LCDR3 comprising the amino acid sequence of SEQ ID NO: 22.

87. The antibody or antigen-binding fragment thereof of claim 84, wherein the second heavy chain variable region comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25; and the second light chain variable region comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

88. The antibody or antigen-binding fragment thereof of any one of claims 85-87, wherein the second heavy chain variable region comprises an amino acid sequence at least 85%, at least 90%, or at least 95% identical to the amino acid sequence of SEQ ID NO: 29 or 31; and the second light chain variable region comprises an amino acid sequence at least 85%, at least 90%, or at least 95% identical to the amino acid sequence of SEQ ID NO: 30 or 32.

89. The antibody or antigen-binding fragment thereof of any one of claims 84-88, wherein the second antigen binding domain comprises a second heavy chain variable region comprising the amino acid sequence of SEQ ID NO: 29 or 31; and a second light chain variable region comprising the amino acid sequence of SEQ ID NO: 30 or 32.

90. The antibody or antigen-binding fragment thereof of any one of claims 84-88, wherein the second antigen binding domain comprises a second heavy chain variable region comprising the amino acid sequence of SEQ ID NO: 29; and a second light chain variable region comprising the amino acid sequence of SEQ ID NO: 30.

91. The antibody or antigen-binding fragment thereof of any one of claims 84-88, wherein the second antigen binding domain comprises a second heavy chain variable region comprises the amino acid sequence of SEQ ID NO: 31; and a second light chain variable region comprising the amino acid sequence of SEQ ID NO: 32.

92. The antibody or antigen-binding fragment thereof of any one of claims 84-91, wherein the second antigen binding domain is a Fab, Fab′, F(ab′)₂, Fd, Fv, single-chain Fv (scFv) or disulfide-linked Fv (sdFv).

93. The antibody or antigen-binding fragment thereof of any one of claims 84-92, wherein the second antigen binding domain is a scFv.

94. The antibody or antigen-binding fragment thereof of claim 92 or claim 93, wherein the scFv comprised the amino acid sequence of SEQ ID NO: 33.

95. The antibody or antigen-binding fragment thereof of any one of claims 78-94, wherein the antibody comprises a third antigen binding domain that binds CD40.

96. The antibody or antigen-binding fragment thereof of claim 95, wherein the first antigen binding domain and the third antigen binding domain are the same or different.

97. A pharmaceutical composition comprising the antibody or antigen-binding fragment thereof of any one of claims 78-96 and a pharmaceutically acceptable carrier.

98. An isolated nucleic acid that encodes the antibody or antigen-binding fragment thereof of any one of claims 78-96.

99. The isolated nucleic acid of claim 98, which is an expression vector.

100. A host cell that expresses the antibody of any one of claims 78-96.

101. A host cell comprising the nucleic acid of claim 98 or claim 99.

102. A method of producing an antibody or antigen-binding fragment thereof comprising culturing the host cell of claim 100 or claim 101 under conditions suitable for expressing the antibody.

103. The method of claim 102, further comprising isolating the antibody or antigen-binding fragment thereof.

104. A method of treating cancer comprising administering to a subject in need thereof the multispecific antibody of any one of claims 1-66, the antibody or antigen-binding fragment thereof of any one of claims 78-96, or the pharmaceutical composition of claim 67 or claim 97.

105. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

106. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

107. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

108. A method of treating cancer comprising administering to a subject in need thereof a multispecific antibody, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

109. The method of any one of claims 104-108, wherein the cancer is a solid tumor.

110. The method of any one of claims 104-109, wherein the cancer is gastric cancer or pancreatic cancer.

111. The method of claim 110 wherein the pancreatic cancer is pancreatic ductal adenocarcinoma.

112. The method of any one of claims 104-110, wherein the cancer is selected from non-small cell lung cancer (NSCLC), microsatellite stable (MSS) colorectal carcinoma (CRC), pancreatic ductal adenocarcinoma (PDAC), gastric/gastroesophageal junction adenocarcinoma (G/GEJC), and squamous cell carcinoma of the head and neck (SCCHN).

113. The method of any one of claims 104-112, wherein the cancer is advanced unresectable cancer.

114. The method of any one of claims 104-113, wherein the cancer is metastatic cancer.

115. The method of any one of claims 104-114, wherein the cancer is recurrent cancer.

116. The method of any one of claims 104-115, comprising administering to a subject about 10 mg to about 1000 mg of the multispecific antibody.

117. The method of claim 116, comprising administering to a subject about 10 mg of the multispecific antibody.

118. The method of claim 116, comprising administering to a subject about 30 mg of the multispecific antibody.

119. The method of claim 116, comprising administering to a subject about 90 mg of the multispecific antibody.

120. The method of claim 116, comprising administering to a subject about 250 mg of the multispecific antibody.

121. The method of claim 116, comprising administering to a subject about 500 mg of the multispecific antibody.

122. The method of claim 116, comprising administering to a subject about 1000 mg of the multispecific antibody.

123. The method of any one of claims 104-122, wherein the multispecific antibody is administered in a cycling regimen of one or more 21-day or 28-day cycles, wherein Day 1 is the first day of each cycle.

124. The method of any one of claims 104-123, wherein the multispecific antibody is administered to the subject about once every week, about once every two weeks, about once every three weeks, or about once every four weeks.

125. The method of claim 124, wherein the multispecific antibody is administered to the subject about once every two weeks.

126. The method of claim 124, wherein the multispecific antibody is administered to the subject about once every three weeks.

127. The method of any one of claims 104-126, wherein the multispecific antibody is administered to the subject by intravenous administration.

128. The method of any one of claims 104-126, wherein the multispecific antibody is administered to the subject by subcutaneous administration.

129. The method of any one of claims 104-128, wherein the method further comprises administering at least one PD-1 therapy.

130. The method of claim 129, wherein the PD-1 therapy is an antibody or antigen-binding fragment thereof that binds to PD-1, or an antibody or antigen-binding fragment thereof that binds to PD-L1.

131. The method of claim 129 or claim 130, wherein the multispecific antibody and the PD-1 therapy are administered separately.

132. The method of claim 131, wherein the multispecific antibody and PD-1 therapy are administered sequentially.

133. The method of claim 132, wherein the multispecific antibody is administered after the PD-1 therapy.

134. The method of claim 132, wherein the PD-1 therapy is administered after the multispecific antibody.

135. The method of claim 131, wherein the multispecific antibody and PD-1 therapy are co-administered, wherein the multispecific antibody is in a first bag and the PD-1 therapy is in a second bag, and wherein the multispecific antibody and the PD-1 therapy are administered simultaneously.

136. The method of any one of claims 129-135, wherein the PD-1 therapy is administered to the subject by intravenous administration.

137. The method of any one of claims 129-136, wherein the PD-1 therapy is administered in a cycling regimen of one or more 21-day or 28-day cycles, wherein Day 1 is the first day of each cycle.

138. The method of any one of claims 129-137, wherein the PD-1 therapy is selected from nivolumab, pembrolizumab, cemiplimab, dostarlimab, retifanlimab, toripalimab, atezolizumab, avelumab, and durvalumab.

139. The method of any one of claims 129-138, wherein the PD-1 therapy is nivolumab.

140. The method of claim 138 or claim 139, wherein nivolumab is administered to the subject about once every two weeks at a dose of 240 mg, about once every three weeks at a dose of 360 mg, or about once every four weeks at a dose of 480 mg.

141. The method of claim 138 or claim 139, wherein nivolumab is administered to the subject about once every two weeks at a dose of 240 mg.

142. The method of claim 138 or claim 139, wherein nivolumab is administered to the subject about once every two weeks at a dose of 600 mg.

143. The method of claim 138 or claim 139, wherein nivolumab is administered to the subject about once every two weeks at a dose of 720 mg.

144. The method of claim 138 or claim 139, wherein nivolumab is administered to the subject about once every two weeks at a dose of 960 mg.

145. The method of claim 138 or claim 139, wherein nivolumab is administered to the subject about once every two weeks at a dose of 1200 mg.

146. The method of claim 138 or claim 139, wherein nivolumab is administered to the subject about once every three weeks at a dose of 360 mg.

147. The method of claim 138 or claim 139, wherein nivolumab is administered to the subject about once every three weeks at a dose of 720 mg.

148. The method of claim 138 or claim 139, wherein nivolumab is administered to the subject about once every three weeks at a dose of 900 mg.

149. The method of claim 138 or claim 139, wherein nivolumab is administered to the subject about once every three weeks at a dose of 960 mg.

150. The method of claim 138 or claim 139, wherein nivolumab is administered to the subject about once every three weeks at a dose of 1200 mg.

151. The method of claim 138 or claim 139, wherein nivolumab is administered to the subject about once every four weeks at a dose of 480 mg.

152. The method of claim 138 or claim 139, wherein nivolumab is administered to the subject about once every four weeks at a dose of 720 mg.

153. The method of claim 138 or claim 139, wherein nivolumab is administered to the subject about once every four weeks at a dose of 960 mg.

154. The method of claim 138 or claim 139, wherein nivolumab is administered to the subject about once every four weeks at a dose of 1200 mg.

155. The method of any one of claims 138-154, wherein nivolumab is administered intravenously.

156. The method of any one of claims 138-154, wherein nivolumab is administered subcutaneously.

157. The method of any one of claims 138-154 and 156, wherein nivolumab is co-formulated with hyaluronidase.

158. The method of claim 157, wherein hyaluronidase is at a dose of about 10,000 units to about 20,000 units, inclusive.

159. The method of claim 157 or claim 158, wherein hyaluronidase is at a dose of about 10,000 units; about 12,000 units; 15,000 units; about 16,000 units; or about 20,000 units.

160. The method of any one of claims 157-159, wherein nivolumab co-formulated with hyaluronidase is administered to the subject about once every two weeks at a dose of 600 mg nivolumab and a dose of about 10,000 units hyaluronidase.

161. The method of any one of claims 157-159, wherein nivolumab co-formulated with hyaluronidase is administered to the subject about once every three weeks at a dose of 900 mg nivolumab and a dose of about 15,000 units hyaluronidase.

162. The method of any one of claims 157-159, wherein nivolumab co-formulated with hyaluronidase is administered to the subject about once every four weeks at a dose of 1200 mg nivolumab and about 20,000 units hyaluronidase.

163. The method of any one of claims 157-159, wherein nivolumab co-formulated with hyaluronidase is administered to the subject about once every two to four weeks at a dose of 720 mg nivolumab and about 20,000 units hyaluronidase.

164. The method of any one of claims 157-159, wherein nivolumab co-formulated with hyaluronidase is administered to the subject about once every two to four weeks at a dose of 960 mg nivolumab and about 20,000 units hyaluronidase.

165. The method of any one of claims 157-159, wherein nivolumab co-formulated with hyaluronidase is administered to the subject about once every two to four weeks at a dose of 1200 mg nivolumab and about 20,000 units hyaluronidase.

166. The method of any one of claims 157-159, wherein nivolumab co-formulated with hyaluronidase is administered to the subject about once every two to four weeks at a dose of 720 mg nivolumab and about 12,000 units hyaluronidase.

167. The method of any one of claims 157-159, wherein nivolumab co-formulated with hyaluronidase is administered to the subject about once every two to four weeks at a dose of 960 mg nivolumab and about 16,000 units hyaluronidase.

168. The method of any one of claims 157-159, wherein the nivolumab co-formulated with hyaluronidase is administered to the subject about once every two to four weeks at a dose of 1200 mg nivolumab and about 20,000 units hyaluronidase.

169. The method of any one of claims 160-168, wherein the once every two to four weeks is once every two weeks.

170. The method of any one of claims 160-168, wherein the once every two to four weeks is once every three weeks.

171. The method of any one of claims 160-168, wherein the once every two to four weeks is once every four weeks.

172. The method of any one of claims 129-138, wherein the PD-1 therapy is pembrolizumab, and wherein pembrolizumab is administered to the subject about once every three weeks at a dose of 200 mg or about once every six weeks at a dose of 400 mg.

173. The method of any one of claims 129-138, wherein the PD-1 therapy is cemiplimab, and wherein cemiplimab is administered to the subject about once every three weeks at a dose of 350 mg.

174. The method of any one of claims 129-138, wherein the PD-1 therapy is dostarlimab, and wherein dostarlimab is administered to the subject about once every three weeks at a dose of 500 mg for dose 1 through dose 4 and about once every six weeks at a dose of 1000 mg for dose 5 onwards.

175. The method of any one of claims 129-138, wherein the PD-1 therapy is retifanlimab, and wherein retifanlimab is administered to the subject about once every four weeks at a dose of 500 mg.

176. The method of any one of claims 129-138, wherein the PD-1 therapy is toripalimab, and wherein toripalimab is administered to the subject about once every two weeks at a dose of 3 mg/kg.

177. The method of any one of claims 129-138, wherein the PD-1 therapy is atezolizumab, and wherein atezolizumab is administered to the subject about once every two weeks at a dose of 840 mg, once every three weeks at a dose of 1200 mg, or once every four weeks at a dose of 1680 mg.

178. The method of any one of claims 129-138, wherein the PD-1 therapy is avelumab, and wherein avelumab is administered to the subject about once every two weeks at a dose of 800 mg.

179. The method of any one of claims 129-138, wherein the PD-1 therapy is durvalumab, and wherein durvalumab is administered to the subject about once every two weeks at a dose of 10 mg/kg.

180. The method of any one of claims 104-179, wherein the method further comprises administering a chemotherapy.

181. The method of claim 180, wherein the chemotherapy comprises capecitabine and oxaliplatin administered in a cycling regimen of one or more 21-day cycles, wherein Day 1 is the first day of each cycle.

182. The method of claim 180 or claim 181, wherein capecitabine is administered to the subject about twice daily (BID) on Days 1 to 14 of each 21-day cycle at a dose of about 500 to about 1000 mg/m² and oxaliplatin is administered to the subject on Day 1 of each 21-day cycle at a dose of about 100 to about 150 mg/m².

183. The method of any one of claims 180-182, wherein capecitabine is administered to the subject about twice daily (BID) on Days 1 to 14 of each 21-day cycle at a dose of about 500 mg/m², about 750 mg/m², about 850 mg/m², or about 1000 mg/m² and oxaliplatin is administered to the subject on Day 1 of each 21-day cycle at a dose of about 130 mg/m².

184. The method of any one of claims 180-182, wherein capecitabine is administered to the subject about twice daily (BID) on Days 1 to 14 of each 21-day cycle at a dose of about 850 to about 1000 mg/m² and oxaliplatin is administered to the subject on Day 1 of each 21-day cycle at a dose of about 130 mg/m².

185. The method of any one of claims 180-184, wherein capecitabine is administered to the subject about twice daily (BID) on Days 1 to 14 of each 21-day cycle at a dose of about 1000 mg/m² and oxaliplatin is administered to the subject on Day 1 of each 21-day cycle at a dose of about 130 mg/m².

186. The method of claim 180, wherein the chemotherapy comprises oxaliplatin, folinic acid or a therapeutic equivalent thereof, and fluorouracil administered in a cycling regimen of one of more 28-day cycles, wherein Day 1 is the first day of each cycle.

187. The method of claim 186, wherein oxaliplatin is administered to the subject on Days 1 and 15 of each 28-day cycle at a dose of about 50 mg/m² to about 90 mg/m²; folinic acid or a therapeutic equivalent thereof is administered to the subject on Days 1 and 15 of each 28-day cycle at a dose of about 200 mg/m² to about 450 mg/m²; and fluorouracil is administered to the subject as a bolus on Days 1 and 15 of each 28-day cycle at a dose of about 200 mg/m² to about 450 mg/m² and as a continuous infusion starting on Days 1 and 15 of each 28-day cycle at a dose of about of about 1600 mg/m²/48 hours to about 2500 mg/m²/48 hours.

188. The method of claim 186 or claim 187, wherein oxaliplatin is administered to the subject on Days 1 and 15 of each 28-day cycle at a dose of about 50 mg/m², about 70 mg/m², or about 85 mg/m²; folinic acid or a therapeutic equivalent thereof is administered to the subject on Days 1 and 15 of each 28-day cycle at a dose of about 200 mg/m², about 300 mg/m², or about 400 mg/m²; and fluorouracil is administered to the subject as a bolus on Days 1 and 15 of each 28-day cycle at a dose of about 200 mg/m², about 300 mg/m², or about 400 mg/m² and as a continuous infusion starting on Days 1 and 15 of each 28-day cycle at a dose of about 1600 mg/m²/48 hours, about 2000 mg/m²/48 hours or about 2400 mg/m²/48 hours.

189. The method of any one of claims 186-188, wherein oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours.

190. The method of any one of claims 104-140, wherein the multispecific antibody is administered about once every two weeks at a dose of 30 mg in combination with nivolumab, wherein nivolumab is administered to the subject about once every two weeks at a dose of 240 mg.

191. The method of any one of claims 104-140, wherein the multispecific antibody is administered about once every two weeks at a dose of 30 mg in combination with nivolumab, wherein nivolumab is administered to the subject about once every three weeks at a dose of 360 mg.

192. The method of any one of claims 104-140, wherein the multispecific antibody is administered about once every two weeks at a dose of 30 mg in combination with nivolumab, wherein nivolumab is administered to the subject about once every four weeks at a dose of 480 mg.

193. The method of any one of claims 104-140, wherein the multispecific antibody is administered about once every two weeks at a dose of 90 mg in combination with nivolumab, wherein nivolumab is administered to the subject about once every two weeks at a dose of 240 mg.

194. The method of any one of claims 104-140, wherein the multispecific antibody is administered about once every two weeks at a dose of 90 mg in combination with nivolumab, wherein nivolumab is administered to the subject about once every three weeks at a dose of 360 mg.

195. The method of any one of claims 104-140, wherein the multispecific antibody is administered about once every two weeks at a dose of 90 mg in combination with nivolumab, wherein nivolumab is administered to the subject about once every four weeks at a dose of 480 mg.

196. The method of any one of claims 104-140, wherein the multispecific antibody is administered about once every two weeks at a dose of 250 mg in combination with nivolumab, wherein nivolumab is administered to the subject about once every two weeks at a dose of 240 mg.

197. The method of any one of claims 104-140, wherein the multispecific antibody is administered about once every two weeks at a dose of 250 mg in combination with nivolumab, wherein nivolumab is administered to the subject about once every three weeks at a dose of 360 mg.

198. The method of any one of claims 104-140, wherein the multispecific antibody is administered about once every two weeks at a dose of 250 mg in combination with nivolumab, wherein nivolumab is administered to the subject about once every four weeks at a dose of 480 mg.

199. The method of any one of claims 104-140, wherein the multispecific antibody is administered about once every two weeks at a dose of 500 mg in combination with nivolumab, wherein nivolumab is administered to the subject about once every two weeks at a dose of 240 mg.

200. The method of any one of claims 104-140, wherein the multispecific antibody is administered about once every two weeks at a dose of 500 mg in combination with nivolumab, wherein nivolumab is administered to the subject about once every three weeks at a dose of 360 mg.

201. The method of any one of claims 104-140, wherein the multispecific antibody is administered about once every two weeks at a dose of 500 mg in combination with nivolumab, wherein nivolumab is administered to the subject about once every four weeks at a dose of 480 mg.

202. The method of any one of claims 104-140, wherein the multispecific antibody is administered about once every two weeks at a dose of 1000 mg in combination with nivolumab, wherein nivolumab is administered to the subject about once every two weeks at a dose of 240 mg.

203. The method of any one of claims 104-140, wherein the multispecific antibody is administered about once every two weeks at a dose of 1000 mg in combination with nivolumab, wherein nivolumab is administered to the subject about once every three weeks at a dose of 360 mg.

204. The method of any one of claims 104-140, wherein the multispecific antibody is administered about once every two weeks at a dose of 1000 mg in combination with nivolumab, wherein nivolumab is administered to the subject about once every four weeks at a dose of 480 mg.

205. The method of any one of claims 104-140, wherein the multispecific antibody is administered in combination with nivolumab and a chemotherapy comprising capecitabine and oxaliplatin in a cycling regimen of one or more 21-day cycles, wherein Day 1 is the first day of each cycle, wherein the multispecific antibody is administered to the subject on Day 1 of each 21-day cycle at a dose of about 10 mg to about 1000 mg; wherein nivolumab is administered to the subject on Day 1 of each 21-day cycle at a dose of about 360 mg; wherein capecitabine is administered to the subject about twice daily (BID) on Days 1 to 14 of each 21-day cycle at a dose of about 1000 mg/m²; and oxaliplatin is administered to the subject on Day 1 of each 21-day cycle at a dose of about 130 mg/m².

206. The method of any one of claims 104-140, wherein the multispecific antibody is administered in combination with nivolumab and a chemotherapy comprising oxaliplatin, folinic acid or a therapeutic equivalent thereof, and fluorouracil in a cycling regimen of one or more 28-day cycles, wherein Day 1 is the first day of each cycle, wherein the multispecific antibody is administered to the subject on Days 1 and 15 of each 28-day cycle at a dose of about 10 mg to about 1000 mg; wherein nivolumab is administered to the subject on Days 1 and 15 of each 28-day cycle at a dose of about 240 mg; wherein oxaliplatin is administered to the subject on Days 1 and 15 of each 28-day cycle at a dose of about 85 mg/m²; folinic acid or a therapeutic equivalent thereof is administered to the subject on Days 1 and 15 of each 28-day cycle at a dose of about 400 mg/m²; and fluorouracil is administered to the subject as a bolus on Days 1 and 15 of each 28-day cycle at a dose of about 400 mg/m² and as a continuous infusion starting on Days 1 and 15 of each 28-day cycle at a dose of about 2400 mg/m²/48 hours.

207. The method of claim 205 or claim 206, wherein the multispecific antibody is administered to the subject at a dose of about 10 mg.

208. The method of claim 205 or claim 206, wherein the multispecific antibody is administered to the subject at a dose of about 30 mg.

209. The method of claim 205 or claim 206, wherein the multispecific antibody is administered to the subject at a dose of about 90 mg.

210. The method of claim 205 or claim 206, wherein the multispecific antibody is administered to the subject at a dose of about 250 mg.

211. The method of claim 205 or claim 206, wherein the multispecific antibody is administered to the subject at a dose of about 500 mg.

212. The method of claim 205 or claim 206, wherein the multispecific antibody is administered to the subject at a dose of about 1000 mg.

213. The method of any one of claims 123-212, wherein each cycle of the cycling regimen is the same.

214. The method of any one of claims 104-213, wherein the cancer is NSCLC and the subject has not yet received treatment.

215. The method of any one of claims 104-213, wherein the cancer is NSCLC and the subject:
a) previously received platinum doublet-based chemotherapy and then progressed; or
b) was intolerant to platinum doublet-based chemotherapy.

216. The method of any one of claims 104-213 and 215, wherein the cancer is NSCLC and the subject:
a) previously received at least two prior lines of systemic therapy for advanced or metastatic disease and then progressed; or
b) was intolerant to at least two prior lines of systemic therapy for advanced or metastatic disease.

217. The method of any one of claims 104-213, 215, and 216, wherein the cancer is NSCLC and the subject has recurrent or progressive disease after completing platinum-based chemotherapy for local disease.

218. The method of any one of claims 104-213 and 215-217, wherein the cancer is NSCLC and the subject:
a) is administered the multispecific antibody in combination with nivolumab, and
b) has received previous treatment with a PD-1 therapy.

219. The method of any one of claims 104-213 and 215-218, wherein the cancer is NSCLC and the subject:
a) has one or more mutations in a protein selected from EGFR, ALK, ROS1, and RET, and
b) has received and progressed on, has been intolerant to, or was not a candidate for therapy with a tyrosine kinase inhibitor.

220. The method of any one of claims 104-213, wherein the cancer is SCCHN and the SCCHN is of the oral cavity, pharynx, or larynx.

221. The method of any one of claims 104-213 and 220, wherein the cancer is SCCHN of the oral cavity, pharynx, or larynx and the subject has not yet received treatment.

222. The method of any one of claims 104-213 and 220, wherein the cancer is SCCHN of the oral cavity, pharynx, or larynx and the subject:
a) previously received a platinum-containing regimen and then progressed, or b) was intolerant to a platinum-containing regimen.

223. The method of any one of claims 104-213, 220, and 222, wherein the cancer is SCCHN of the oral cavity, pharynx, or larynx and the subject:
a) is administered the multispecific antibody in combination with nivolumab, and b) has received previous treatment with a PD-1 therapy.

224. The method of any one of claims 104-213, wherein the cancer is PDAC and the subject has not yet received treatment.

225. The method of any one of claims 104-213, wherein the cancer is PDAC and the subject:
a) previously received at least one prior chemotherapy and then progressed; or
b) was intolerant to at least one prior chemotherapy.

226. The method of any one of claims 104-213, wherein the cancer is G/GEJC and the subject has not yet received treatment.

227. The method of any one of claims 104-213, wherein the cancer is G/GEJC and the subject:
a) previously received at least one prior standard treatment regimen in the advanced or metastatic setting and then progressed;
b) was intolerant to at least one prior standard treatment regimen in the advanced or metastatic setting; or
c) has progressed within 6 months of adjuvant therapy.

228. The method of any one of claims 104-213 and 227, wherein the cancer is G/GEJC and the subject:
a) is administered the multispecific antibody in combination with nivolumab, and b) has received previous treatment with a PD-1 therapy.

229. The method of any one of claims 104-213, 227, and 228, wherein the G/GEJC is human epidermal growth factor receptor 2 (HER2)-positive G/GEJC, and wherein the subject has received prior treatment with a HER2 inhibitor.

230. The method of claim 229, wherein the HER2 inhibitor is trastuzumab.

231. The method of any one of claims 104-213, wherein the cancer is MSS CRC and the subject has not yet received treatment.

232. The method of any one of claims 104-213, wherein the cancer is MSS CRC and the subject:
a) previously received at least one standard systemic therapy for metastatic and/or unresectable disease and then progressed;
b) was intolerant to one standard systemic therapy for metastatic and/or unresectable disease; or
c) has progressed within 6 months of adjuvant therapy.

233. The method of any one of claims 104-213 and 232, wherein the cancer is MSS CRC and the subject has received prior treatment with fluoropyrimidine, oxaliplatin, and irinotecan given as a single regimen or over multiple regimens.

234. The method of any one of claims 104-213, 232, and 233, wherein the cancer is MSS CRC and the subject has proficient mismatch repair (MMR).

235. The method of any one of claims 104-213 and 232-234, wherein the cancer is MSS CRC and the subject has wild-type RAS and was previously treated with an anti-EGFR therapy.

236. The method of claim 235, wherein the anti-EGFR therapy is cetuximab or panitumumab.

237. The method of any one of claims 104-236, wherein treatment of the subject in need thereof is continued for about 1 month to about 24 months.

238. The method of any one of claims 104-237, wherein treatment of the subject in need thereof is continued for at least 1, 2, 3, 4, 5, or 6 months.

239. The method of any one of claims 104-238, wherein treatment of the subject in need thereof is continued until the subject achieves a complete response.

240. The method of claim 127, wherein the intravenous administration is completed over 30 minutes.

241. A multispecific antibody for use in treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

242. A multispecific antibody for use in treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

243. A multispecific antibody for use in treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

244. A multispecific antibody for use in treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.

245. Use of a multispecific antibody in the manufacture of a medicament for treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 15, and the third polypeptide comprises the amino acid sequence of SEQ ID NO: 16.

246. Use of a multispecific antibody in the manufacture of a medicament for treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 34, the second polypeptide comprises the amino acid sequence of SEQ ID NO: 43, and the third polypeptide comprises the amino acid sequence of SEQ ID NO: 16.

247. Use of a multispecific antibody in the manufacture of a medicament for treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 15, and the third polypeptide comprises the amino acid sequence of SEQ ID NO: 16.

248. Use of a multispecific antibody in the manufacture of a medicament for treating a subject according to the method of any one of claims 104-240, wherein the multispecific antibody comprises two antigen binding domains that bind CD40 and one antigen binding domain that binds FAP, wherein the multispecific antibody comprises a first polypeptide, a second polypeptide, and a third polypeptide at a ratio of 1:1:2, wherein the first polypeptide comprises the amino acid sequence of SEQ ID NO: 58, the second polypeptide comprises the amino acid sequence of SEQ ID NO: 43, and the third polypeptide comprises the amino acid sequence of SEQ ID NO: 16.
